(19) United States
(12) Patent Application Publication
(10) Pub. No.: US 2026/0061093 A1
(43) Pub. Date: Mar. 5, 2026

(54) LIPID ANALOGS, LIPOSOMES COMPRISING SAME AND USES THEREOF

(71) Applicant: LipoSphere Ltd., Givat Shmuel (IL)

(72) Inventors: Assaf J. HARNOY, Herzeliya (IL); Ronit GOLDBERG, Givat Shmuel (IL); Sabrina JAHN, Hindhead (GB); Asaf GRUPI, Ness Ziona (IL)

(73) Assignee: LipoSphere Ltd., Givat Shmuel (IL)

(21) Appl. No.: 19/107,083

(22) PCT Filed: Aug. 30, 2023
(86) PCT No.: PCT/IL2023/050927

§ 371 Date: Feb. 27, 2025

Related U.S. Application Data
(60) Provisional application No. 63/402,097, filed on Aug. 30, 2022.
(60) Provisional application No. 63/427,943, filed on Nov. 25, 2022.

(51) Int. Cl. A61L 27/16; A61L 27/54; C08L 43/02

(52) U.S. Cl. CPC A61L 27/16; A61L 27/54; C08L 43/02; A61L 2300/22; A61L 2400/06; A61L 2400/10; A61L 2430/24

(57) ABSTRACT

Newly designed polymeric compounds (lipid-polymer conjugates), which are usable for forming lipid bilayers and liposomes comprising same, lipid bilayers and liposomes formed thereby and uses thereof are disclosed. Newly designed processes of preparing polymeric compounds (lipid-polymer conjugates) are also disclosed.

### RELATED APPLICATION/S

This application claims the benefit of priority of U.S. Patent Application No. 63/402,097 filed on Aug. 30, 2022, and of U.S. Patent Application No. 63/427,943 filed on Nov. 25, 2022, the contents of which are incorporated herein by reference in their entirety.

### FIELD AND BACKGROUND OF THE INVENTION

The present invention, in some embodiments thereof, relates to material science and, more particularly, but not exclusively, to novel polymeric compounds usable, inter alia, for forming liposomes, and to uses of such liposomes in, for example, biomedical applications.

Phosphatidylcholine (PC) liposomes at surfaces are known to be extremely good lubricants, even at high pressures [Goldberg et al., Adv Materials 2011, 23:3517-3521; Goldberg et al., Biophys J 2011, 100:2403-2411; Sorkin et al., Biomaterials 2014, 34:5465-5475].

Aggregation of liposomes into macroscopic aggregates can interfere with the use of liposomes in different ways. Large aggregates can precipitate and sediment out of a dispersion, rendering the dispersion unusable; aggregates larger than about 200-300 nm scatter visible light, leading to turbidity, which may interfere with a use of liposomes in which transparency is important; and furthermore, large aggregates injected into the body are more prone to protein adsorption, and to attack and removal by macrophages [Moghimi & Szebeni, Prog Lipid Res 2003, 42:463-478].

PEGylated PC small unilamellar vesicles (SUVs) have been used for drug delivery, wherein PEG brushes are incorporated in the membrane bilayer; these brushes extend out from the SUV surfaces and sterically-stabilize them against aggregation [Harris & Chess, Nat Rev Drug Discov 2003, 2:214-221]. However, PEGylation was reported to reduce the efficiency of SUVs for lubrication purposes at high pressures (such as in joints), as the PEG chains are not highly hydrated and do not in themselves form good lubricants at high pressures [Goldberg et al., Adv Materials 2011, 23:3517-3521].

U.S. Pat. No. 8,617,592 describes block copolymers and conjugates comprising a zwitterionic poly(carboxybetaine), poly(sulfobetaine) or poly(phosphobetaine) block, and a hydrophobic block, which self-assemble into particles, and the use of such particles for delivering therapeutic and diagnostic agents.

Chen et al. [Science 2009, 323:1698-1702] describes effective lubrication by poly[2-(methacryloyloxy)ethyl phosphorylcholine] (PMPC) brushes, and attributes this phenomenon to strong hydration of the zwitterionic monomers.

WO 2017/109784 describes the design and preparation of polymeric compounds which bear phosphocholine analogs as pendant groups and are conjugated to a lipid moiety. It further describes liposomes comprising such compounds which exhibit enhanced stability in an aqueous environment.

WO 2018/150429 describes the use of lipid-derived polymeric compounds as described in WO 2017/109784 in delivering therapeutically active agents to a subject's bodily site, and thereby in the treatment of medical conditions treatable by the therapeutically active agent.

Additional background art includes Goldberg & Klein [Chem Phys Lipids 2012, 165:374-381]; WO 2011/158237, WO 2015/001564, WO 2015/193887, WO 2015/193888, WO 2016/051413 and WO 2018/150429.

### SUMMARY OF THE INVENTION

According to an aspect of some embodiments of the present invention there is provided a polymeric compound represented by Formula I:

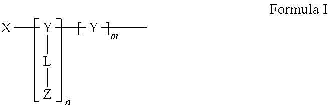

wherein:
m is zero or a positive integer;
n is an integer which is at least 2, at least 5, preferably at least 10 (e.g., of from 10 to 200);
Y is a backbone unit which forms a polymeric backbone of the polymeric compound;
L is absent or is a linking moiety; and
Z has the general Formula II:

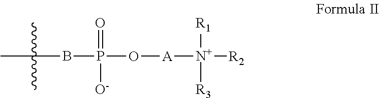

wherein:
the dashed (curved) line denotes an attachment point to the respective Y backbone unit or to the linking moiety L, if present;
A is a substituted or unsubstituted hydrocarbon;
B is an oxygen atom or is absent;
R₁-R₃ are each independently selected from the group consisting of hydrogen, alkyl, cycloalkyl, heteroalicyclic, aryl and heteroaryl;
X is a lipid moiety represented by Formula IV:

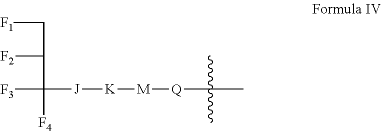

wherein:
the dashed (curved) line denotes an attachment point to the polymeric backbone;
F₁, F₂, F₃ and F₄ are each independently selected from the group consisting of hydrogen, alkyl, alkenyl, alkynyl, acyl, alkoxy, thioalkoxy, carboxy, thiocarboxy, wherein at least one of F₁, F₂, F₃ and F₄ is not hydrogen and is of at least 10 carbon atoms in length;
J is —O—P(═O)(OH)—O— or absent;
K is a substituted or unsubstituted hydrocarbon from 1 to 10 carbon atoms in length or absent;
M is a linking group selected from the group consisting of —O—, —S—, amino, sulfinyl, sulfonyl, phosphate, phosphonyl, phosphinyl, carbonyl, thiocarbonyl, urea, thiourea, carbamyl, thiocarbamyl, amido, carboxy, and sulfonamide, or absent; and
Q is a substituted or unsubstituted hydrocarbon from 1 to 10 carbon atoms in length, or absent,
wherein when M is absent, Q is also absent, and when J is absent, M is not absent,
provided that:
when J is —O—P(═O)(OH)—O—, M is other than amido and/or Q comprises an aryl moiety.

According to some of any of the embodiments described herein, at least one of F₁, F₂, F₃ and F₄ is an alkoxy, thioalkoxy, acyl or carboxy of at least 10 carbon atoms in length.

According to some of any of the embodiments described herein, at least one of F₁, F₂, F₃ and F₄ is derived from a fatty acid selected from the group consisting of lauroyl, myristoyl, palmitoyl, stearoyl, palmitoleoyl, oleoyl, and linoleoyl.

According to some of any of the embodiments described herein, M is carboxy.

According to some of any of the embodiments described herein, K is an alkyl.

According to some of any of the embodiments described herein, J is —P(═O)(OH)—O—; M is amido; and Q is a hydrocarbon substituted by at least one aryl (e.g., phenyl).

According to some of any of the embodiments described herein, Q is a methylene substituted by at least one aryl.

According to some of any of the embodiments described herein, J is absent.

According to some of any of the embodiments described herein, J and K are each absent.

According to some of any of the embodiments described herein, J and K are each absent and M is carboxy.

According to some of any of the embodiments described herein, at least one, or at least two, of F₁, F₂, F₃ and F₄ is independently the thioalkoxy.

According to some of any of the embodiments described herein, at least one, or at least two, of F₁, F₂, F₃ and F₄ is independently the carboxy.

According to some of any of the embodiments described herein, at least one or both of F₁ and F₂ is the carboxy and at least one of F₃ and F₄ is an alkyl.

According to some of any of the embodiments described herein, Q is —C(CH₃)₂—.

According to some of any of the embodiments described herein, Y is a substituted or unsubstituted alkylene unit.

According to some of any of the embodiments described herein, Y is a substituted or unsubstituted ethylene unit.

According to some of any of the embodiments described herein, Y has the formula —CR₄R₅-CR₆D-, wherein:
when Y is a backbone unit which is not attached to the L or the Z, D is R₇; and when Y is a backbone unit which is attached to the L or the Z, D is a covalent bond or a linking group attaching Y to the L or the Z, the linking group being selected from the group consisting of —O—, —S—, alkylene, arylene, sulfinyl, sulfonyl, phosphate, phosphonyl, phosphinyl, carbonyl, thiocarbonyl, urea, thiourea, O-carbamyl, N-carbamyl, O-thiocarbamyl, N-thiocarbamyl, C-amido, N-amido, C-carboxy, O-carboxy, sulfonamido, and amino; and
R₄-R₇ are each independently selected from the group consisting of hydrogen, alkyl, alkenyl, alkynyl, cycloalkyl, aryl, heteroaryl, heteroalicyclic, halo, hydroxy, alkoxy, aryloxy, thiohydroxy, thioalkoxy, thioaryloxy, sulfinyl, sulfonyl, cyano, nitro, azide, azo, phosphate, phosphonyl, phosphinyl, oxo, carbonyl, thiocarbonyl, urea, thiourea, O-carbamyl, N-carbamyl, O-thiocarbamyl, N-thiocarbamyl, C-amido, N-amido, C-carboxy, O-carboxy, sulfonamido, and amino.

According to some of any of the embodiments described herein, R₄-R₇ are each independently selected from hydrogen and alkyl.

According to some of any of the embodiments described herein, R₄ and R₅ are each hydrogen.

According to some of any of the embodiments described herein, R₆ is hydrogen.

According to some of any of the embodiments described herein, the linking group is selected from the group consisting of —O—, —C(═O)O—, C(═O)NH— and phenylene.

According to some of any of the embodiments described herein, the linking group is —C(═O)O—.

According to some of any of the embodiments described herein, L is a substituted or unsubstituted hydrocarbon from 1 to 10 carbon atoms in length.

According to some of any of the embodiments described herein, L is a substituted or unsubstituted ethylene group.

According to some of any of the embodiments described herein, B is an oxygen atom.

According to some of any of the embodiments described herein, A is a substituted or unsubstituted hydrocarbon from 1 to 4 carbon atoms in length.

According to some of any of the embodiments described herein, A is a substituted or unsubstituted ethylene group.

According to some of any of the embodiments described herein, R₁-R₃ are each independently hydrogen or C₁₋₄-alkyl.

According to some of any of the embodiments described herein, R₁-R₃ are each methyl.

According to some of any of the embodiments described herein, n ranges from 10 to 200.

According to some of any of the embodiments described herein, n is at least 30.

According to some of any of the embodiments described herein, n ranges from 30 to 70.

According to some of any of the embodiments described herein, n is at least 50, or at least 60.

According to some of any of the embodiments described herein, n ranges from 50 to 150, or from 50 to 80.

According to some of any of the embodiments described herein, n is at least 80.

According to some of any of the embodiments described herein, n ranges from 80 to 120.

According to some of any of the embodiments described herein, n ranges from 10 to 50.

According to some of any of the embodiments described herein, m ranges from 0 to 50.

According to some of any of the embodiments described herein, at least a portion of the backbone units Y, the L and/or the Z comprise at least one targeting moiety, as described herein.

According to an aspect of some embodiments of the present invention there is provided a lipid bilayer comprising at least one bilayer-forming lipid and the polymeric compound as described herein in any of the respective embodiments and any combination thereof.

According to some of any of the embodiments described herein, a mol ratio of the at least one bilayer-forming lipid and the polymeric compound is in a range of from 5:1 to 5,000:1, or from 10:1 to 1,000:1, or 10:1 to 100:1 or from 10:1 to 50:1 (e.g., 30:1 to 40:1), or from 100:1 to 200:1.

According to some of any of the embodiments described herein, the at least one bilayer-forming lipid comprises at least one zwitterionic glycerophospholipid.

According to some of any of the embodiments described herein, the at least one bilayer-forming lipid further comprises a negatively charged bilayer-forming lipid (e.g., DPPG).

According to some of any of the embodiments described herein, an amount of the negatively charged bilayer-forming lipid ranges from 0.1 to 40, or from 1 to 40, or from 1 to 20, mol % of the lipid bilayer.

According to some of any of the embodiments described herein, n is least 50.

According to an aspect of some embodiments of the present invention there is provided a liposome comprising at least one lipid bilayer as described herein in any of the respective embodiments and any combination thereof.

According to some of any of the embodiments described herein, the liposome further comprises at least one functional moiety or agent, bound to a surface of the liposome and/or within a lipid bilayer and/or core of the liposome.

According to some of any of the embodiments described herein, the functional moiety or agent is a therapeutically active agent or moiety thereof, a labeling moiety or agent and/or a targeting moiety or agent.

According to an aspect of some embodiments of the present invention there is provided a composition comprising liposomes as described herein in any of the respective embodiments and any combination thereof and a carrier, preferably an aqueous carrier.

According to some of any of the embodiments described herein, the composition is a sterile composition.

According to some of any of the embodiments described herein, the composition is a lubricant composition.

According to some of any of the embodiments described herein, the lubricant composition further comprises a water-soluble polymer.

According to some of any of the embodiments described herein, the lubricant composition is for lubricating a physiological surface, wherein the carrier is a physiologically acceptable carrier.

According to an aspect of some embodiments of the present invention there is provided a method of reducing a friction coefficient of a surface, the method comprising contacting the surface with liposomes as described herein in any of the respective embodiments and any combination thereof.

According to some of any of the embodiments described herein, the method comprises contacting the surface with a composition comprising the liposomes and a carrier, preferably an aqueous carrier.

According to some of any of the embodiments described herein, the method further comprises contacting the surface with a water-soluble polymer.

According to some of any of the embodiments described herein, the surface is a hydrogel surface.

According to some of any of the embodiments described herein, the surface is a contact lens surface.

According to some of any of the embodiments described herein, the surface is a physiological surface, and the carrier is a physiologically acceptable carrier.

According to some of any of the embodiments described herein, the surface is an articular surface of a synovial joint.

According to an aspect of some embodiments of the present invention there is provided a liposome as described herein in any of the respective embodiments and any combination thereof, for use in the treatment of a synovial joint disorder associated with an increased friction coefficient of an articular surface in the synovial joint.

According to an aspect of some embodiments of the present invention there is provided a method of inhibiting biofilm formation on a surface of a substrate, the method comprising contacting the substrate with a composition which comprises liposomes as described herein in any of the respective embodiments and any combination thereof.

According to an aspect of some embodiments of the present invention there is provided an article of manufacture comprising a composition-of-matter, the composition-of-matter comprising a substrate coated, on at least a portion of a surface thereof, by a lipid bilayer or a liposome, as described herein in any of the respective embodiments and any combination thereof.

According to an aspect of some embodiments of the present invention there is provided a lipid bilayer, a liposome or a composition comprising same, as described herein in any of the respective embodiments and any combination thereof, for use in treating a synovial joint disorder.

According to some of any of the embodiments described herein, the treating comprises intra-articular administration of the lipid bilayer, the liposome or the composition.

According to an aspect of some embodiments of the present invention there is provided a liposome or a composition comprising same, as described herein in any of the respective embodiments and any combination thereof, wherein the liposome has a therapeutically active agent associated therewith, the liposome or the composition being for use in delivering the therapeutically active agent to a bodily site of a subject.

According to some of any of the embodiments described herein, the liposome or the composition is for use in the treatment of a medical condition treatable by the therapeutically active agent in the subject.

According to an aspect of some embodiments of the present invention there is provided a process of preparing the polymeric compound as described herein in any of the respective embodiments and any combination thereof, the process comprising contacting an initiator compound having Formula V:

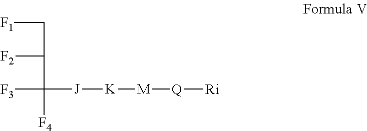

wherein:
F₁, F₂, F₃, F₄, J, K, M and Q are as defined for Formula IV; and
Ri is an electron transfer functional group,
with a plurality of monomers that form the —[Y-L-Z]n-[Y]m- polymeric backbone,
under conditions that promote atom transfer radical polymerization (ATRP).

According to some of any of the embodiments described herein, the ATRP is ARGET-ATRP.

According to some of any of the embodiments described herein, the process further comprises isolating the polymeric compound.

According to some of any of the embodiments described herein, the isolating is by precipitation.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 (Background Art) is a scheme presenting the preparation of a lipid-containing polymer compound (LPC) DSPE-pMPC from a phospholipid distearoylphosphatidylethanolamine (DSPE) and a phosphocholine derivatized-MPC (O-(2-methacryloyloxyethyl)phosphorylcholine) via a brominated derivatized-DSPE (DSPE-Br), as described in WO 2017/109784.

FIG. 2 is a scheme presenting an exemplary two-step synthesis of the LPC DPPE-dm-pMPC: synthesis of a DPPE-dm-Br initiator and polymerization of MPC using the DPPE-dm-Br initiator via atom-transfer radical polymerization (ATRP) to provide the LPC DPPE-dm-pMPC.

FIGS. 3A-B present a general scheme presenting the catalytic process of activators regenerated by electron transfer-atom-transfer radical polymerization (ARGET-ATRP; FIG. 3A); and a GPC chromatogram showing overlay of DPPE-dm-pMPC batch LS1, polymerized via ATRP using a DPPE-dm-Br (Procedure 1) and DPPE-dm-pMPC30 polymerized via ARGET-ATRP using a DPPE-dm-Br (Procedure 2)(FIG. 3B).

FIGS. 4A-B are schemes presenting an exemplary two-step procedure (Procedure 2) of preparing a phenylated brominated derivatized-DPPE initiator (DPPE-Ph-Br; FIG. 4A) and the polymerization of MPC from the DPPE-Ph-Br initiator via ARGET-ATRP to provide the LPC DPPE-Ph-pMPC (FIG. 4B).

FIG. 4C-D present GPC chromatograms showing overlay of DPPE-Ph-pMPC preparing using DPPE-Ph-Br via ARGET-ATRP (Procedure 2, batch 1056; dashed plot) and of DPPE-dm-pMPC30 polymerized via ARGET-ATRP using DPPE-dm-Br (Procedure 2, dotted plot)(FIG. 4C); and overlay of DPPE-Ph-pMPC preparing using DPPE-Ph-Br via ARGET-ATRP (Procedure 2, batch 1056; dashed plot) and of DPPE-dm-pMPC batch LS1, polymerized via ATRP using a DPPE-dm-Br (Procedure 1, LS1; solid plot)(FIG. 4D).

FIGS. 5A-B are schemes presenting an exemplary two-step synthesis of a bis-thiolated brominated initiator (2C16S-Prop-Br) from propargyl alcohol (FIG. 5A) and the LPC 2C16S-Prop-pMPC (FIG. 5B), obtained following polymerization of MPC using the exemplary 2C16S-Prop-Br initiator.

FIGS. 6A-B are schemes presenting an exemplary two-step synthesis of a bis-palmitoyl brominated initiator (2C16-TMP-Br) from trimethylolpropane (TMP)(FIG. 6A) and the LPC 2C16-TMP-pMPC (FIG. 6B), obtained following polymerization of MPC using the exemplary 2C16-TMP-Br initiator.

FIGS. 7A-C present DSC thermograms of liposome series with 0.7% exemplary liposome samples comprising long (_L) and short(S) LPCs (DPPE-dm-pMPC (DM), DPPE-Ph-pMPC (Ph), 2C16S-Prop-pMPC (Prop), 2C16-TMP-pMPC (TMP); FIG. 7A), and for different length of Propargyl-based polymers (Prop_S and Prop_L; FIG. 7B) and TMP-based polymers (TMP_S and TMP_L; FIG. 7C), all prepared by Procedure 2 as described herein.

FIGS. 8A-D are images from CryoTEM analyses of exemplary liposome samples with 3.5% of DPPE-Ph-pMPC_S (FIG. 8A; scale bar is 100 nm); DPPE-Ph-pMPC_L (FIG. 8B; scale bar is 0.2 μm); 2C16-TMP-pMPC_S (FIG. 8C; scale bar is 0.2 μm); and 2C16-TMP-pMPC_L (FIG. 8D; scale bar is 0.2 μm).

FIG. 9 is bar graph showing % viability following 72 hours incubation of L929 mouse cells with exemplary liposome samples comprising long (_L) and short (_S) LPCs (DPPE-dm-pMPC (DM), DPPE-Ph-pMPC (Ph), 2C16S-Prop-pMPC (Prop), 2C16-TMP-pMPC (TMP)) in comparison with two 0.7% short DPPE-dm-pMPC (0.7% DM_S) samples after a one-year storage (marked 1-year storage A and B), in a cytotoxicity assay. The horizontal line marks 70% viability.

FIGS. 10A-B are bar graphs showing C-activation by liposomes containing different types of LPC.

FIG. 11 presents comparative plots showing the Zeta potential (ZP) of pMPC as function of pH. Zeta potentials were measured for liposomes composed of DSPC, a zwitterionic lipid, and LPC (blue circles) and for a water soluble pMPC polymer (orange squares).

FIG. 12 presents comparative plots showing the Zeta potential of liposome formulations in low salt solution. The curves reflect both the increasing LPC membrane content and the dependence on LPC size.

FIGS. 13A-B present comparative plots showing the Zeta potential as a function of the total ion concentration for liposome formulations incorporating a short LPC (FIG. 13A) and a long LPC (FIG. 13B). For the formulations with the long LPC, the ZP decayed very quickly with salt and therefore ZP measurements at higher salts were not applicable. Dashed lines are fits it a linear equation. The slope is the of LPC layer thickness.

FIG. 14 presents comparative plots showing the mean LPC layer thickness as a function of the LPC length, at a range of concentrations. The increase rate in layer thickness for short LPC is lower than for long LPC. The thickness of the long LPC layer is 3-folds larger than for the short LPC.

FIGS. 15A-B presents comparative plots showing a correlation between liposome surface properties and the immunogenic response determined by complement activation related pseudoallergy (CARPA), for a short LPC (FIG. 15A) and a long LPC (FIG. 15B).

### DESCRIPTION OF SPECIFIC EMBODIMENTS OF THE INVENTION

The present invention, in some embodiments thereof, relates to material science and, more particularly, but not exclusively, to novel polymeric compounds usable, inter alia, for forming liposomes, and to uses of such liposomes in, for example, biomedical applications.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

As discussed hereinabove, WO 2017/109784 describes the design and preparation of polymeric compounds which bear phosphocholine analogs as pendant groups and are conjugated to a lipid moiety.

These polymeric compounds are capable of stabilizing lipid layers such as those of liposomes, while exhibiting considerably enhanced stability and effective lubrication between sliding surfaces, particularly in a saline environment (e.g., a physiological environment) and/or at high pressures.

In view of the highly advantageous properties of these lipid-containing polymeric compounds, which are also referred to herein as lipid-polymer conjugates, or LPC, the present inventors have sought for improved methodologies for preparing such LPCs. As discussed in further detail in the Examples section that follows, the present inventors have designed and successfully prepared novel synthetic approaches for preparing LPCs, and novel LPCs prepared thereby. More specifically, the present inventors have designed and successfully practiced both a novel synthetic methodology and newly designed lipid-containing compounds that are used for preparing the LPC, which provide an improved control of the polymeric portion of the LPC and LPCs that feature improved performance.

Embodiments of the present invention therefore relate to newly designed polymeric compounds which bear phosphocholine analogs as pendant groups and are conjugated to a lipid (e.g., phospholipid) moiety. Exemplary such polymeric compounds are represented by Formula I. These polymeric compounds are also referred to herein as “lipid-containing polymeric compounds” or simply as “polymeric compounds”, or as “lipid-polymer conjugates” or by the abbreviation “LPCs”.

The lipid-containing polymeric compounds disclosed herein are capable of stabilizing liposomes used for various applications (including in vivo applications) against aggregation and fusion, thereby increasing shelf life, while retaining and even enhancing properties associated with the surfaces of liposomes and other phospholipid layers, such as biocompatibility, and lubricant activity (e.g., by hydration lubrication). The disclosed polymeric compounds per se are also capable of forming stable micelles in an aqueous environment which can be used as stable replacement for liposomes in various applications (including in vivo applications), such as lubrication, including lubrication of interfaces with physiological surfaces. The disclosed polymeric compounds and/or liposomes formed therewith are also usable as drug delivery vehicles, in ophthalmic applications, and in other uses, as described herein.

The present inventors have uncovered that liposomes made of the newly designed polymeric compounds exhibit substantially reduced immunogenicity, also when formed of a negatively-charged bilayer-forming lipids.

The present inventors have further uncovered that lipid bilayers (e.g., in a form of liposomes) that comprise the newly designed polymeric compounds as described herein and optionally a negatively-charged bilayer-forming lipid and/or a sterol such as cholesterol, can be efficiently used in delivering therapeutically active agents to a subject's bodily site.

### Polymeric Compounds (LPCs):

According to an aspect of some embodiments of the invention, there are provided polymeric compounds collectively represented by Formula I:

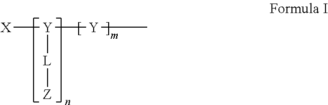

wherein:
m is zero or a positive integer;
n is an integer which is at least 2, at least 5, preferably at least 10 (e.g., of from 10 to 200);
Y is a backbone unit which forms a polymeric backbone of the polymeric compound;
X is a lipid moiety as described herein in any of the respective embodiments;
L is absent or is a linking moiety; and
Z has the general Formula II:

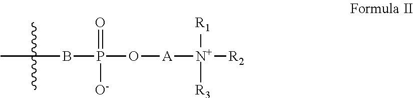

wherein:
the dashed (curved) line denotes an attachment point to the respective Y backbone unit or to the linking moiety L, if present;
A is a substituted or unsubstituted hydrocarbon;
B is an oxygen atom or is absent; and
R₁-R₃ are each independently selected from the group consisting of hydrogen, alkyl, cycloalkyl, heteroalicyclic, aryl and heteroaryl,
as described in more detail herein below.

Formula I may also be described herein simply as:

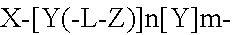

which is to be regarded as interchangeable with the schematic depiction hereinabove, wherein X is a lipid moiety conjugated to the —[Y(-L-Z)]n[Y]m- polymeric moiety.

### Polymeric Moiety:

Herein, the term “polymeric” refers to a compound having at least 2 repeating units (and more preferably at least 3 repeating units), the repeating units being identical or similar. It is to be appreciated that the compound of general Formula I is by definition polymeric when n is at least 2, as it comprises at least 2 of the backbone units represented by Y.

Herein, the phrase “polymeric moiety” refers to the portion of the polymeric compound (according to any of the embodiments described herein relating to general Formula I) which has the general Formula Ia:

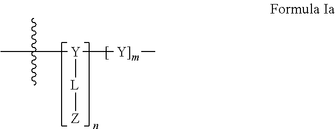

wherein m, n, Y, L and Z are as defined herein for general Formula I, and the dashed (curved) line represents an attachment point to the X lipid moiety.

Formula Ia may also be described herein simply as:

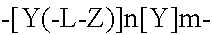

which is to be regarded as interchangeable with the schematic depiction hereinabove.

Herein, the phrase “polymeric compound” further encompasses compounds having a “polymeric moiety” as described herein having at least one unit (e.g., according to Formula Ia wherein n is at least 1), provided that the lipid moiety described herein (e.g., the lipid moiety represented by X) has a similar unit. For example, when the lipid moiety comprises a phosphate group (e.g., the lipid moiety is a glycerophospholipid moiety), and a single unit of the polymeric moiety has a phosphate group, the two phosphate groups may be regarded as repeating units.

In preferred embodiments, however, n is at least 2, such that the polymeric moiety per se has at least two units. In some embodiments, n is at least 3.

As used herein, the term “backbone unit” refers to a repeating unit, wherein linkage of a plurality of the repeating unit (e.g., sequential linkage) forms a polymeric backbone. A plurality of linked repeating units per se is also referred to herein as a “polymeric backbone”. A polymeric moiety as described herein can comprise a plurality of repeating backbone units which identical to one another, and thereby form a homopolymeric moiety, or, alternatively, can comprise two or more types of repeating backbone units, which can be linked to one another randomly or in a certain order (e.g., as two or more blocks, or in alternating order), and thereby form a copolymer moiety.

As shown in Formulae I and Ia, L and Z together form a pendant group of at least a portion of the backbone units, which group is referred to herein for brevity simply as the “pendant group”.

Each backbone unit Y with a pendant group (i.e., a unit represented by Y(-L-Z), the number of which is represented by the variable n) and each backbone unit Y without a pendant group (the number of which is represented by the variable m) is also referred to herein as a “monomeric unit”.

A backbone unit may optionally be a unit of a polymerizable monomer or polymerizable moiety of a monomer. A wide variety of polymerizable monomers and moieties will be known to the skilled person, and the structure of the units of such monomers which result upon polymerization (e.g., monomeric units) will also be known to the skilled person.

A “unit of a polymerizable monomer” refers to a modified form of a polymerizable monomer and/or a portion of a polymerizable monomer that remains after polymerization.

A portion of a polymerizable monomer may be formed, for example, by a condensation reaction, e.g., wherein at least one atom or group (e.g., a hydrogen atom or hydroxyl group) in the monomer, and optionally at least two atoms or groups (e.g., a hydrogen atom and a hydroxyl group) in the monomer, is replaced with a covalent bond with another polymerizable monomer.

A modified form of a polymerizable monomer may be formed, for example, by ring-opening (wherein a covalent bond between two atoms in a ring is broken, and each of the two atoms optionally becomes linked to another polymerizable monomer); and/or by adding to an unsaturated bond, wherein an unsaturated bond between two adjacent atoms is broken (e.g., conversion of an unsaturated double bond to a saturated bond, or conversion of an unsaturated triple bond to an unsaturated double bond) and the two atoms optionally each become linked to another polymerizable monomer.

A modified form of a polymerizable monomer may consist essentially of the same atoms as the original monomer, for example, different merely in the rearrangement of covalent bonds, or alternatively, may have a different atomic composition, for example, wherein polymerization includes a condensation reaction (e.g., as described herein).

Examples of backbone units include, without limitation, substituted or unsubstituted hydrocarbons (which may form a substituted or unsubstituted hydrocarbon backbone), such as alkylene units; hydroxycarboxylic acid units (which may form a polyester backbone), e.g., glycolate, lactate, hydroxybutyrate, hydroxyvalerate, hydroxycaproate and hydroxybenzoate units; dicarboxylic acid units (which may form a polyester backbone in combination with a diol and/or a polyamide in combination with a diamine), e.g., adipate, succinate, terephthalate and naphthalene dicarboxylic acid units; diol units (which may form a polyether backbone, or form a polyester backbone in combination with a dicarboxylic acid), e.g., ethylene glycol, 1,2-propanediol, 1,3-propanediol, 1,4-butanediol, and bisphenol A units; diamine units (which may form a polyamide backbone in combination with a dicarboxylic acid), e.g., para-phenylene diamine and alkylene diamines such hexylene diamine; carbamate units (which may form a polyurethane backbone); amino acid residues (which may form a polypeptide backbone); and saccharide moieties (which may form a polysaccharide backbone).

In some embodiments of any of the embodiments described herein, Y is a substituted or unsubstituted alkylene unit.

In some embodiments, Y is a substituted or unsubstituted ethylene unit, that is, an alkylene unit 2 atoms in length.

Polymeric backbones wherein Y is a substituted or unsubstituted ethylene unit may optionally be a polymeric backbone such as formed by polymerizing ethylene (CH₂═CH₂) and/or substituted derivatives thereof (also referred to herein as “vinyl monomers”). Such polymerization is a very well-studied procedure, and one of ordinary skill in the art will be aware of numerous techniques for effecting such polymerization.

It is to be understood that any embodiments described herein relating to a polymeric backbone formed by a polymerization encompass any polymeric backbone having a structure which can be formed by such polymerization, regardless of whether the polymeric backbone was formed in practice by such polymerization (or any other type of polymerization).

As is well known in the art, the unsaturated bond of ethylene and substituted ethylene derivatives becomes saturated upon polymerization, such that the backbone units in a polymeric backbone formed by the polymerization are saturated, although they may be referred to as units of an unsaturated compound (e.g., a “vinyl monomer” or “olefin monomer”) to which they are analogous.

Polymers which can be formed from unsaturated monomers such as vinyl monomers and olefin monomers are also referred to by the terms “polyvinyl” and “polyolefin”, respectively.

Herein, an “unsubstituted” alkylene unit (e.g., ethylene unit) refers to an alkylene unit which does not have any substituent other than the pendant group discussed herein (represented as (-L-Z)). That is, an alkylene unit attached to the aforementioned pendant group is considered unsubstituted if there are no substituents at any other positions on the alkylene unit.

In some embodiments of any of the embodiments described herein, Y has the formula —CR₄R₅—CR₆D-.

When Y is a backbone unit which is not attached to L or Z (i.e., to a pendant group as described herein), D is R₇ (an end group, as defined herein); and when Y is a backbone unit which is attached to L or Z, D is a covalent bond or a linking group attaching Y to L or Z. The linking group may optionally be —O—, —S—, arylene, sulfinyl, sulfonyl, phosphate, phosphonyl, phosphinyl, carbonyl, thiocarbonyl, urea, thiourea, O-carbamyl, N-carbamyl, O-thiocarbamyl, N-thiocarbamyl, C-amido, N-amido, C-carboxy, O-carboxy, sulfonamido, or amino.

R₄-R₇ are each independently hydrogen, alkenyl, alkynyl, cycloalkyl, aryl, heteroaryl, heteroalicyclic, halo, hydroxy, alkoxy, aryloxy, thiohydroxy, thioalkoxy, thioaryloxy, sulfinyl, sulfonyl, cyano, nitro, azide, azo, phosphate phosphonyl, phosphinyl, oxo, carbonyl, thiocarbonyl, urea, thiourea, O-carbamyl, N-carbamyl, O-thiocarbamyl, N-thiocarbamyl, C-amido, N-amido, C-carboxy, O-carboxy, sulfonamido, or amino.

Herein throughout, the phrase “linking group” describes a group (e.g., a substituent) that is attached to two or more moieties in the compound.

Herein throughput, the phrase “end group” describes a group (e.g., a substituent) that is attached to a single moiety in the compound via one atom thereof.

When each of R₄-R₆ is hydrogen, and D is a covalent bond or linking group, Y is an unsubstituted ethylene group attached (via D) to a pendant group described herein.

When each of R₄-R₇ is hydrogen (and D is R₇), Y is an unsubstituted ethylene group which is not attached to a pendant group described herein.

In some embodiments of any of the embodiments described herein, R₄ and R₅ are each hydrogen. Such embodiments include polymeric backbones formed from many widely used vinyl monomers (including ethylene), including, for example, olefins (e.g., ethylene, propylene, 1-butylene, isobutylene, 4-methyl-1-pentene), vinyl chloride, styrene, vinyl acetate, acrylonitrile, acrylate and derivatives thereof (e.g., acrylate esters, acrylamides), and methacrylate and derivatives thereof (e.g., methacrylate esters, methacrylamides).

In some embodiments of any of the embodiments described herein, R₆ is hydrogen. In some such embodiments, R₄ and R₅ are each hydrogen.

In some embodiments of any of the embodiments described herein, R₆ is methyl. In some such embodiments, R₄ and R₅ are each hydrogen. In some such embodiments, the backbone unit is a unit of methacrylate or a derivative thereof (e.g., methacrylate ester, methacrylamide).

In some embodiments of any of the embodiments described herein, the linking group represented by the variable D is —O—, —C(═O)O—, —C(═O)NH— or phenylene. In exemplary embodiments, D is —C(═O)O—.

For example, the backbone unit may optionally be a vinyl alcohol derivative (e.g., an ester or ether of a vinyl alcohol unit) when Dis —O—; an acrylate or methacrylate derivative (e.g., an ester of an acrylate or methacrylate unit) when D is —C(═O)O—; an acrylamide or methacrylamide unit when D is —C(═O)NH—; and/or a styrene derivative (e.g., a substituted styrene unit) when D is phenylene.

In some embodiments of any of the embodiments described herein, L is a substituted or unsubstituted hydrocarbon from 1 to 10 carbon atoms in length. In some embodiments, the hydrocarbon is unsubstituted. In some embodiments, the hydrocarbon is a linear, unsubstituted hydrocarbon, that is, —(CH₂)ᵢ— wherein i is an integer from 1 to 10.

In some embodiments of any of the embodiments described herein, L is a substituted or unsubstituted ethylene group. In some embodiments, L is an unsubstituted ethylene group (—CH₂CH₂—).

In some embodiments of any of the embodiments described herein, B is an oxygen atom. In some such embodiments, L is a hydrocarbon according to any of the respective embodiments described herein (i.e., L is not absent), and Z is a phosphate group attached to L.

In some embodiments of any of the embodiments described herein, B is absent. In some such embodiments, L is a hydrocarbon according to any of the respective embodiments described herein (i.e., L is not absent), and Z is a phosphonate group attached to L. In some embodiments, L is also absent, such that the phosphorus atom of Formula II is attached directly to Y.

In some embodiments of any of the embodiments described herein, A is a substituted or unsubstituted hydrocarbon from 1 to 4 carbon atoms in length.

In some embodiments of any of the embodiments described herein, A is an unsubstituted hydrocarbon. In some such embodiments, the unsubstituted hydrocarbon is from 1 to 4 carbon atoms in length. In some embodiments, the hydrocarbon is a linear, unsubstituted hydrocarbon, that is, —(CH₂)ⱼ— wherein j is an integer from 1 to 4.

In some embodiments of any of the embodiments described herein, A is a substituted or unsubstituted ethylene group.

In some embodiments of any of the embodiments described herein, A is an unsubstituted ethylene group (—CH₂CH₂—). In such embodiments, the moiety having general Formula II (represented by the variable Z) is similar or identical to a phosphoethanolamine or phosphocholine moiety. Phosphoethanolamine and phosphocholine moieties are present in many naturally occurring compounds (e.g., phosphatidylcholines, phosphatidylethanolamines).

In some embodiments of any of the embodiments described herein, A is an ethylene group substituted by a C-carboxy group. In some embodiments, the C-carboxy is attached to the carbon atom adjacent to the nitrogen atom depicted in Formula II (rather than the carbon atom attached to the depicted oxygen atom). In such embodiments, the moiety having general Formula II (represented by the variable Z) is similar or identical to a phosphoserine moiety. Phosphoserine is present in many naturally occurring compounds (e.g., phosphatidylserines).

Without being bound by any particular theory, it is believed that moieties similar or identical to naturally occurring moieties such as phosphocholine, phosphoethanolamine and/or phosphoserine may be particularly biocompatible.

In some embodiments of any of the embodiments described herein, R₁-R₃ (the substituents of the nitrogen atom depicted in general Formula II) are each independently hydrogen or C₁₋₄-alkyl. In some embodiments, R₁-R₃ are each independently hydrogen or methyl. In some embodiments, R₁-R₃ are each methyl. In some such embodiments, R₁-R₃ are each hydrogen.

The variable n may be regarded as representing a number of backbone units (represented by the variable Y) which are substituted by the pendant group represented by (-L-Z), and the variable m may be regarded as representing a number of backbone units which are not substituted by such a pendant group. The sum n+m may be regarded as representing the total number of backbone units in the polymeric backbone. The ratio n/(n+m) may be regarded as representing the fraction of backbone units which are substituted by the pendant group represented by (-L-Z).

In some embodiments of any of the embodiments described herein, the percentage of backbone units (represented by the variable Y) which are substituted by the pendant group represented by (-L-Z)(as represented by the formula 100%*n/(n+m)) is at least 20%. In some embodiments, the percentage of backbone units substituted by the aforementioned pendant group is at least 30%. In some embodiments, the percentage of backbone units substituted by the aforementioned pendant group is at least 40%. In some embodiments, the percentage of backbone units substituted by the aforementioned pendant group is at least 50%. In some embodiments, the percentage of backbone units substituted by the aforementioned pendant group is at least 60%. In some embodiments, the percentage of backbone units substituted by the aforementioned pendant group is at least 70%. In some embodiments, the percentage of backbone units substituted by the aforementioned pendant group is at least 80%. In some embodiments, the percentage of backbone units substituted by the aforementioned pendant group is at least 90%. In some embodiments, the percentage of backbone units substituted by the aforementioned pendant group is at least 95%. In some embodiments, the percentage of backbone units substituted by the aforementioned pendant group is at least 98%.

In some embodiments of any of the embodiments described herein, m is 0, such that each of the backbone units (represented by the variable Y) is substituted by the pendant group represented by (-L-Z).

In some embodiments of any of the embodiments described herein, n is at least 5. In some embodiments, n is at least 10. In some embodiments, n is at least 15.

In some embodiments of any of the embodiments described herein, n is in a range of from 2 to 1,000, including any intermediate value and subranges therebetween. In some embodiments of any of the embodiments described herein, n is in a range of from 2 to 500, including any intermediate value and subranges therebetween. In some embodiments of any of the embodiments described herein, n is in a range of from 2 to 200, including any intermediate value and subranges therebetween. In some embodiments of any of the embodiments described herein, n is in a range of from 2 to 100, including any intermediate value and subranges therebetween. In some embodiments of any of the embodiments described herein, n is in a range of from 2 to 50, including any intermediate value and subranges therebetween. In some such embodiments, m is 0.

In some embodiments of any of the embodiments described herein, n is in a range of from 3 to 1,000, including any intermediate value and subranges therebetween. In some embodiments of any of the embodiments described herein, n is in a range of from 3 to 500, including any intermediate value and subranges therebetween. In some embodiments of any of the embodiments described herein, n is in a range of from 3 to 200, including any intermediate value and subranges therebetween. In some embodiments of any of the embodiments described herein, n is in a range of from 3 to 100, including any intermediate value and subranges therebetween. In some embodiments of any of the embodiments described herein, n is in a range of from 3 to 50, including any intermediate value and subranges therebetween. In some embodiments of any of the embodiments described herein, n is in a range of from 5 to 50, including any intermediate value and subranges therebetween. In some embodiments of any of the embodiments described herein, n is in a range of from 10 to 50, including any intermediate value and subranges therebetween. In some embodiments of any of the embodiments described herein, n is in a range of from 10 to 25, including any intermediate value and subranges therebetween. In some such embodiments, m is 0.

In some embodiments of any of the embodiments described herein, n is in a range of from to 200, including any intermediate value and subranges therebetween. In some embodiments of any of the embodiments described herein, n is in a range of from 10 to 180, including any intermediate value and subranges therebetween. In some embodiments of any of the embodiments described herein, n is in a range of from 10 to 150, including any intermediate value and subranges therebetween. In some embodiments of any of the embodiments described herein, n is in a range of from 10 to 120, including any intermediate value and subranges therebetween 0. In some such embodiments, m is 0.

In some embodiments of any of the embodiments described herein, n is at least 30.

In some embodiments of any of the embodiments described herein, n is in a range of from 30 to 200, including any intermediate value and subranges therebetween. In some embodiments of any of the embodiments described herein, n is in a range of from 30 to 180, including any intermediate value and subranges therebetween. In some embodiments of any of the embodiments described herein, n is in a range of from 30 to 150, including any intermediate value and subranges therebetween. In some embodiments of any of the embodiments described herein, n is in a range of from 30 to 120, including any intermediate value and subranges therebetween 0. In some such embodiments, m is 0.

In some embodiments of any of the embodiments described herein, n is in a range of from 30 to 70, including any intermediate value and subranges therebetween. In some embodiments of any of the embodiments described herein, n is in a range of from 35 to 65, including any intermediate value and subranges therebetween. In some such embodiments, m is 0.

In some embodiments of any of the embodiments described herein, n is at least 50, or at least 60, or at least 80.

In some embodiments of any of the embodiments described herein, n is in a range of from 50 to 200, including any intermediate value and subranges therebetween. In some embodiments of any of the embodiments described herein, n is in a range of from 50 to 180, including any intermediate value and subranges therebetween. In some embodiments of any of the embodiments described herein, n is in a range of from 50 to 150, including any intermediate value and subranges therebetween. In some embodiments of any of the embodiments described herein, n is in a range of from 50 to 120, including any intermediate value and subranges therebetween. In some embodiments of any of the embodiments described herein, n is in a range of from 80 to 120, including any intermediate value and subranges therebetween. In some embodiments of any of the embodiments described herein, n is in a range of from 50 to 100, including any intermediate value and subranges therebetween. In some embodiments of any of the embodiments described herein, n is in a range of from 50 to 80, including any intermediate value and subranges therebetween. In some such embodiments, m is 0.

In some embodiments of any of the embodiments described herein, when n is lower than 80, or lower than 70, or lower than 50, or lower than 30, or is in a range of from 10 to 50, or from 30 to 60, or from 30 to 80, or from 30 to 70, or from 50 to 80, as described herein in any of the respective embodiments, the polymeric compound is referred to herein as “short” or “S”.

In some embodiments of any of the embodiments described herein, when n is higher than 80, or is higher than 100, or is in a range of from 50 to 150, or from 50 to 120, or from 80 to 150, or from 80 to 120, as described herein in any of the respective embodiments, the polymeric compound is referred to herein as “long” or “L”.

In some embodiments of any of the embodiments described herein, n is in a range of from 10 to 50, including any intermediate value and subranges therebetween. In some such embodiments, m is 0.

In some embodiments of any of the embodiments described herein, n is in a range of from 50 to 80, including any intermediate value and subranges therebetween. In some such embodiments, m is 0.

In some embodiments of any of the embodiments described herein, n is in a range of from 80 to 120, including any intermediate value and subranges therebetween. In some such embodiments, m is 0.

In some embodiments of any of the embodiments described herein, m is in a range of from 0 to 1,000, including any intermediate value and subranges therebetween. In some such embodiments, n is in a range of from 2 to 1,000, including any intermediate value and subranges therebetween, such that the total number of backbone units is in a range of from 2 to 2,000, including any intermediate value and subranges therebetween. In some such embodiments, n is in a range of from 3 to 1,000, including any intermediate value and subranges therebetween. In some embodiments, n is in a range of from 3 to 500, including any intermediate value and subranges therebetween. In some embodiments, n is in a range of from 3 to 200, including any intermediate value and subranges therebetween. In some embodiments, n is in a range of from 3 to 100, including any intermediate value and subranges therebetween. In some embodiments, n is in a range of from 3 to 50, including any intermediate value and subranges therebetween. In some embodiments, n is in a range of from 5 to 50, including any intermediate value and subranges therebetween. In some embodiments, n is in a range of from 10 to 50, including any intermediate value and subranges therebetween. In some embodiments, n is in a range of from 50 to 80, including any intermediate value and subranges therebetween. In some embodiments, n is in a range of from 80 to 120, including any intermediate value and subranges therebetween.

In some embodiments of any of the embodiments described herein, m is in a range of from 0 to 500, including any intermediate value and subranges therebetween. In some such embodiments, n is in a range of from 2 to 1,000, including any intermediate value and subranges therebetween. In some such embodiments, n is in a range of from 3 to 1,000, including any intermediate value and subranges therebetween. In some embodiments, n is in a range of from 3 to 500, including any intermediate value and subranges therebetween. In some embodiments, n is in a range of from 3 to 200, including any intermediate value and subranges therebetween. In some embodiments, n is in a range of from 3 to 100, including any intermediate value and subranges therebetween. In some embodiments, n is in a range of from 3 to 50, including any intermediate value and subranges therebetween. In some embodiments, n is in a range of from 5 to 50, including any intermediate value and subranges therebetween. In some embodiments, n is in a range of from 10 to 50, including any intermediate value and subranges therebetween. In some embodiments, n is in a range of from 50 to 80, including any intermediate value and subranges therebetween. In some embodiments, n is in a range of from 80 to 120, including any intermediate value and subranges therebetween.

In some embodiments of any of the embodiments described herein, m is in a range of from 0 to 200, including any intermediate value and subranges therebetween. In some such embodiments, n is in a range of from 2 to 1,000, including any intermediate value and subranges therebetween. In some such embodiments, n is in a range of from 3 to 1,000, including any intermediate value and subranges therebetween. In some embodiments, n is in a range of from 3 to 500, including any intermediate value and subranges therebetween. In some embodiments, n is in a range of from 3 to 200, including any intermediate value and subranges therebetween. In some embodiments, n is in a range of from 3 to 100, including any intermediate value and subranges therebetween. In some embodiments, n is in a range of from 3 to 50, including any intermediate value and subranges therebetween. In some embodiments, n is in a range of from 5 to 50, including any intermediate value and subranges therebetween. In some embodiments, n is in a range of from 10 to 50, including any intermediate value and subranges therebetween. In some embodiments, n is in a range of from 50 to 80, including any intermediate value and subranges therebetween. In some embodiments, n is in a range of from 80 to 120, including any intermediate value and

In some embodiments of any of the embodiments described herein, m is in a range of from 0 to 100, including any intermediate value and subranges therebetween. In some such embodiments, n is in a range of from 2 to 1,000, including any intermediate value and subranges therebetween. In some such embodiments, n is in a range of from 3 to 1,000, including any intermediate value and subranges therebetween. In some embodiments, n is in a range of from 3 to 500. In some embodiments, n is in a range of from 3 to 200, including any intermediate value and subranges therebetween. In some embodiments, n is in a range of from 3 to 100, including any intermediate value and subranges therebetween. In some embodiments, n is in a range of from 3 to 50. In some embodiments, n is in a range of from 5 to 50, including any intermediate value and subranges therebetween. In some embodiments, n is in a range of from 10 to 50, including any intermediate value and subranges therebetween. In some embodiments, n is in a range of from 50 to 80, including any intermediate value and subranges therebetween. In some embodiments, n is in a range of from 80 to 120, including any intermediate value and subranges therebetween.

In some embodiments of any of the embodiments described herein, m is in a range of from 0 to 50, including any intermediate value and subranges therebetween. In some such embodiments, n is in a range of from 2 to 1,000, including any intermediate value and subranges therebetween. In some such embodiments, n is in a range of from 3 to 1,000, including any intermediate value and subranges therebetween. In some embodiments, n is in a range of from 3 to 500, including any intermediate value and subranges therebetween. In some embodiments, n is in a range of from 3 to 200, including any intermediate value and subranges therebetween. In some embodiments, n is in a range of from 3 to 100, including any intermediate value and subranges therebetween. In some embodiments, n is in a range of from 3 to 50, including any intermediate value and subranges therebetween. In some embodiments, n is in a range of from 5 to 50, including any intermediate value and subranges therebetween. In some embodiments, n is in a range of from 10 to 50, including any intermediate value and subranges therebetween. In some embodiments, n is in a range of from 50 to 80, including any intermediate value and subranges therebetween. In some embodiments, n is in a range of from 80 to 120, including any intermediate value and subranges therebetween.

In some embodiments of any of the embodiments described herein, m is in a range of from 0 to 20, including any intermediate value and subranges therebetween. In some such embodiments, n is in a range of from 2 to 1,000, including any intermediate value and subranges therebetween. In some such embodiments, n is in a range of from 3 to 1,000, including any intermediate value and subranges therebetween. In some embodiments, n is in a range of from 3 to 500, including any intermediate value and subranges therebetween. In some embodiments, n is in a range of from 3 to 200, including any intermediate value and subranges therebetween. In some embodiments, n is in a range of from 3 to 100, including any intermediate value and subranges therebetween. In some embodiments, n is in a range of from 3 to 50, including any intermediate value and subranges therebetween. In some embodiments, n is in a range of from 5 to 50, including any intermediate value and subranges therebetween. In some embodiments, n is in a range of from 10 to 50, including any intermediate value and subranges therebetween. In some embodiments, n is in a range of from 50 to 80, including any intermediate value and subranges therebetween. In some embodiments, n is in a range of from 80 to 120, including any intermediate value and subranges therebetween.

In some embodiments of any of the embodiments described herein, m is in a range of from 0 to 10, including any intermediate value and subranges therebetween. In some such embodiments, n is in a range of from 2 to 1,000, including any intermediate value and subranges therebetween. In some such embodiments, n is in a range of from 3 to 1,000, including any intermediate value and subranges therebetween. In some embodiments, n is in a range of from 3 to 500, including any intermediate value and subranges therebetween. In some embodiments, n is in a range of from 3 to 200, including any intermediate value and subranges therebetween. In some embodiments, n is in a range of from 3 to 100, including any intermediate value and subranges therebetween. In some embodiments, n is in a range of from 3 to 50, including any intermediate value and subranges therebetween. In some embodiments, n is in a range of from 5 to 50, including any intermediate value and subranges therebetween. In some embodiments, n is in a range of from 10 to 50, including any intermediate value and subranges therebetween. In some of any of the embodiments described herein for m, n is in a range of from 30 to 70, as described herein in any of the respective embodiments, or represents a short polymeric moiety, as described herein. In some such embodiments, n is in a range of from 50 to 80, including any intermediate value and subranges therebetween.

In some of any of the embodiments described herein for m, n is in a range of from 80 to 120, as described herein in any of the respective embodiments, or represents a long polymeric moiety, as described herein.

In some embodiments of any of the embodiments described herein, the backbone unit Y which is substituted by the pendant group represented by (-L-Z) is the same as the backbone unit Y which is not substituted by the pendant group (e.g., when m is at least 1). In alternative embodiments, at least a portion of the backbone units Y which are substituted by the pendant group are different than a portion of the backbone unit Y which is not substituted by the pendant group (e.g., when m is at least 1).

In some embodiments of any of the embodiments described herein, the plurality (indicated by the variable n) of backbone units Y which are substituted by the pendant group represented by (-L-Z) are the same as each other. In alternative embodiments, at least a portion of the plurality of backbone units Y which are substituted by the pendant group represented by (-L-Z) are different from a second portion of the plurality of backbone units Y which are substituted by the pendant group.

In some embodiments of any of the embodiments described herein, the plurality (indicated by the variable n) of pendant groups (-L-Z) attached to the plurality of backbone units Y are the same as each other. In alternative embodiments, at least a portion of the pendant groups (-L-Z) attached to the plurality of backbone units Y are different from each other (e.g., differ in the identity of any one or more of A, B, R₁, R₂, R₃ and L).

In any of the embodiments described herein wherein more than one backbone unit Y is not substituted by the pendant group described herein (i.e., when m is greater than 1), the plurality (indicated by the variable m) of backbone units Y which are not substituted by the pendant group are the same as each other. In alternative embodiments wherein m is larger than 1, at least a portion of the backbone units Y which are not substituted by the pendant group described herein, are different from at least a second portion of the plurality of backbone units Y which are not substituted by the pendant group.

The number of types of backbone units substituted by the pendant group, the number of types of backbone units not substituted by the pendant group (if any such units are present), and/or the number of types of pendant group in the polymeric moiety may each independently be any number (e.g., 1, 2, 3, 4, 5, 6, 7, 8, 9, 10 or more).

In some embodiments of any of the embodiments described herein, the polymeric moiety is a copolymer moiety, that is, the polymeric moiety comprises at least two different types of monomeric unit. In some such embodiments, the different types of monomeric units differ in whether they comprise the pendant group (-L-Z) according to any of the respective embodiments described herein (e.g., when m is at least 1), and/or the different types of monomeric units differ in the type of backbone unit Y, and/or the different types of monomeric units differ in the type of pendant group (-L-Z).

For example, in some embodiments of any of the embodiments described herein the backbone unit Y in each of the Y(-L-Z) units may optionally be the same or different, while the L and Z moieties are the same among the Y(-L-Z) units. In some such embodiments, backbone units not substituted by the pendant group (if any such units are present) may optionally be the same as backbone unit Y in each of the Y(-L-Z) units. Alternatively, backbone units not substituted by the pendant group (if any such units are present) may optionally be different than backbone unit Y in each of the Y(-L-Z) units (while optionally being the same among all backbone units not substituted by the pendant group).

In some embodiments of any of the embodiments described herein the L moiety in each of the Y(-L-Z) units may optionally be the same or different, while the backbone units Y and the Z moieties are the same among the Y(-L-Z) units. In some such embodiments, backbone units not substituted by the pendant group (if any such units are present) may optionally be the same as backbone unit Y in each of the Y(-L-Z) units. Alternatively, backbone units not substituted by the pendant group (if any such units are present) may optionally be different than backbone unit Y in each of the Y(-L-Z) units (while optionally being the same among all backbone units not substituted by the pendant group).

In some embodiments of any of the embodiments described herein the Z moiety in each of the Y(-L-Z) units may optionally be the same or different, while the backbone units Y and the Z moieties are the same among the Y(-L-Z) units. In some such embodiments, backbone units not substituted by the pendant group (if any such units are present) may optionally be the same as backbone unit Y in each of the Y(-L-Z) units. Alternatively, backbone units not substituted by the pendant group (if any such units are present) may optionally be different than backbone unit Y in each of the Y(-L-Z) units (while optionally being the same among all backbone units not substituted by the pendant group).

In any of the embodiments described herein wherein the polymeric moiety is a copolymer moiety, any two or more different types of monomeric unit may be distributed randomly or non-randomly throughout the polymeric moiety. When different types of monomeric unit are distributed non-randomly, the copolymer may be one characterized by any non-random distribution, for example, an alternating copolymer, a periodic copolymer, and/or a block copolymer.

In some of any of the embodiments described herein, the polymeric moiety, which is attached at one of its termini to the lipid moiety X, can have various terminal groups at the other terminus (i.e., at the terminus near the backbone unit Y without a pendant group, wherein m is at least 1; or at the other terminus near the backbone unit Y with a pendant group, wherein m is zero).

The terminal group can be an intrinsic terminal group, derived from the monomers used to form the polymeric compound and/or from the process used to polymerize the monomers, or can be otherwise conjugated to, or generated within, the terminus of the polymeric moiety. For example, the terminal group can be hydrogen, halo, alkyl, hydroxy, carboxy and the like, or can be a targeting moiety, as described in further detail hereinafter. In some of any of the embodiments described herein, the terminal group is hydrogen or halo. In some of any of the embodiments described herein the terminal group is derived from the initiator used to form the polymeric compound as described herein in any of the respective embodiments and exemplified in the Examples section that follows, and in some of these embodiments the terminal group is a halo (e.g., chloro or bromo).

In some of any of the embodiments described herein, the terminal group is a functional group that is suitable for electron transfer radical polymerization, e.g., variable Ri in Formula V, as described herein in any of the respective embodiments.

### Lipid Moiety:

The lipid moiety (represented by the variable X in Formula I herein) according to any of the embodiments in this section may be attached to a polymeric moiety according to any of the embodiments described in the section herein relating to the polymeric moiety.

The lipid moiety may optionally be derived from any lipid known in the art (including, but not limited to, a naturally occurring lipid). Derivation of the lipid moiety from the lipid may optionally consist of substituting a hydrogen atom at any position of the lipid with the polymeric moiety represented in general Formula I by [Y(-L-Z)]n[Y]m (i.e., the polymeric moiety represented by general Formula Ia).

In some embodiments of any of the embodiments described herein, the lipid moiety (according to any of the respective embodiments described herein) is attached to a Y(-L-Z) unit (according to any of the embodiments described herein relating to Y, L and/or Z), that is, backbone unit substituted by the pendant group described herein (e.g., rather than a backbone unit not substituted by the pendant group).

Alternatively or additionally, in some embodiments of any of the embodiments described herein wherein m is at least 1, the lipid moiety (according to any of the respective embodiments described herein) may optionally be attached to a backbone unit (Y) which is not substituted by a pendant group described herein (e.g., rather than attached to a backbone unit substituted by the pendant group). For example, the polymeric moiety may optionally be a copolymer wherein the identity of the backbone unit attached to the lipid moiety varies randomly between molecules. Thus, the depiction of X in Formula I as being attached to a backbone unit substituted by a pendant group (i.e., Y-(L-Z)) rather than to an unsubstituted backbone unit Y is arbitrary, and is not intended to be limiting.

In some embodiments of any of the embodiments described herein, the lipid moiety is a moiety of a lipid which is a fatty acid, a monoglyceride, a diglyceride, a triglyceride, a glycerophospholipid, a sphingolipid, or a sterol. In some embodiments, the lipid is a glycerophospholipid.

In some embodiments of any of the embodiments described herein, the lipid moiety comprises at least one fatty acid moiety (e.g., an acyl group derived from a fatty acid). The fatty acid moiety may be derived from a saturated or unsaturated fatty acid. For example, the lipid moiety may consist of a fatty acid moiety, or be a monoglyceride moiety comprising one fatty acid moiety, a diglyceride moiety comprising two fatty acid moieties, or a triglyceride moiety comprising three fatty acid moieties.

Examples of fatty acid moieties which may optionally be comprised by the lipid moiety include, without limitation, lauroyl, myristoyl, palmitoyl, stearoyl, palmitoleoyl, oleoyl, and linoleoyl.

Suitable examples of glycerophospholipids include, without limitation, a phosphatidyl ethanolamine, a phosphatidyl serine, a phosphatidyl glycerol and a phosphatidyl inositol.

In some embodiments of any of the embodiments described herein, the lipid moiety represented by the variable X has the general Formula I is represented by Formula IV:

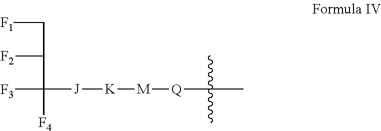

wherein:
the dashed (curved) line denotes an attachment point to the polymeric backbone (i.e., via the respective Y backbone unit);
F₁, F₂, F₃ and F₄ are each independently selected from the group consisting of hydrogen, alkyl, alkenyl, alkynyl, acyl, alkoxy, thioalkoxy, carboxy, thiocarboxy, wherein at least one of F₁, F₂, F₃ and F₄ is not hydrogen and is of at least 10 carbon atoms in length;
J is —O—P(═O)(OH)—O— or absent;
K is a substituted or unsubstituted hydrocarbon from 1 to 10 carbon atoms in length or absent;
M is a linking group selected from the group consisting of —O—, —S—, amino, sulfinyl, sulfonyl, phosphate, phosphonyl, phosphinyl, carbonyl, thiocarbonyl, urea, thiourea, carbamyl, thiocarbamyl, amido, carboxy, and sulfonamide, or absent; and
Q is a substituted or unsubstituted hydrocarbon from 1 to 10 carbon atoms in length, or absent,
wherein when M is absent, Q is also absent.
Q is attached to a backbone unit of the polymeric backbone according to any of the respective embodiments described herein, or alternatively, when Q is absent, M is attached to the aforementioned backbone unit.

When M is absent, Q is also absent, and K is attached to a backbone unit of the polymeric backbone according to any of the respective embodiments described herein.

In some embodiments of any of the embodiments described herein for Formula IV, when J is absent, M is not absent.

In some embodiments of any of the embodiments described herein for Formula IV, when J is —O—P(═O)(OH)—O—, M is other than amido and/or Q comprises an aryl moiety.

In some embodiments of any of the embodiments described herein for Formula IV, at least one of F₁, F₂, F₃ and F₄ is an alkoxy, thioalkoxy, acyl or carboxy, preferably of at least 10 carbon atoms in length, for example, of from 8 to 40, or of from 10 to 40, or of from 10 to 30, carbon atoms in length.

In some such embodiments, the alkoxy, thioalkoxy, acyl and/or carboxy has an alkyl moiety that is derived from a fatty acid acyl, as described herein, and is, for example, lauroyl, myristoyl, palmitoyl, stearoyl, palmitoleoyl, olcoyl, and linolcoyl.

In some embodiments of any of the embodiments described herein for Formula IV, at least one, or at least two, of F₁, F₂, F₃ and F₄ is independently a thioalkoxy. In some of these embodiments, the thioalkoxy is of at least 10 carbon atoms in length, for example, of from 8 to 40, or of from 10 to 40, or of from 10 to 30, carbon atoms in length. In exemplary embodiments, the alkyl is 15 carbon atoms in length and is derived from palmitic acid. In some of these embodiments, the thioalkoxy has an alkyl group that is of lauric acid, myristic acid, palmitic acid, stearic acid, oleic acid or linoleic acid. In some of these embodiments, each of F₁ and F₂ is independently a thioalkoxy as described herein, and can be the same or different, preferably the same. In some of these embodiments, F₃ and F₄ are each hydrogen.

In some embodiments of any of the embodiments described herein, at least one, or at least two, of F₁, F₂, F₃ and F₄ is independently a carboxy. In some of these embodiments, the carboxy is of at least 10 carbon atoms in length, for example, of from 8 to 40, or of from 10 to 40, or of from 10 to 30, carbon atoms in length. In exemplary embodiments, the carboxy is 16 carbon atoms in length and is derived from palmitic acid. In some of these embodiments, the carboxy has an alkyl group that is of lauric acid, myristic acid, palmitic acid, stearic acid, oleic acid or linoleic acid. In some of these embodiments, at least one or both of F₁ and F₂ is a carboxy as described herein in any of the respective embodiments. In some of these embodiments, both of F₁ and F₂ is a carboxy as described herein in any of the respective embodiments, which can be the same or different and is preferably the same. In some of any of these embodiments, at least one of F₃ and F₄ is an alkyl, which can be the same or different. In some such embodiments, the alkyl is a short alkyl of 1 to 6, or 1 to 4 carbon atoms in length, for example, methyl. Alternatively, each of F₃ and F₄ is hydrogen.

According to some of any of the embodiments described herein, M is other than amido.

According to some of any of the embodiments described herein, M is carboxy.

According to some of any of the embodiments described herein, M is carboxy and at least one, or at least two, of F₁, F₂, F₃ and F₄ is independently a thioalkoxy. In some of these embodiments, the thioalkoxy is of at least 10 carbon atoms in length, for example, of from 8 to 40, or of from 10 to 40, or of from 10 to 30, carbon atoms in length. In exemplary embodiments, the alkyl is 15 carbon atoms in length and is derived from palmitic acid. In some of these embodiments, the thioalkoxy has an alkyl group that is of lauric acid, myristic acid, palmitic acid, stearic acid, oleic acid or linoleic acid. In some of these embodiments, each of F₁ and F₂ is independently a thioalkoxy as described herein, and can be the same or different, preferably the same. In some of these embodiments, F₃ and F₄ are each hydrogen.

According to some of any of the embodiments described herein, M is carboxy and at least one, or at least two, of F₁, F₂, F₃ and F₄ is independently a carboxy. In some of these embodiments, the carboxy is of at least 10 carbon atoms in length, for example, of from 8 to 40, or of from 10 to 40, or of from 10 to 30, carbon atoms in length. In exemplary embodiments, the carboxy is 16 carbon atoms in length and is derived from palmitic acid. In some of these embodiments, the carboxy has an alkyl group that is of lauric acid, myristic acid, palmitic acid, stearic acid, oleic acid or linoleic acid. In some of these embodiments, at least one or both of F₁ and F₂ is a carboxy as described herein in any of the respective embodiments. In some of these embodiments, both of F₁ and F₂ is a carboxy as described herein in any of the respective embodiments, which can be the same or different and is preferably the same. In some of any of these embodiments, at least one of F₃ and F₄ is an alkyl, which can be the same or different. In some such embodiments, the alkyl is a short alkyl of 1 to 6, or 1 to 4 carbon atoms in length, for example, methyl. Alternatively, each of F₃ and F₄ is hydrogen.

According to some of any of the embodiments described herein, J is absent.

According to some of any of the embodiments described herein, J is absent and M is other than amido.

According to some of any of the embodiments described herein, J is absent and M is carboxy.

According to some of any of the embodiments described herein, J is absent, and at least one, or at least two, of F₁, F₂, F₃ and F₄ is independently a thioalkoxy. In some of these embodiments, the thioalkoxy is of at least 10 carbon atoms in length, for example, of from 8 to 40, or of from 10 to 40, or of from 10 to 30, carbon atoms in length. In exemplary embodiments, the alkyl is 15 carbon atoms in length and is derived from palmitic acid. In some of these embodiments, the thioalkoxy has an alkyl group that is of lauric acid, myristic acid, palmitic acid, stearic acid, oleic acid or linoleic acid. In some of these embodiments, each of F₁ and F₂ is independently a thioalkoxy as described herein, and can be the same or different, preferably the same. In some of these embodiments, F₃ and F₄ are each hydrogen.

According to some of any of the embodiments described herein, J is absent, M is carboxy, and at least one, or at least two, of F₁, F₂, F₃ and F₄ is independently a thioalkoxy. In some of these embodiments, the thioalkoxy is of at least 10 carbon atoms in length, for example, of from 8 to 40, or of from 10 to 40, or of from 10 to 30, carbon atoms in length. In exemplary embodiments, the alkyl is 15 carbon atoms in length and is derived from palmitic acid. In some of these embodiments, the thioalkoxy has an alkyl group that is of lauric acid, myristic acid, palmitic acid, stearic acid, oleic acid or linoleic acid. In some of these embodiments, each of F₁ and F₂ is independently a thioalkoxy as described herein, and can be the same or different, preferably the same. In some of these embodiments, F₃ and F₄ are each hydrogen.

According to some of any of the embodiments described herein, J is absent, and at least one, or at least two, of F₁, F₂, F₃ and F₄ is independently a carboxy. In some of these embodiments, the carboxy is of at least 10 carbon atoms in length, for example, of from 8 to 40, or of from 10 to 40, or of from 10 to 30, carbon atoms in length. In exemplary embodiments, the carboxy is 16 carbon atoms in length and is derived from palmitic acid. In some of these embodiments, the carboxy has an alkyl group that is of lauric acid, myristic acid, palmitic acid, stearic acid, oleic acid or linoleic acid. In some of these embodiments, at least one or both of F₁ and F₂ is a carboxy as described herein in any of the respective embodiments. In some of these embodiments, both of F₁ and F₂ is a carboxy as described herein in any of the respective embodiments, which can be the same or different and is preferably the same. In some of any of these embodiments, at least one of F₃ and F₄ is an alkyl, which can be the same or different. In some such embodiments, the alkyl is a short alkyl of 1 to 6, or 1 to 4 carbon atoms in length, for example, methyl. Alternatively, each of F₃ and F₄ is hydrogen.

According to some of any of the embodiments described herein, J is absent, M is carboxy, and at least one, or at least two, of F₁, F₂, F₃ and F₄ is independently a carboxy. In some of these embodiments, the carboxy is of at least 10 carbon atoms in length, for example, of from 8 to 40, or of from 10 to 40, or of from 10 to 30, carbon atoms in length. In exemplary embodiments, the carboxy is 16 carbon atoms in length and is derived from palmitic acid. In some of these embodiments, the carboxy has an alkyl group that is of lauric acid, myristic acid, palmitic acid, stearic acid, oleic acid or linoleic acid. In some of these embodiments, at least one or both of F₁ and F₂ is a carboxy as described herein in any of the respective embodiments. In some of these embodiments, both of F₁ and F₂ is a carboxy as described herein in any of the respective embodiments, which can be the same or different and is preferably the same. In some of any of these embodiments, at least one of F₃ and F₄ is an alkyl, which can be the same or different. In some such embodiments, the alkyl is a short alkyl of 1 to 6, or 1 to 4 carbon atoms in length, for example, methyl. Alternatively, each of F₃ and F₄ is hydrogen.

According to some of any of the embodiments described herein for Formula IV when J is absent, Q is —C(CH₃)₂—.

According to some of any of the embodiments described herein for Formula IV when J is absent and M is carboxy, Q is —C(CH₃)₂—.

Herein, the length of the hydrocarbon represented by the variable K refers to the number of atoms separating J and M (i.e., along the shortest path between J and M) as depicted in Formula IV, in cases when J is not absent, or the number of atoms separating M and the lipid skeleton formed of F₁, F₂, F₃ and F₄.

When K is a substituted hydrocarbon, M may be attached to a carbon atom of the hydrocarbon per se, or be attached to a substituent of the hydrocarbon.

In some embodiments, K is an all-carbon hydrocarbon.

In some embodiments, K is an unsubstituted hydrocarbon.

In some embodiments, K is an unsubstituted all-carbon hydrocarbon.

In some of any of these embodiments, K is an alkyl (an alkylene chain or linking group), preferably unsubstituted, and optionally being a short alkyl or alkylene of 1 to 6, or 1 to 4, or 1 to 2, carbon atoms in length.

According to some of any of the embodiments described herein, K is absent.

According to some of any of the embodiments described herein, J is absent, as described herein in any of the respective embodiments, and K is absent. In some of these embodiments, M is carboxy.

According to some of any of the embodiments described herein, Q is a hydrocarbon substituted by at least one aryl (e.g., phenyl).

In some of these embodiments, Q is a hydrocarbon which is an all-carbon hydrocarbon, and is some of these embodiments the hydrocarbon is alkyl (an alkylene linking group), preferably a short alkyl (or alkylene) of from 1 to 6, or from 1 to 4, preferably 1 or 2, carbon atoms in length, substituted by at least one aryl (e.g., phenyl).

According to some of any of the embodiments described herein, Q is a methylene substituted by at least one aryl (e.g., phenyl).

According to some of any of the embodiments described herein, J is —P(═O)(OH)—O—; M is amido; and Q is a hydrocarbon substituted by at least one aryl (e.g., phenyl), as described herein in any of the respective embodiments and any combination thereof.

According to some of any of the embodiments described herein, J is —P(═O)(OH)—O—; M is amido; and Q is a methylene substituted by at least one aryl (e.g., phenyl).

In some embodiments of any of the embodiments described herein for Formula I, the lipid moiety represented by the variable X has the general Formula III:

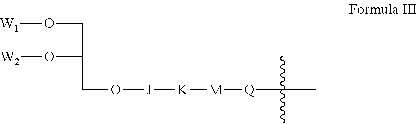

wherein:
the dashed (curved) line denotes an attachment point to the respective Y backbone unit;
W₁ and W₂ are each independently hydrogen, alkyl, alkenyl, alkynyl or acyl, wherein at least one of W₁ and W₂ is not hydrogen;
J is —P(═O)(OH)—O—, or J is absent (such that K is attached directly to the depicted oxygen atom of a glycerol moiety);
K is a substituted or unsubstituted hydrocarbon from 1 to 10 carbon atoms in length;
M is a linking group which is —O—, —S—, amino, sulfinyl, sulfonyl, phosphate, phosphonyl, phosphinyl, carbonyl, thiocarbonyl, urea, thiourea, carbamyl, thiocarbamyl, amido, carboxy, or sulfonamide, or M is absent (such that K is attached directly to Q); and
Q is a substituted or unsubstituted hydrocarbon from 1 to 10 carbon atoms in length, or Q is absent.

Q is attached to a backbone unit of the polymeric backbone according to any of the respective embodiments described herein, or alternatively, when Q is absent, M is attached to the aforementioned backbone unit.

When M is absent, Q is also absent, and K is attached to a backbone unit of the polymeric backbone according to any of the respective embodiments described herein.

In some embodiments of any of the embodiments described herein for Formula III, one of W₁ and W₂ is hydrogen and the other is not hydrogen.

In some embodiments of any of the embodiments described herein for Formula III, neither W₁ nor W₂ is hydrogen.

In some embodiments of any of the embodiments described herein for Formula III, at least one of W₁ and W₂ is an alkyl, alkenyl, alkynyl or acyl, which is from 10 to 30 carbon atoms in length. In some embodiments, each of W₁ and W₂ is from 10 to 30 carbon atoms in length.

Examples of acyl groups which may optionally serve independently as W₁ and/or W₂ include, without limitation, lauroyl, myristoyl, palmitoyl, stearoyl, palmitoleoyl, oleoyl, and linoleoyl.

In some embodiments of any of the embodiments described herein for Formula III, J is —P(═O)(OH)—O— (e.g., the lipid moiety is a glycerophospholipid).

Herein, the length of the hydrocarbon represented by the variable K refers to the number of atoms separating J and M (i.e., along the shortest path between J and M) as depicted in Formula III.

When K is a substituted hydrocarbon, M may be attached to a carbon atom of the hydrocarbon per se, or be attached to a substituent of the hydrocarbon.

In some embodiments of any of the embodiments described herein for Formula III, K is an acyl moiety (e.g., —C(═O)—C(CH₃)₂—). In some such embodiments, J is absent, such that K is attached directly to the depicted oxygen atom of a glycerol moiety. In some such embodiments, K comprises a carbonyl linking group (—C(═O)—), which attaches to the oxygen atom of a glycerol moiety via an ester bond.

In some embodiments of any of the embodiments described herein for Formula III, K is an ethanolamine moiety (e.g., —CH₂—CH₂—NH—, or —CH₂—CH₂— attached to a nitrogen atom), a serine moiety (e.g., —CH₂—CH(CO₂H)—NH—, or —CH₂—CH(CO₂H)— attached to a nitrogen atom), a glycerol moiety (e.g., —CH(OH)—CH(OH)—CH—O—) and an inositol moiety (e.g., -cyclohexyl (OH)₄—O—). In some embodiments, J is —P(═O)(OH)—O—.

In some embodiments of any of the embodiments described herein for Formula III, M is amido, optionally —C(═O)NH—.

In some embodiments, the nitrogen atom of the amido is attached to K. In some such embodiments, K is an ethanolamine or serine moiety described herein.

In some embodiments of any of the embodiments described herein for Formula III, Q is a substituted alkylene (e.g., of 1 to 6 or 1 to 4 or 1 to 2 carbon atoms in length, for example, a methylene group). In some such embodiments, M is amido or carboxy. In some embodiments, the C(═O) of the amido or the carboxy is attached to Q.

In some embodiments of any of the embodiments described herein for Formula III, Q is an alkylene group as described herein, and the methylene group substituted by one or two substituents, and at least one of these substituents is or comprises an aryl (e.g., phenyl). In some such embodiments, M is amido. In some embodiments, the C(═O) of the amido is attached to Q. Alternatively, M is carboxy and the C(═O) of the carboxy is attached to Q.

In some embodiments of any of the embodiments described herein for Formula III, Q is a methylene group substituted by two substituents, at least one being or comprising an aryl (e.g. phenyl), and the other can be, for example, an aryl (e.g., phenyl) or alkyl (e.g., of 1 to 6 or 1 to 4, carbon atoms in length). In some embodiments, the methylene group is substituted by an alkyl groups (e.g., C₁₋₄-alkyl) and an aryl (e.g., phenyl). In some such embodiments, M is amido. In some such embodiments, M is carboxy.

In some embodiments of any of the embodiments described herein for Formula III, Q is a substituted alkylene (e.g., of 1 to 6 or 1 to 4 or 1 to 2 carbon atoms in length, for example, a methylene group). In some such embodiments, M is amido or carboxy. In some embodiments, the C(═O) of the amido or the carboxy is attached to Q.

In some embodiments of any of the embodiments described herein for Formula III, Q is an alkylene group as described herein, and the methylene group substituted by one or two substituents. In some embodiments, the methylene group is substituted by one or two alkyl groups (e.g., C₁₋₄-alkyl). In some such embodiments, M is other than amido. In some such embodiments, M is carboxy.

In some embodiments of any of the embodiments described herein for Formula III, Q is a methylene group substituted by two substituents. In some embodiments, the methylene group is substituted by two alkyl groups (e.g., C₁₋₄-alkyl). In some embodiments, the alkyl groups are methyl, such that Q is dimethylmethylene (—C(CH₃)₂—). In some such embodiments, M is other than amido. In some such embodiments, M is carboxy.

According to some of any of the embodiments described herein for Formula III, when M is amido, Q is an alkylene that is substituted by at least one aryl as described herein in any of the respective embodiments.

According to some of any of the embodiments described herein, M is other than amido, and Q is as described herein in any of the respective embodiments.

In some embodiments of any of the embodiments described herein for Formula III, M and Q are each absent, and K is terminated by a substituted or unsubstituted methylene group, according to any of the respective embodiments described herein with respect to Q, for example, a methylene group substituted by two substituents (e.g., dimethylmethylene (—C(CH₃)₂—)). In some embodiments, K further comprises a carbonyl group according to any of the respective embodiments described herein.

In some embodiments of any of the embodiments described herein, J, M and Q are each absent. In some such embodiments, K comprises a carbonyl linking group (—C(═O)—) attached directly to the depicted oxygen atom of a glycerol moiety (via an ester bond), and further comprises a substituted or unsubstituted methylene group (e.g., dimethylmethylene). In some embodiment, K consists of a carbonyl linking group attached directly to the depicted oxygen atom of a glycerol moiety (via an ester bond), and a substituted or unsubstituted methylene group, for example, K is —C(═O)—C(CH₃)₂—.

According to some of any of the embodiments described herein for Formula IV, F₁ is as described herein for OW₁. According to some of these embodiments, F₃ and F₄ are each hydrogen. According to some of these embodiments, J is absent. According to some of these embodiments, M is other than amido.

According to some of any of the embodiments described herein for Formula IV, F₂ is as described herein for OW₂. According to some of these embodiments, F₃ and F₄ are each hydrogen. According to some of these embodiments, J is absent. According to some of these embodiments, M is other than amido.

According to some of any of the embodiments described herein for Formula IV, F₁ is as described herein for OW₁ and F₂ is as described herein for OW₂. According to some of these embodiments, F₃ and F₄ are each hydrogen. According to some of these embodiments, J is absent. According to some of these embodiments, M is other than amido.

According to some of any of the embodiments described herein, the lipid moiety does not include a moiety of Formula III as described herein.

According to some of any of the embodiments described herein, the lipid moiety has a moiety of Formula III as described herein, provided that M is other than amido (e.g., M is carboxy) and/or that Q comprises an aryl substituent, as described herein.

According to some of any of the embodiments described herein for Formula IV, excluded from the scope of the present embodiments are polymeric compounds in which the lipid moiety X is as described in WO 2017/109784.

### Targeting Moiety:

In some embodiments of any of the embodiments described herein, at least a portion of the monomeric units of the polymeric moiety comprise a targeting moiety (according to any of the embodiments described herein relating to a targeting moiety).

Herein, a “targeting moiety” refers to a moiety which is capable of bringing a compound (e.g., a compound according to some embodiments of the invention) into proximity with a selected substance and/or material (which is referred to herein as a “target”). The target is optionally a cell (e.g., a proliferating cell associated with the proliferative disease or disorder), wherein the proximity is such that the targeting moiety facilitates attachment and/or internalization of the compound into a target cell, and such that the compound may exert a therapeutic effect.

In some of any of the embodiments described herein, a targeting moiety comprises a backbone unit Y according to any of the respective embodiments described herein, and optionally a linking moiety L according to any of the respective embodiments described herein and/or moiety Z according to any of the respective embodiments described herein, for example, wherein a substituent according to any of the respective embodiments described herein comprises (and optionally consists of) the targeting moiety.

For example, in some embodiments wherein at least a portion of backbone units Y have the formula —CR₄R₅—CR₆D- (as described herein in any of the respective embodiments), any one or more of R₄-R₆ and D (optionally wherein D is R₇ as described herein) comprises a targeting moiety according to any of the respective embodiments described herein (e.g., wherein any one or more of R₄-R₆ and D is a substituted group, comprising a substituent which is a targeting moiety), and optionally any one or more R₄-R₆ and D is a targeting moiety. However, many other structures of monomeric units comprising a substituent which comprises (and optionally consist of) a targeting moiety are also encompassed by embodiments of the invention.

In some embodiments, the polymeric moiety is a copolymer moiety as described herein in any of the respective embodiments, wherein at least one monomeric unit as described herein comprises a targeting moiety (according to any of the respective embodiments described herein) and at least one other monomeric unit does not comprise such a targeting moiety. The distribution of a monomeric unit comprising a targeting moiety may be in accordance with any distribution described herein of a monomeric unit in a copolymer moiety (e.g., random, alternating, periodic copolymer, and/or block copolymer). In any of the embodiments described herein wherein m is at least 1, at least a portion of the monomeric units comprising a targeting moiety according to any of the respective embodiments described herein. In some such embodiments, the at least a portion of the monomeric units which comprising a targeting moiety according to any of the respective embodiments described herein, are monomeric units which do not comprise a pendant group represented by (-L-Z) as described herein in any of the respective embodiments. In some such embodiments, the number of the monomeric units comprising the targeting moiety according to any of the respective embodiments is represented by the variable m according to any of the respective embodiments described herein. In some such embodiments, none of the monomeric units comprising the pendant group represented by (-L-Z) comprise the aforementioned targeting moiety.

In any of the embodiments described herein wherein m is at least 1, each of the monomeric units which do not comprise the pendant group represented by (-L-Z)(the number of which is represented by the variable m) comprises a targeting moiety (according to any of the respective embodiments described herein). In some such embodiments, each of the monomeric units comprising a targeting moiety (according to any of the respective embodiments described herein) is a monomeric unit which does not comprise the pendant group represented by (-L-Z), that is, none of the monomeric units comprising the pendant group represented by (-L-Z) comprise the aforementioned targeting moiety, and each of the monomeric units which does not comprise the pendant group represented by (-L-Z) comprises the aforementioned targeting moiety.

In any of the embodiments described herein wherein m is at least 1, a monomeric unit comprising a targeting moiety may consist essentially of a backbone unit Y(according to any of the respective embodiments described herein) substituted by one or more targeting moieties (according to any of the respective embodiments described herein).

In some of any of the embodiments described herein, the at least a portion of the monomeric units which comprising a targeting moiety according to any of the respective embodiments described herein, are monomeric units which comprise a pendant group represented by (-L-Z) as described herein in any of the respective embodiments. In some such embodiments, the number of the monomeric units comprising the targeting moiety according to any of the respective embodiments is represented by the variable n according to any of the respective embodiments described herein (i.e., each of the monomeric units comprising a targeting moiety according to any of the respective embodiments described herein is a monomeric unit which comprises the pendant group). In some such embodiments, none of the monomeric units which do not comprise the pendant group represented by (-L-Z) comprise the aforementioned targeting moiety.

In some of any of the embodiments described herein, monomeric unit comprising a targeting moiety may optionally be different (optionally considerably different) in structure (i.e., in the structure of Y and/or L and/or Z, if present, as defined in any of the embodiments described herein) than another monomeric unit comprising a targeting moiety. For example, the backbone unit Y of a monomeric unit comprising a targeting moiety may optionally be different in structure than a backbone unit Y of other monomeric units in the polymeric moiety (according to any of the respective embodiments described herein).

In any of the embodiments described herein wherein m is at least 1, the polymeric moiety comprises a monomeric unit which comprises a targeting moiety, and the monomeric unit is at a terminus of the polymeric moiety distal to the lipid moiety. In such embodiments, the compound represented by general Formula I has the Formula Ib:

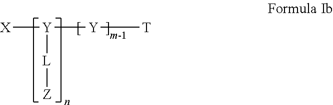

wherein:
T is a monomeric unit comprising a targeting moiety (according to any of the respective embodiments described herein);
X and T are attached to distal termini of the moiety represented by [Y(-L-Z)]n[Y]m−1; and
X, Y, L, Z, n and m are defined in accordance with any of the embodiments described herein relating to Formula I, with the proviso that m is at least 1.

It is to be understood that T in Formula Ib is a type of monomeric unit represented by Y (i.e., without the pendant group represented by (-L-Z)) in formulas I and Ia, and the number of monomeric units represented by Y(i.e., without the pendant group represented by (-L-Z)) other than T is represented by the value m−1, such that the total number of monomeric units without the pendant group represented by (-L-Z)), including T, is represented by the variable m, as in formulas I and Ia.

In some embodiments, m is 1, such that m−1 is zero, and the compound represented by Formula Ib consequently has the formula: X—[Y(-L-Z)]n-T, wherein L, T, X, Y, Z and n are defined in accordance with any of the embodiments described herein.

A monomeric unit comprising a targeting moiety according to any of the respective embodiments described herein may optionally be prepared by preparing a monomer comprising a targeting moiety, and using the monomer to prepare a polymeric moiety described herein (e.g., by polymerization of monomers according to any of the respective embodiments described herein) and/or by modifying a monomeric unit in a polymeric moiety subsequently to preparation of a polymeric moiety (e.g., by polymerization of monomers according to any of the respective embodiments described herein), using any suitable technique known in the art, including, but not limited to, techniques for conjugation.

In some embodiments of any of the embodiments described herein relating to a targeting moiety, the targeting moiety does not comprise a moiety having general Formula II (according to any of the respective embodiments described herein). For example, even if a moiety represented by Formula II is capable of forming a bond with a target as described herein, the phrase “targeting moiety”, in some embodiments, is to be understood as relating to a moiety distinct from a moiety represented by variable Z (having general Formula II).

In some embodiments of any one of the embodiments described herein, the pendant group represented by (-L-Z) is selected so as not to form a bond with the target and/or so as not to include a structure and/or property of a targeting moiety as described herein in any one of the respective embodiments. For example, in embodiments wherein a targeting moiety comprising a nucleophilic group (according to any of the respective embodiments described herein)—for example, an amine group—is capable of forming a bond (e.g., covalent bond) with a target, the variable Z (having general Formula II) is optionally selected such that the depicted amine/ammonium group is a tertiary amine/ammonium (i.e., no more than one of R₁-R₃ is hydrogen) or quaternary ammonium (i.e., none of R₁-R₃ is hydrogen), preferably a quaternary ammonium (e.g., comprising a trimethylamino group, such as in phosphocholine). Tertiary amine groups, and especially quaternary ammonium groups, may be significantly less reactive nucleophilic groups than primary and secondary amine groups.

In some embodiments of any of the embodiments described herein relating to a targeting moiety, the targeting moiety comprises (and optionally consists of) at least one functional group capable of forming a covalent bond or non-covalent bond (preferably a selective non-covalent bond) with a substance and/or material (which is referred to herein as a “target”), e.g., at a surface of the target (e.g., a surface of a cell and/or tissue).

Herein, the phrase “functional group” encompasses chemical groups and moieties of any size and any functionality described herein (for example, any functionality capable of forming a covalent bond or non-covalent bond with a target).

A non-covalent bond according to any of the respective embodiments described herein may optionally be effected by non-covalent interactions such as, without limitation, electrostatic attraction, hydrophobic bonds, hydrogen bonds, and aromatic interactions.

In some embodiments, the targeting moiety comprises a functional group capable of forming a non-covalent bond which is selective for the target, e.g., an affinity (e.g., as determined based on a dissociation constant) of the targeting moiety and/or functional group to the target is greater than an affinity of the of the targeting moiety and/or functional group to most (or all) other compounds capable of forming a non-covalent bond with the targeting moiety.

In some embodiments of any one of the embodiments described herein, the functional group(s) are capable of forming a covalent bond with one or more specific functional groups (e.g., hydroxy, amine, thiohydroxy and/or oxo groups) which are present on the target (e.g., a target according to any of the respective embodiments described herein).

Examples of functional groups (in a targeting moiety) capable of forming a covalent bond with a target (according to any of the respective embodiments described herein) and the type of covalent bonds they are capable of forming, include, without limitation:
nucleophilic groups such as thiohydroxy, amine (e.g., primary or secondary amine) and hydroxy, which may form covalent bonds with, e.g., a nucleophilic leaving group (e.g., any nucleophilic group described herein), Michael acceptor (e.g., any Michael acceptor described herein), acyl halide, isocyanate and/or isothiocyanate (e.g., as described herein) in a target;
nucleophilic leaving groups such as halo, azide (—N₃), sulfate, phosphate, sulfonyl (e.g. mesyl, tosyl), N-hydroxysuccinimide (NHS)(e.g. NHS esters), sulfo-N-hydroxysuccinimide, and anhydride, which may form covalent bonds with, e.g., a nucleophilic group (e.g., as described herein) in a target;
Michael acceptors such as enones (e.g., maleimide, acrylate, methacrylate, acrylamide, methacrylamide), nitro groups and vinyl sulfone, which may form covalent bonds with, e.g., a nucleophilic group (e.g., as described herein) in a target, optionally thiohydroxy;
dihydroxyphenyl groups (according to any of the respective embodiments described herein), which may form covalent bonds with, e.g., a nucleophilic group (e.g., as described herein) and/or a substituted or unsubstituted phenyl group (e.g., another dihydroxyphenyl group) in a target, as described herein;
an acyl halide (—C(═O)-halogen), isocyanate (—NCO) and isothiocyanate (—N═C═S) group, which may form covalent bonds with, e.g., a nucleophilic group (e.g., as described herein) in a target;
a carboxylate (—C(═O) OH) group, which may form a covalent bond with, e.g., a hydroxyl group in a target to form an ester bond and/or an amine group (e.g., primary amine) in a target to form an amide bond (optionally by reaction with a coupling reagent such as a carbodiimide); and/or a carboxylate group is in a target and may form an amide or ester bond with an amine or hydroxyl group, respectively, in the targeting moiety;
an oxo group (optionally in an aldehyde group (—C(═O) H)), which may form a covalent imine bond with an amine group (e.g., a primary amine) in a target; and/or an oxo group (optionally in an aldehyde group) is in a target and may form a covalent imine bond with an amine groups in the targeting moiety; and/or
thiohydroxy groups, which may form covalent disulfide (—S—S—) bonds with a thiohydroxy group in a target.

Modification of a monomer (e.g., prior to polymerization) or a monomeric unit of a polymeric moiety (e.g., subsequent to polymerization) to comprise any of the functional groups described herein may optionally be performed using any suitable technique for conjugation known in the art. The skilled person will be readily capable of selecting a suitable technique for any given molecule to be modified.

Herein, the term “dihydroxyphenyl” refers to an aryl group (as defined herein) which is a phenyl substituted by two hydroxyl groups at any positions thereof. The phenyl may optionally be substituted by additional substituents (which may optionally comprise additional hydroxyl groups), to thereby form a substituted dihydroxyphenyl group; or alternatively, the phenyl comprises no substituents other than the two hydroxyl groups, such that the dihydroxyphenyl group is an unsubstituted dihydroxyphenyl group.

In some embodiments of any one of the embodiments described herein, the dihydroxyphenyl group is an ortho-dihydroxyphenyl (wherein the hydroxyl groups are attached to the phenyl at adjacent positions) or a para-dihydroxyphenyl (wherein the hydroxyl groups are attached to opposite sides of the phenyl ring), each being a substituted or unsubstituted dihydroxyphenyl. In some such embodiments, the ortho-dihydroxyphenyl or para-dihydroxyphenyl is an unsubstituted dihydroxyphenyl.

A dihydroxyphenyl group according to any of the respective embodiments described herein may optionally bond covalently and/or non-covalently to a target according to any one or more attachment mechanism described for dihydroxyphenyl (catechol) groups in Lec et al. [PNAS 2006, 103:12999-13003], Brodie et al. [Biomedical Materials 2011, 6:015014] and/or International Patent Application PCT/IL2015/050606 (published as WO 2015/193887).

In some embodiments of any one of the embodiments described herein, the functional group capable of forming a bond to a target is a functional group capable of forming a covalent bond with an amine group, optionally a primary amine group. In some such embodiments, the target comprises on or more amino acids or amino acid residues, for example, a peptide or polypeptide of any length (e.g., at least two amino acid residues, for example, proteins), and the amine groups may optionally be lysine side chain amine groups and/or N-terminal amine groups. In some embodiments, the target comprises an extracellular matrix protein, for example, collagen. In some embodiments, the target comprises cartilage (e.g., articular cartilage).

In some embodiments of any one of the embodiments described herein, the targeting moiety comprises (and optionally consists of) at least one functional group capable of forming a non-covalent bond with the target (e.g., as described herein in any one of the respective embodiments).

In some embodiments of any one of the embodiments described herein, a functional group capable of forming a non-covalent bond with the target comprises (and optionally consists of) a polysaccharide and/or polypeptide (e.g., a protein and/or fragment thereof), wherein the target optionally comprises a ligand of the polysaccharide and/or polypeptide; and/or the target comprises a polysaccharide and/or polypeptide (e.g., a protein and/or fragment thereof) and the functional group capable of forming a non-covalent bond with the target is a ligand of the polysaccharide and/or polypeptide.

Examples of suitable polysaccharides and/or polypeptides, and ligands thereof, include, without limitation:
avidin or streptavidin as a polypeptide described herein, and biotin as a ligand thereof;
a polysaccharide-binding polypeptide as a polypeptide described therein, and a complementary polysaccharide as a ligand thereof (or a complementary polysaccharide-binding polypeptide as a ligand of a polysaccharide described herein);
a collagen-binding polypeptide as a polypeptide described therein, and a complementary collagen as a ligand thereof (or a collagen as a polypeptide described herein and a complementary collagen-binding polypeptide as a ligand thereof);
a cell receptor expressed by a cell, and a ligand selectively bound by the receptor;
an antibody towards any antigen (e.g., wherein the target described herein optionally comprises the antigen) or a fragment of such an antibody as a polypeptide described herein, and the respective antigen as a ligand thereof; and
an antibody mimetic towards any antigen (e.g., wherein the target described herein optionally comprises the antigen).

Examples of cell receptors expressed by a cell include, without limitation, receptors characteristic of a particular type of cell and/or tissue, and receptors overexpressed by a cancer cell. The cell receptor or the cell is optionally a target described herein, and the targeting moiety optionally comprises any ligand of the receptor. Examples of such ligands include, without limitation, transferrin, a ligand of transferrin receptor which may optionally target transferrin receptor overexpressed by some cancer cells; keratinocyte growth factor (KGF or FGF7) which is specific for cells of epithelial origin, and may optionally target KGF receptor such as that overexpressed by an endometrial carcinoma or pancreatic carcinoma [Visco et al., Int J Oncol 1999, 15:431-435; Siegfried et al., Cancer 1997, 79:1166-1171]; and epidermal growth factor (EGF) which may optionally target an EGF receptor, optionally an erbB, such as that overexpressed by gliomas and endometrial carcinomas [Normanno et al., Curr Drug Targets 2005, 6:243-257]). As used herein, the term “antibody” encompasses any type of immunoglobin.

As used herein, the phrase “antibody mimetic” encompasses any type of molecule, optionally a polypeptide, referred as such in the art capable of selectively binding an antigen (e.g., non-covalently). Non-limiting examples of antibody mimetics include affibodies, affilins, affimers, affitins, alphabodies, anticalins, avimers, DARPins, Fynomers, Kunitz domain peptides, and monobodies, e.g., as described in Nygren [FEBS J 2008, 275:2668-2676], Ebersbach et al. [J Mol Biol 2007, 372:172-185], Johnson et al. [Anal Chem 2012, 84:6553-6560], Krehenbrink et al. [J Mol Biol 2008, 383:1058-1068], Desmet et al. [Nature Comm 2014, 5:5237], Skerra [FEBS J 2008, 275:2677-2683], Silverman et al. [Nature Biotechnol 2005, 23:1556-1561], Stumpp et al. [Drug Discov Today 2008, 13:695-701], Grabulovski et al. [J Biol Chem 2007, 282:3196-3204], Nixon & Wood [Curr Opin Drug Discov Devel 2006, 9:261-268], Koide & Koide [Methods Mol Biol 2007, 325:95-109], and Gebauer & Skerra [Curr Opin Chem Biol 2009, 13:245-255], the contents of each of which are incorporated in their entirety, and especially contents regarding particular types of antibody mimetics.

As used herein, the phrase “polysaccharide-binding polypeptide” encompasses any polypeptide or oligopeptide (peptide chains of at least 2, and preferably at least 4 amino acid residues in length) capable of selectively binding (e.g., non-covalently) to a polysaccharide. A wide variety of polysaccharide-binding polypeptides and their binding specificities will be known to the skilled person, and include short peptide sequences (e.g., from 4 to 50, optionally 4 to 20 amino acid residues in length), and longer polypeptides such as proteins or fragments (e.g., carbohydrate-binding modules and/or domains) thereof. In addition, the phrase “polysaccharide-binding polypeptide” encompasses antibodies capable of specifically binding to a polysaccharide. Such antibodies will be available to the skilled person and/or the skilled person will know how to prepare such antibodies, using immunological techniques known in the art.

Examples of polysaccharide-binding polypeptides which may be used in some of any one of the embodiments of the invention include, without limitation, carbohydrate-binding modules (CBMs); and hyaluronic acid-binding peptides, polypeptides and/or modules (e.g., having a sequence as described in any of International Patent Application publication WO 2013/110056; International Patent Application publication WO 2014/071132; Barta et al. [Biochem J 1993, 292:947-949], Kohda et al. [Cell 1996, 86:767-775], Brisset & Perkins [FEBS Lett 1996, 388:211-216], Peach et al. [J Cell Biol 1993, 122:257-264], Singh et al. [Nature Materials 2014, 13:988-995], and Zaleski et al. [Antimicrob Agents Chemother 2006, 50:3856-3860], the contents of each of which are incorporated in their entirety, and especially contents regarding particular polysaccharide-binding polypeptides), for example, GAHWQFNALTVR (a hyaluronic acid-binding peptide sequence).

Examples of CBMs which may be used in some of any one of the embodiments of the invention, include, without limitation, CBMs belonging to the families CBM3, CBM4, CBM9, CBM10, CBM17 and/or CBM28 (which may optionally be used to bind cellulose, e.g., in a cellulose-containing target); CBM5, CBM12, CBM14, CBM18, CBM19 and/or CBM33 (which may optionally be used to bind chitin and/or other polysaccharides comprising N-acetylglucosamine, e.g., in a chitin-containing target); CBM15 (which may optionally be used to bind hemicellulose, e.g., in a hemicellulose-containing target); and/or CBM20, CBM21 and/or CBM48 (which may optionally be used to bind starch and/or glycogen, e.g., in a starch-containing and/or glycogen-containing target).

As used herein, the phrase “collagen-binding polypeptide” encompasses any polypeptide or oligopeptide (peptide chains of at least 2, and preferably at least 4 amino acid residues in length) capable of selectively binding (e.g., non-covalently) to a collagen (e.g., one type of collagen, some types of collagen, all types of collagen), including glycosylated polypeptides and oligopeptides such as peptidoglycans and proteoglycans. A wide variety of collagen-binding polypeptides and their binding specificities will be known to the skilled person, and include short peptide sequences (e.g., from 4 to 50, optionally 4 to 20 amino acid residues in length), and longer polypeptides such as proteins or fragments (e.g., collagen-binding domains) thereof. In addition, the phrase “collagen-binding polypeptide” encompasses antibodies capable of specifically binding to a collagen. Such antibodies will be available to the skilled person and/or the skilled person will know how to prepare such antibodies, using immunological techniques known in the art.

Examples of collagen-binding polypeptides which may be used in embodiments of the invention include, without limitation, collagen-binding proteins (e.g., decorin), fragments thereof and/or other polypeptides as described in U.S. Pat. No. 8,440,618, Abd-Elgaliel & Tung [Biopolymers 2013, 100:167-173], Paderi et al. [Tissue Eng Part A 2009, 15:2991-2999], Rothenfluh et al. [Nat Mater 2008, 7:248-254] and Helms et al. [J Am Chem Soc 2009, 131:11683-11685] (the contents of each of which are incorporated in their entirety, and especially contents regarding particular collagen-binding polypeptides), for example, the sequence WYRGRL.

It is expected that during the life of a patent maturing from this application many relevant functional groups and moieties for binding will be developed and/or uncovered and the scope of the terms “targeting moiety”, “functional group”, “cell receptor”, “antibody”, “antibody mimetic”, “collagen-binding polypeptide” and “polysaccharide-binding polypeptide” and the like is intended to include all such new technologies a priori.

In some embodiments of any of the embodiments described herein, a functional group in a targeting moiety (according to any of the respective embodiments described herein) is attached to a linking group (as defined herein). The linking group may optionally be any linking group or linking moiety described herein, including, without limitation, a substituted or unsubstituted hydrocarbon. In some embodiments, the targeting moiety (optionally a substituent of a backbone unit Y) consists essentially of a functional group attached to the rest of the polymeric moiety via the linking group.

A functional group may optionally be attached to the linking moiety by a covalent bond obtainable by a reaction between two functional groups, for example, any covalent bond and/or functional groups described herein in the context of forming a covalent bond between a functional group and a target.

In some embodiments of any of the embodiments described herein relating to a functional group comprising a peptide or polypeptide, an amino acid residue of the peptide or polypeptide is optionally attached to a linking group of the targeting moiety, for example, via an amide bond formed from an amine or carboxylate group in the peptide or polypeptide (e.g., in an N-terminus, a lysine side chain, a C-terminus, a glutamate side chain and/or an aspartate side chain), an ester bond formed from a hydroxyl or carboxylate group in the peptide or polypeptide (e.g., in a serine side chain, a threonine side chain, a C-terminus, a glutamate side chain and/or an aspartate side chain), and/or a disulfide bond formed from a thiohydroxy group in the peptide or polypeptide (e.g., in a cysteine side chain). In some embodiments, an amino acid residue attached to the linking group is an N-terminal and/or C-terminal residue, for example, any amino acid residue attached via an N-terminal amino group or C-terminal carboxylate group, and/or a terminal lysine, glutamate, aspartate, serine, threonine and/or cysteine residue attached via a side chain thereof.

In some embodiments, an amino acid residue and/or peptide (e.g., from 2 to 20 amino acid residues in length) is added to the N-terminus and/or C-terminus of a peptide or polypeptide sequence of a functional group (according to any of the respective embodiments described herein), and links the aforementioned sequence to a linking group. Examples of such terminal amino acid residues and/or peptides include, without limitation, glycine residues and peptides with a terminal glycine residue, which may be used to attach a linking group to an N-terminus or C-terminus (according to any of the respective embodiments described herein); serine and threonine residues and peptides with a terminal serine or threonine residue, which may be used to attach a linking group to hydroxyl group in a serine or threonine side chain, optionally via an ester bond (according to any of the respective embodiments described herein); and cysteine residues and peptides with a terminal cysteine residue, which may be used to attach a linking group to a peptide via a disulfide bond (according to any of the respective embodiments described herein).

In some embodiments, attachment of a peptide or polypeptide to a linking group via a terminal amino acid residue minimizes interference (e.g., steric interference) with the functionality of the peptide or polypeptide following attachment to the linking group.

In some embodiments, attachment of a peptide or polypeptide to a linking group via a terminal glycine facilitates attachment by minimizing interference (e.g., steric interference) of an amino acid side chain (which glycine lacks) with attachment to the linking group.

In some embodiments of any of the embodiments described herein wherein at least a portion of the monomeric units comprising a targeting moiety, the monomeric units comprising a targeting moiety are, on average, closer to a terminus of the polymeric moiety distal to the lipid moiety, e.g., an average distance (as measured in atoms or backbone units along the backbone of the polymeric moiety) of monomeric units comprising a targeting moiety from the lipid moiety is greater than an average distance of the other monomeric units from the lipid moiety.

In some embodiments, at least a portion (and optionally all) of the monomeric units comprising a targeting moiety form a block (of one or more monomeric units) near (and optionally at) a terminus of the polymeric moiety distal to the lipid moiety. In some such embodiments, the copolymer moiety contains a single monomeric unit which comprises a targeting moiety, and the monomeric unit is at a terminus of the polymeric moiety distal to the lipid moiety.

Without being bound by any particular theory, it is assumed that a targeting moiety located distal to the lipid moiety may be more effective as a targeting moiety (e.g., more effective at binding to a target), for example, due to the targeting moiety being less sterically shielded (e.g., by a surface to which the lipid moiety is associated) and therefore more exposed to and thus better able to make contact with targets in an aqueous environment.

In alternative embodiments, the polymeric moiety does not comprise a targeting moiety as described herein in any of the respective embodiments.

### Lipid Layers and Liposomes:

According to an aspect of some embodiments of the invention, there is provided a lipid bilayer (referred to herein interchangeably as simply a “bilayer”) comprising a polymeric compound according to any of the respective embodiments described herein. In some such embodiments, the bilayer further comprises at least one bilayer-forming lipid in addition to the polymeric compound. In some of any of the embodiments described herein or in any combination thereof, the at least one bilayer-forming lipid comprises at least one zwitterionic bilayer-forming lipid, as described herein, and optionally further comprises at least one negatively-charged bilayer-forming lipid.

Herein, the term “bilayer-forming lipid” encompasses any compound in which a bilayer may form from a pure aqueous solution of the compound, the bilayer comprising two parallel layers of molecules of the compound (referred to as a “lipid”).

Typically, the bilayer comprises relatively polar moieties of the lipid at the two surfaces of the bilayer, which may optionally comprise an interface with the aqueous solution and/or an interface with a solid surface; and relatively hydrophobic moieties of the lipid at the interior of the bilayer, at an interface between the two layers of lipid molecules which form the bilayer.

In some embodiments, a bilayer-forming lipid is an amphiphilic lipid.

As used herein, the term “amphiphilic lipid” refers to compounds comprising at least one hydrophilic moiety and at least one lipophilic moiety. Examples of amphiphilic lipids include, without limitation, fatty acids (e.g., at least 6 carbon atoms in length) and derivatives thereof such as phospholipids and glycolipids; sterols (e.g., cholesterol) and steroid acids.

Herein, the term “phospholipid” refers to a compound comprising a substituted or non-substituted phosphate group and at least one alkyl chain (optionally at least two alkyl chains) which is optionally at least 5 carbon atoms in length, optionally at least 7 atoms in length and optionally at least 9 atoms in length. The at least one alkyl chain is optionally a part of an acyl group (e.g., a fatty acid moiety) or an alkyl group per se (e.g., a fatty alcohol moiety). In some embodiments, the phosphate group and one or two (optionally two) alkyl chains (e.g., acyl or alkyl) are attached to a glycerol moiety via the oxygen atoms of glycerol.

In the context of the present embodiments, the term “phospholipid” encompasses lipids having a (phosphorylated) glycerol backbone (e.g., monoacylglyceride and/or diacylglyceride phospholipids), referred to as glycerophospholipids.

In some embodiments of any one of the embodiments described herein, the phospholipid is a glycerophospholipid. In some embodiments, the glycerophospholipid is a diacylglyceride, comprising two fatty acyl groups and one phosphate group attached to a glycerol backbone.

Examples of bilayer-forming lipids include glycerophospholipids (e.g., a glycerophospholipid according to any of the respective embodiments described herein). It is to be appreciated that the polymeric compound described herein may optionally be a bilayer-forming lipid which can form a bilayer per se or in combination with one or more additional bilayer-forming lipids.

In some embodiments of any one of the embodiments described herein, the bilayer-forming lipid comprises at least one charged group (e.g., one or more negatively charged groups and/or one or more positively charged groups).

In some embodiments, the bilayer-forming lipid is zwitterionic; comprising both (e.g., an equal number of) negatively charged and positively charged groups (e.g., one of each).

In some embodiments of any of the embodiments described herein, a mol ratio of the bilayer-forming lipid (comprised in addition to the polymeric compound) and the polymeric compound (according to any of the respective embodiments described herein) in the liposome is in a range of from 5:1 to 5,000:1 (bilayer-forming lipid: polymeric compound), optionally in a range of from 10:1 to 2,500:1, optionally in a range of from 25:1 to 1,000:1, and optionally in a range of from 50:1 to 500:1, including any intermediate values and subranges therebetween.

Herein throughout, the terms “mol ratio” and “molar ratio” are used interchangeably, and describe the ratio between the mol % of the indicated components in the lipid bilayer or liposome.

In some embodiments of any of the embodiments described herein relating to a bilayer, a mol ratio of the bilayer-forming lipid (comprised in addition to the polymeric compound) and the polymeric compound in the bilayer is in a range of from 10:1 to 1,000:1 (bilayer-forming lipid: polymeric compound), optionally in a range of from 10:1 to 500:1, optionally in a range of from 10:1 to 100:1, and optionally in a range of from 10:1 to 50:1, including any intermediate values and subranges therebetween.

In some embodiments of any of the embodiments described herein relating to a bilayer, a mol ratio of the bilayer-forming lipid (comprised in addition to the polymeric compound) and the polymeric compound in the bilayer is in a range of from 10:1 to 100:1 (bilayer-forming lipid: polymeric compound), optionally in a range of from 10:1 to 50:1, optionally in a range of from 30:1 to 40:1, including any intermediate values and subranges therebetween.

In some embodiments of any of the embodiments described herein relating to a bilayer, a molar ratio of the bilayer-forming lipid (comprised in addition to the polymeric compound) and the polymeric compound in the bilayer is in a range of from 10:1 to 1,000:1 (bilayer-forming lipid: polymeric compound), optionally in a range of from 100:1 to 1,00:1, optionally in a range of from 101:1 to 500:1, and optionally in a range of from 100:1 to 200:1, including any intermediate values and subranges therebetween.

The bilayer according to some of the present embodiments may optionally be closed upon itself (e.g., such that the bilayer has no edges), thereby forming an inner volume separated by the bilayer from the surrounding environment, which is referred to herein and in the art as a “liposome”. Alternatively or additionally, the bilayer may be open-faced and/or with edges.

According to an aspect of some embodiments of the invention, there is provided a liposome comprising at least one lipid bilayer according to any of the respective embodiments described herein.

As used herein and in the art, the term “liposome” refers to an artificially prepared vesicle comprising a bilayer composed of molecules of an amphiphilic lipid. In an aqueous medium, the bilayer is typically configured such that hydrophilic moieties of the amphiphilic lipid are exposed to the medium at both surfaces of the bilayer, whereas lipophilic moieties of the lipid are located in the internal portion of the bilayer, and therefore less exposed to the medium. Examples of liposomes which may be used in any one of the embodiments described herein include, without limitation, small unilamellar vesicles (SUV), large unilamellar vesicles (LUV) and large multilamellar vesicles (MLV).

As described herein, the liposome according to embodiments comprises, inter alia, at least one bilayer-forming lipid.

It is to be appreciated that the polymeric compound comprised by a liposome (according to any of the respective embodiments described herein) may optionally be a bilayer-forming lipid which can form a bilayer per se or in combination with one or more additional bilayer-forming lipids.

A liposome may optionally comprise a single bilayer (e.g., a unilamellar vesicle) or a plurality of bilayers (e.g., a multilamellar vesicle)—wherein each bilayer optionally independently forms a closed vesicle-comprising, for example, concentric bilayer vesicles and/or a plurality of separate bilayer vesicles encompassed by the same bilayer vesicle.

As used herein, the term “unilamellar” refers to liposomes characterized by a single lipid bilayer, whereas the term “multilamellar” refers to liposomes characterized by a multiple lipid bilayers, for example, concentric bilayers.

As used herein, the phrase “small unilamellar vesicle” refers to unilamellar liposomes of less than 100 nm in diameter, whereas the phrase “large unilamellar vesicle” refers to unilamellar liposomes at least 100 nm in diameter.

As used herein, the phrase “small multilamellar vesicle” refers to multilamellar liposomes of less than 100 nm in diameter, whereas the phrase “large multilamellar vesicle”, MLV, refers to multilamellar liposomes at least 100 nm in diameter.

In some embodiments of any one of the embodiments described herein, the liposomes comprise multilamellar vesicles. In some embodiments, the liposomes are primarily (more than 50 weight percent) multilamellar vesicles, preferably large multilamellar vesicles (MLV).

In some embodiments of any one of the embodiments described herein, the liposomes comprise small unilamellar vesicles. In some embodiments, the liposomes are primarily (more than 50 weight percent) small unilamellar vesicles.

In some embodiments of any one of the embodiments described herein, the liposomes comprise large unilamellar vesicles. In some embodiments, the liposomes are primarily (more than 50 weight percent) large unilamellar vesicles.

A liposome according to any of the respective embodiments described herein may be approximately spherical in shape or may have any alternative shape, such as an elongated tube and/or a flattened (e.g., sheet-like) shape.

In some embodiments of any one of the embodiments described herein, a concentration of phospholipids in liposomes in a composition or formulation as described herein is in a range of from 0.5 mM to 500 mM. In some embodiments, the concentration is in a range of from 0.5 mM to 150 mM. In some embodiments, the concentration is in a range of from 0.5 mM to 50 mM. In some embodiments, the concentration is in a range of from 0.5 mM to 10 mM. In some embodiments, the concentration is in a range of from 0.5 mM to 5 mM. In some embodiments, the concentration is in a range of from 1 mM to 10 mM. In some embodiments, the concentration is in a range of from 1 mM to 5 mM (e.g., 3 mM).

In some of any of the embodiments described herein or in any combination thereof, a total amount of the at least one bilayer-forming lipid in the liposome ranges from 50 to 99 mol %, including any intermediate values and subranges therebetween.

In some of any of the embodiments described herein, an amount of the polymeric compound (LPC) ranges from 0.1 to 10, or from 0.1 to 5, or from 0.1 to 3, or from 0.1 to 1, or from 0.2 to 0.8, mol % of the total amount of lipids in the liposomes, including any intermediate values and

In some of any of the embodiments described herein, an amount of the polymeric compound (LPC) ranges from 1 to 50, or from 1 to 40, or from 1 to 30, or from 5 to 50, or from 5 to 40, or from 5 to 30, % by weight of the total amount of lipids in the liposomes, including any intermediate values and subranges therebetween, the remaining being the bilayer forming lipid(s).

In some of any of the embodiments described herein, a mean diameter of liposomes ranges from about 100 nm to about 2000 nm, or from about 100 nm to about 1000 nm, of from about 100 nm to about 500 nm, or from about 100 nm to about 200 nm, or from about 150 nm to about 200 nm, or from about 150 nm to about 180 nm, including any intermediate values and subranges therebetween.

The mean diameter according to any of the respective embodiments described herein may optionally be an arithmetic mean (a ratio of a sum of values to the number of values) or a Z-average as this term is defined in the art of dynamic light scattering (in brief, an intensity-weighted harmonic mean). In exemplary embodiments, the mean diameter is a Z-average diameter determined by dynamic light scattering.

The number-average molecular weight (Mn) and/or molecular weight (Mw) and/or number-average degree of polymerization (DPn) of liposomes in a composition may optionally be determined by gel permeation chromatography (GPC) analysis.

The polydispersity index (PDI) and/or mean diameter of liposomes in a composition may optionally be determined by dynamic light scattering using a two-parameter fit to the data (e.g., according to ISO 13321 and ISO 22412 standards) for determining PDI and Z-average diameter (e.g., using a commercially available instrument).

In some of any of the embodiments described herein, a PDI of the liposomes is lower than 1.

In some of any of the embodiments described herein, a zeta potential of the liposomes is in a range of from 10 mV to −50 mV, or from 0 mV to −50 mV, or from 0 mV to −30 mV, or from −5 mV to −25 mV, or from −10 mV to −25 mV, including any intermediate values and subranges therebetween.

In some of any of the embodiments described herein, a zeta potential of the liposomes is at least −3 mV (i.e., −3 mV or a more negative value), optionally at least −3.5 mV, and optionally at least −4 mV.

In some of any of the embodiments described herein relating to a polymeric compound comprising a negatively-charged bilayer-forming lipid (e.g., DPPG), the zeta potential of the liposomes is in a range of from 10 mV to −10 mV (e.g., from 5 mV to −5 mV), optionally in a range of from 0 to −10 mV (e.g., from 0 to −5 mV, or from −3 mV to −5 mV).

Zeta potential may optionally be determined using any suitable technique known in the art (e.g., using a commercially available instrument), for example, electrophoretic light scattering. The zeta potential of liposomes may be determined by diluting the liposomes in an aqueous salt (e.g., NaCl) solution with a predetermined salt concentration (e.g., 10 μM).

In some of any of the embodiments described herein, the bilayer-forming lipid or the liposome comprises at least one zwitterionic bilayer-forming lipid, for example, a zwitterionic glycerophospholipid.

In some of any of the embodiments described herein, the bilayer-forming lipid or the liposome comprises at least one zwitterionic bilayer-forming lipid, for example, a zwitterionic glycerophospholipid, and at least one negatively-charged bilayer-forming lipid, for example, a negatively charged phosphatidyl glycerol, in accordance with any of the embodiments described herein.

In some of any of the embodiments described herein or in any combination thereof, the at least one negatively-charged bilayer-forming lipid is a phosphatidyl glycerol (e.g., DPPG).

In some of any of the embodiments described herein or in any combination thereof, the at least one bilayer-forming lipid further comprises at least one zwitterionic glycerophospholipid (e.g., a phosphatidyl choline such as DSPC and/or or a phosphatidyl ethanolamine such as DPPE).

Herein and in the art, the term “phosphatidylcholine” refers to a glycerophospholipid comprising a phosphocholine group and two fatty acyl groups attached to a glycerol backbone (i.e., a diacylglyceride).

Herein and in the art, the term “phosphatidylethanolamine” refers to a glycerophospholipid comprising a phosphoethanolamine group and two fatty acyl groups attached to a glycerol backbone (i.e., a diacylglyceride).

According to some embodiments of any of the embodiments of the invention, the bilayer-forming lipid comprises a negatively-charged bilayer-forming lipid (e.g., a phosphatidyl glycerol such as DPPG).

According to some embodiments of any of the embodiments of the invention, the bilayer-forming lipid comprises a zwitterionic glycerophospholipid (e.g., a phosphatidyl choline such as DSPC, or a phosphatidyl ethanolamine such as DPPE) and a negatively-charged glycerophospholipid (e.g., a phosphatidyl glycerol such as DPPG). According to some embodiments of any of the embodiments of the invention, the total amount of the bilayer-forming lipids in the liposomes ranges from 50 to 99 mol %, including any intermediate values and

According to some embodiments of any of the embodiments of the invention, an amount of the negatively-charged bilayer-forming lipid, if present, ranges from 0.1 to 40 mol %, or from 0.1 to 20 mol %, or from 0.1 to 20 mol %, of the lipids in the liposomes (bilayer-forming lipids, LPC and cholesterol, if present), including any intermediate values and subranges therebetween.

According to some embodiments of any of the embodiments of the invention, the liposome further comprises a sterol, for example, cholesterol.

According to some embodiments of any of the embodiments of the invention, the sterol (e.g., cholesterol) is associated with the lipid bilayer (but does not form the lipid bilayer).

As used herein, the term “sterol” encompasses all sterols derived from any source and includes synthetic sterols, animal-derived sterols, and plant-derived sterols (termed “phytosterols” as known in the art), as well as the saturated forms of sterols thereof (i.e., stanols). Thus, the term “sterols” as used herein encompasses both sterols and stanols. Sterols are steroids with a hydroxyl group at C₃ (steroid alcohol) and most of the skeleton of cholestane (IUPAC Steroid Nomenclature, 1987). Additional carbon atoms may be present in the side chain, usually in the C₁₇ position. In nature, sterols are found as C₂₆-C₃₀ steroid alcohols. The cylcopentanoperhydrophenanthrene ring structure is common to all sterols, while the side chains may vary in structure. In nature, sterols may be found as conjugates (e.g., glycoconjugates, lipo-conjugates, etc.). Thus, the term “sterol” as used herein, is further intended to encompass conjugated sterols, including, but not limited to, phytosteryl fatty-acid esters and phytostanyl fatty-acid esters. An exemplary sterol is cholesterol. According to some embodiments of any of the embodiments of the invention, an amount of the sterol (e.g., cholesterol) ranges from 0.1 to 50 mol % of the total lipids in the liposomes, including any intermediate values and subranges therebetween.

According to some embodiments of any of the embodiments of the invention, an amount of the polymeric compound (LPC) ranges from 0.1 to 10, or from 0.1 to 5, or from 0.1 to 1, mol % of the total amount of lipids in the liposomes, including any intermediate values and subranges therebetween.

In some of any of the embodiments described herein or in any combination thereof, an amount of the at least one negatively-charged bilayer-forming lipid, if present, ranges from 0.1 to 40 mol %, or from 0.1 to 20 mol %, of the lipids in the liposomes, including any intermediate values and subranges therebetween.

In some embodiments of any of the embodiments described herein relating to a liposome, the liposome further comprises at least one functional moiety or agent bound to or associated with a surface of the liposome and/or within a lipid bilayer and/or core of the liposome (e.g., within the liposome bilayer and/or enveloped by the liposome bilayer). In exemplary embodiments, a functional moiety is bound to the liposome, for example, it is covalently bound to the liposome by means of, for example, covalent bonding with one or more the lipids. In exemplary embodiments, a functional agent is associated with the liposome either chemically, by means of, for example, covalent or electrostatic bonds, and/or physically, by being entrapped or entangled within the lipid bilayer or the core.

Examples of functional moieties and agents suitable for inclusion in embodiments described herein include, without limitation, a therapeutically active agent or moiety of a therapeutically active agent (e.g., wherein the active agent is releasable upon cleavage of the moiety), a labeling moiety or agent, and/or a targeting moiety or targeting agent (e.g., a targeting moiety or agent on a surface of the liposome). Any other moiety or agent that may contribute to or improve the indicated use of the liposome is contemplated. According to some embodiments, the liposome further comprises a sterol, for example, cholesterol. According to some of these embodiments, the cholesterol is incorporated in and/or associated with the lipid bilayer.

According to some of any of the embodiments described herein, the functional moiety or agent is a therapeutically active agent or moiety thereof, a labeling moiety or agent and/or a targeting moiety or agent.

According to some of any of the embodiments described herein, the functional moiety or agent is a therapeutically active agent or moiety thereof, and in some of these embodiments, the therapeutically active agent or moiety thereof is within the lipid bilayer and/or core of the liposome. According to some of these embodiments, the liposome further comprises a sterol, for example, cholesterol, in an amount of from 0.1 to 50 mol % of the total lipids in the liposome. According to some of these embodiments, the polymeric compound is a long polymeric compound, as described and defined herein in any of the respective embodiments.

According to some embodiments, the liposome comprises a therapeutically active agent, bound to or associated with a surface of the liposome and/or within a lipid bilayer and/or core of the liposome (e.g., within the liposome bilayer and/or enveloped by the liposome bilayer), and cholesterol (e.g., associated within the lipid bilayer).

In some of any of the embodiments described herein, the liposomes are devoid of a therapeutically active agent.

In some of any of the embodiments described herein, liposomes comprise a therapeutically active agent, which is optionally incorporated in a liposome and/or on a surface of the liposome. In some such embodiments, the therapeutically active agent is a therapeutically active agent described in International Patent Application publication WO 2018/150429.

Herein, the phrase “therapeutically active agent” refers to any agent (e.g., compounds) having a therapeutic effect, provided that the compound is not a bilayer-forming lipid or polymeric compound comprised by the liposome (according to any of the respective embodiments described herein), as well as to any portion of an agent (e.g., a moiety of a compound) which generates an agent having a therapeutic effect upon release (e.g., upon cleavage of one or more covalent bonds), including a portion of a bilayer-forming lipid or polymeric compound. Thus, the bilayer-forming lipid and polymeric compound per se are excluded from the definition of a therapeutically active agent, but a bilayer-forming lipid and/or polymeric compound may optionally generate a therapeutically active agent upon release, in which case the portion of the bilayer-forming lipid and/or polymeric compound which generates the therapeutically active agent is also considered to be a therapeutically active agent as defined herein.

When associated with a liposome, a therapeutically active agent may optionally be attached by a covalent or non-covalent (e.g., electrostatic and/or hydrophobic) bond to a liposome (e.g., to an exterior surface and/or interior surface of a liposome membrane), incorporated within a liposome membrane (e.g., a lipophilic agent which stably partitions to a lipid phase of the liposome), and/or enveloped within a core of a liposome (e.g., a hydrophilic agent in an aqueous compartment of the liposome). The therapeutically active agent may optionally be a moiety covalently attached to a liposome (e.g., attached to a lipid so as to form a lipid-derivative comprising the moiety). Such attachment may be obtained in some embodiments by using techniques known in the art (e.g., amide bond formation).

In some embodiments, a therapeutically active agent is attached to the liposome via electrostatic interactions, for example, the therapeutically active agent is a positively-charged agent which is attached to, or complexed by, the negatively-charged bilayer-forming lipid as described herein.

In some embodiments of any of the embodiments described herein, the therapeutically active agent is, for example, an analgesic, an anti-inflammatory agent, an anti-proliferative agent, an anti-microbial agent (including antibacterial, anti-mycobacterial, antiviral, anti-fungal, anti-protozoal and/or anti-parasitic agents) and/or a vaccine antigen. In some such embodiments, the therapeutically active agent is an analgesic and/or an anti-inflammatory agent. In some such embodiments, the therapeutically active agent is usable in the treatment osteoarthritis, either alone or in combination with an additional therapeutically active agent.

The phrase “anti-microbial” as used herein, refers to a property of a substance (e.g., a compound or a composition) that can effect a parameter of microorganism, as defined herein, including death, eradication, elimination, reduction in number, reduction of growth rate, inhibition of growth, and change in population distribution of one or more species of microbial life forms. This term encompasses antibacterial agents, which are also referred to herein as antibiotics. Examples of anti-microbial agents include, without limitation, antibacterial, anti-mycobacterial, antiviral, anti-fungal, anti-protozoal and/or anti-parasitic agents, as known in the art.

The phrase “vaccine antigen” as used herein, refers to an agent within a vaccine that can stimulate the immune system to recognize and respond to a specific pathogen or foreign agent. Upon introduction into a patient's body, the antigen triggers an immune response which lead to the production of antibodies within the body of the patient. Examples of vaccine antigens include, without limitation, inactivated or subunit vaccines (e.g., influenza vaccine; hepatitis B vaccine), live attenuated vaccines (e.g., yellow fever vaccine), viral vector vaccines (e.g., COVID-19 vaccines), toxoid vaccines (e.g., tetanus vaccine), and/or conjugate vaccines (Haemophilus influenzae type b (Hib) vaccine).

Examples of suitable anti-proliferative agents include, without limitation, acivicin; aclarubicin; acodazole (e.g., acodazole hydrochloride); acronine; adriamycin; adozelesin; aldesleukin; altretamine; ambomycin; ametantrone (e.g., ametantrone acetate); aminoglutethimide; amsacrine; anastrozole; anthramycin; asparaginase; asperlin; azacitidine; azetepa; azotomycin; batimastat; benzodepa; bicalutamide; bisantrene (e.g., bisantrene hydrochloride); bisnafide (e.g., bisnafide dimesylate); bizelesin; bleomycin (e.g., bleomycin sulfate); brequinar (e.g., brequinar sodium); bropirimine; busulfan; cactinomycin; calusterone; caracemide; carbetimer; carboplatin; carmustine; carubicin (e.g., carubicin hydrochloride); carzelesin; cedefingol; chlorambucil; cirolemycin; cisplatin; cladribine; combrestatin A-4 phosphate; crisnatol (e.g., crisnatol mesylate); cyclophosphamide; cytarabine; dacarbazine; dactinomycin; daunorubicin (e.g., daunorubicin hydrochloride); decitabine; dexormaplatin; dezaguanine (e.g., dezaguanine mesylate); diaziquone; docetaxel; doxorubicin (e.g., doxorubicin hydrochloride); droloxifene (e.g., droloxifene citrate); dromostanolone (e.g., dromostanolone propionate); duazomycin; edatrexate; eflornithine (e.g., eflornithine hydrochloride); elsamitrucin; enloplatin; enpromate; epipropidine; epirubicin (e.g., epirubicin hydrochloride); erbulozole; esorubicin (e.g., esorubicin hydrochloride); estramustine (e.g., estramustine phosphate sodium); etanidazole; etoposide (e.g., etoposide phosphate); etoprine; fadrozole (e.g., fadrozole hydrochloride); fazarabine; fenretinide; floxuridine; fludarabine (e.g., fludarabine phosphate); fluorouracil; flurocitabine; fosquidone; fostriecin (e.g., fostriecin sodium); gemcitabine (e.g., gemcitabine hydrochloride); hydroxyurea; idarubicin (e.g., idarubicin hydrochloride); ifosfamide; ilmofosine; interferon alfa-2a; interferon alfa-2b; interferon alfa-n1; interferon alfa-n3; interferon beta-Ia; interferon gamma-Ib; iproplatin; irinotecan (e.g., irinotecan hydrochloride); lanreotide (e.g., lanreotide acetate); letrozole; leuprolide (e.g., leuprolide acetate); liarozole (e.g., liarozole hydrochloride); lometrexol (e.g., lometrexol sodium); lomustine; losoxantrone (e.g., losoxantrone hydrochloride); masoprocol; maytansine; mechlorethamine (e.g., mechlorethamine hydrochloride); megestrol (e.g., megestrol acetate); melengestrol (e.g., melengestrol acetate); melphalan; menogaril; mercaptopurine; methotrexate (e.g., methotrexate sodium); metoprine; meturedepa; mitindomide; mitocarcin; mitocromin; mitogillin; mitomalcin; mitomycin; mitosper; mitotane; mitoxantrone (e.g., mitoxantrone hydrochloride); mycophenolic acid; nocodazole; nogalamycin; ombrabulin; ormaplatin; oxisuran; paclitaxel; pegaspargase; peliomycin; pentamustine; peplomycin (e.g., peplomycin sulfate); perfosfamide; pipobroman; piposulfan; piroxantrone (e.g., piroxantrone hydrochloride); plicamycin; plomestane; porfimer (e.g., porfimer sodium); porfiromycin; prednimustine; procarbazine (e.g., procarbazine hydrochloride); puromycin (e.g., puromycin hydrochloride); pyrazofurin; riboprine; rogletimide; safingol (e.g., safingol hydrochloride); semustine; simtrazene; sparfosate (e.g., sparfosate sodium); sparsomycin; spirogermanium (e.g., spirogermanium hydrochloride); spiromustine; spiroplatin; streptonigrin; streptozocin; sulofenur; talisomycin; tecogalan (e.g., tecogalan sodium); tegafur; teloxantrone (e.g., teloxantrone hydrochloride); temoporfin; teniposide; teroxirone; testolactone; thiamiprine; thioguanine; thiotepa; tiazofurin; tirapazamine; topotecan (e.g., topotecan hydrochloride); toremifene (e.g., toremifene citrate); trestolone (e.g., trestolone acetate); triciribine (e.g., triciribine phosphate); trimetrexate (e.g., trimetrexate glucoronate); triptorelin; tubulozole (e.g., tubulozole hydrochloride); uracil mustard; uredepa; vapreotide; verteporfin; vinblastine; vincristine (e.g., vincristine sulfate); vindesine (e.g., vindesine sulfate); vinepidine; vinglycinate; vinleurosine; vinorelbine (e.g., vinorelbine tartrate); vinrosidine; vinzolidine; vorozole; zeniplatin; zinostatin; and zorubicin (e.g., zorubicin hydrochloride). Additional anti-cancer agents include those disclosed in Chapter 52, Antineoplastic Agents (Paul Calabresi and Bruce A. Chabner), and the introduction thereto, 1202-1263, of Goodman and Gilman's “The Pharmacological Basis of Therapeutics”, Eighth Edition, 1990, McGraw-Hill, Inc. (Health Professions Division), the contents of which are incorporated herein by reference.

In some of any of the embodiments described herein, a sterol, e.g., cholesterol, is included in the lipid bilayer of the liposome, in accordance with any of the embodiments described herein.

In some of any of the embodiments described herein, an amount of the sterol (e.g., cholesterol) ranges from 0.1 to 50 mol % of the total lipids in the liposomes, including any intermediate values and subranges therebetween. Examples of therapeutically active agents suitable for inclusion in a liposome (e.g., as a molecule or moiety of the agent) include, without limitation, amphotericin B, cisplatin, cytarabine, daunorubicin, doxorubicin, estradiol, influenza virosome, morphine, surfactant protein B, surfactant protein C, verteporfin and vincristine.

Examples of a labeling moiety or agent include moieties and compounds which are chromophoric (e.g., absorb visible light), fluorescent, phosphorescent, and/or radioactive. Many such compounds and moieties (and techniques for preparing such moieties) will be known to a skilled person.

A targeting moiety in a liposome according to any of the respective embodiments described herein may optionally be a targeting moiety according to any of the respective embodiments described herein. A targeting moiety in a liposome may be comprised by a polymeric compound according to some embodiments of the invention (according to any of the respective embodiments described herein), the liposome comprising the polymeric compound. Alternatively or additionally, a targeting moiety in a liposome may optionally be comprised by another compound in the liposome, optionally a bilayer-forming lipid (according to any of the respective embodiments described herein) conjugated to a targeting moiety according to any of the respective embodiments described herein.

Herein, a “targeting agent” refers to a compound (“agent”) comprising (and optionally consisting essentially of) a targeting moiety according to any of the respective embodiments described herein (e.g., in the context of a targeting moiety comprised by a polymeric compound described herein). Typically, the phrase “targeting agent” is used to refer to a compound other than a polymeric compound comprising a targeting moiety, as described herein.

In some embodiments, a functional moiety (e.g., targeting moiety or labeling moiety) is covalently attached to a liposome. Such attachment may be obtained in some embodiments by using techniques known in the art (e.g., amide bond formation).

### Compositions-of-Matter and Articles:

According to another aspect of embodiments of the invention, there is provided a composition-of-matter comprising a substrate coated, on at least a portion of a surface thereof, by at lipid bilayer according to any of the respective embodiments described herein.

According to another aspect of embodiments of the invention, there is provided an article of manufacture comprising a composition-of-matter according to any one of the embodiments described herein.

Herein, the term “composition-of-matter” refers to any composition comprising a plurality of substances (e.g., substrate, water-soluble polymer(s), and amphiphilic lipid) in a form which does not exist in nature, and which does not include a portion of a human being. The form which does not exist in nature may optionally comprise natural substances in a combination which does not exist in nature, and/or may optionally comprise one or more substances which do not occur in nature. It is to be understood that this definition is not necessarily identical with a standard legal definition of the term.

Herein, the term “article of manufacture” refers to any article produced from materials in a manner which results in new forms, qualities, properties or combinations of the materials. It is to be understood that this definition is not necessarily identical with a standard legal definition of the term. The article of manufacture described herein may optionally consist essentially of the composition-of-matter, or alternatively, may comprise additional materials and/or parts.

At least a portion of the molecules of the amphiphilic lipid are oriented such that polar groups thereof (e.g., charged groups) face outwards at a surface of the composition-of-matter.

As used herein, the phrase “face outwards at a surface” refers to a group in a molecule (e.g., a lipid) which is closer to the surface of the composition-of-matter than the center of gravity of the molecule is to the surface of the composition-of-matter, and farther from the substrate than the center of gravity of the molecule is from the substrate.

As discussed herein, and without being bound by any particular theory, it is believed that outwards facing polar groups (e.g., charged groups) according to some embodiments of the invention are effect highly effective lubrication and/or inhibition of adhesion, biofouling and/or biofilm formation (e.g., as described herein) due, at least in part, to properties of hydrated polar groups (e.g., hydration lubrication), especially hydrated charged groups.

In any of the embodiments described herein, the substrate may comprise any type of material or combination of different types of material, including inorganic material and/or organic material, in crystalline, amorphous and/or gel (e.g., hydrogel) forms, for example, metal, mineral, ceramic, glass, polymer (e.g., synthetic polymer, biopolymer), plant and/or animal biomass, and combinations thereof.

In some embodiments, the substrate comprises a physiological surface (e.g., a physiological tissue) and/or a surface in contact with and/or intended to come into contact with a physiological surface (e.g., as described herein in any one of the respective embodiments).

In some embodiments of any of the embodiments described herein, the article of manufacture is a medical device, e.g., a medical device having lipids attached to at least a portion of a surface thereof. In some embodiments, the medical device is a device designed to come into contact with a part of the body susceptible to infection, such as an internal portion of the body, a mucous membrane and/or a surface of the eye. Examples of such medical devices include, without limitation, surgical tools and implants (which are for coming into contact with an internal portion of the body) and contact lenses (which are for contacting a surface of the eye).

As used herein throughout, the phrase “medical device” includes any material or device that is used on, in, or through a subject's body, for example, in the course of medical treatment (e.g., for a disease or injury). The subject may be human or a non-human animal, such that the phrase “medical device” encompasses veterinary devices. Medical devices include, but are not limited to, such items as medical implants (including permanent implants and transient implants), wound care devices, medical devices for drug delivery, contact lenses and body cavity and personal protection devices. The medical implants include, but are not limited to, catheters (e.g., urinary catheters, intravascular catheters), injection ports, intubation equipment, dialysis shunts, wound drain tubes, skin sutures, vascular grafts, implantable meshes, intraocular devices, heart valves, and the like. Wound care devices include, but are not limited to, general wound dressings, biologic graft materials, tape closures and dressings, and surgical incise drapes. Medical devices for drug delivery include, but are not limited to, needles, drug delivery skin patches, drug delivery mucosal patches and medical sponges. Body cavity and personal protection devices, include, but are not limited to, tampons, sponges, surgical and examination gloves, and toothbrushes. Birth control devices include, but are not limited to, intrauterine devices (IUDs), diaphragms and condoms.

In the context of medical devices, it is to be understood that the medical device is coated by a bilayer described herein, and a lipid bilayer or bilayer-comprising liposome per se is not considered herein to be a medical device.

Examples of suitable articles of manufacture include, without limitation:
medical devices (e.g., contact lenses, pacemakers, heart valves, replacement joints, catheters, catheter access ports, dialysis tubing, gastric bands, shunts, screw plates, artificial spinal disc replacements, internal implantable defibrillators, cardiac resynchronization therapy devices, implantable cardiac monitors, mitral valve ring repair devices, left ventricular assist devices (LVADs), artificial hearts, implantable infusion pumps, implantable insulin pumps, stents, implantable neurostimulators, maxillofacial implants, dental implants, and the like);
packages or containers, for example, packages or containers for food and/or beverages (e.g., packages for meat and/or dairy products and/or containers for storage or transportation of meat and/or dairy products, such as storage tanks, raw milk holding equipment, dairy processing operations conveyer belts, tube walls, gaskets, rubber seals, stainless steel coupons, piping systems, filling machine, silo tanks, heat exchangers, post-pasteurization equipment, pumps, valves, separators, and spray devices), medical device packages, agricultural packages and containers (of agrochemicals), blood sample or other biological sample packages and containers, and any other packages or containers of various articles; and elements of water treatment systems (e.g., for containing and/or transporting and/or treating aqueous media or water), devices, containers, filters, tubes, solutions and gases and the like.

In the context of medical devices, it is to be understood that the medical device is coated by a bilayer described herein, and a bilayer or bilayer-comprising liposome per se is not considered herein to be a medical device.

Exemplary articles include the following:
Medical devices such as, but not limited to, pacemakers, heart valves, replacement joints, catheters, catheter access ports, dialysis tubing, gastric bands, shunts, screw plates, artificial spinal disc replacements, internal implantable defibrillators, cardiac resynchronization therapy devices, implantable cardiac monitors, mitral valve ring repair devices, left ventricular assist devices (LVADs), artificial hearts, implantable infusion pumps, implantable insulin pumps, stents, implantable neurostimulators, maxillofacial implants, dental implants, and the like;
Packages or containers, for example, food packages and containers, beverage packages and containers, medical device packages, agricultural packages and containers (of agrochemicals), blood sample or other biological sample packages and containers, and any other packages or containers of various articles;
Food packages such as packages of dairy products and/or containers for storage or transportation of dairy products;
Milk storage and processing devices such as, but not limited to, containers, storage tanks, raw milk holding equipment, dairy processing operations conveyer belts, tube walls, gaskets, rubber seals, stainless steel coupons, piping systems, filling machine, silo tanks, heat exchangers, post-pasteurization equipment, pumps, valves, separators, and spray devices;
Energy harvesting device, for example, a microelectronic device, a microelectromechanical device, a photovoltaic device and the like;
Microfluidic devices, for example, micro-pumps or micro valves and the like; Sealing parts, for example, O rings, and the like; Articles having a corrodible surface;
Agricultural devices, as, for example, described herein;
Textiles, for example, tough cottons;
Fuel transportation devices;
Construction elements, such as, but not limited to, paints, walls, windows, door handles, and the like;
Elements of water treatment systems (such as for containing and/or transporting and/or treating aqueous media or water), devices, containers, filters, tubes, solutions and gases and the like; and
Elements of organic waste treatment systems (such as for containing and/or disposing and/or transporting and/or treating organic waste), devices, containers, filters, tubes, solutions and gases and the like.

In some of any one of the embodiments described herein, the article of manufacture comprises a hydrogel surface, e.g., having lipids attached to at least a portion thereof.

Contact lenses are an exemplary article of manufacture comprising a hydrogel surface. In some embodiments, the contact lens comprises a hydrogel surface and a rigid center. In some embodiments, the contact lens consists essentially of a hydrogel.

The hydrogel may comprise any material known in the art for use in contact lens hydrogels. Examples of such hydrogel materials include, without limitation, alphafilcon A, asmofilcon A, balafilcon A, bufilcon A, comfilcon A, crofilcon, deltafilcon A, dimefilcon, droxifilcon A, enfilcon A, ctafilcon A, galyfilcon A, hefilcon A, hefilcon B, hilafilcon A, hilafilcon B, hioxifilcon A, hioxifilcon D, isofilcon, lidofilcon A, lidofilcon B, lotrafilcon B, mafilcon, methafilcon A, methafilcon B, narafilcon A, narafilcon B, ocufilcon A, ocufilcon B, ofilcon A, omafilcon A, perfilcon, phemfilcon A, polymacon, scafilcon A, senofilcon A, surfilcon, tefilcon, tetrafilcon A, tetrafilcon B, vifilcon A, and xylofilcon A.

In some embodiments of any one of the embodiments described herein, the hydrogel comprises a polymer consisting of poly(2-hydroxyethyl methacrylate) and/or a silicone. In some embodiments, the polymer comprises a silicone. Such polymers may optionally comprise small amounts of additional monomers (e.g., cross-linking monomers) copolymerized with the 2-hydroxyethyl methacrylate or silicone monomer. For example, 2-hydroxyethyl methacrylate may optionally be copolymerized with vinyl pyrrolidone, methyl methacrylate, methacrylic acid (an anionic monomer), ethylene glycol dimethacrylate (a cross-linking monomer) and/or 3-(ethyldimethyl-ammonium)propyl methacrylamide (a cationic monomer) in a contact lens hydrogel.

### Sterile Compositions:

According to an aspect of some embodiments of the invention, there is provided a sterile composition comprising an aqueous carrier and a lipid bilayer a liposomes, wherein the lipid bilayer of the liposomes comprise at least one bilayer-forming lipid and a polymeric compound according to any of the respective embodiments described herein.

Herein, the term “sterile” refers to an absence of observable microorganism growth upon subjecting composition to conditions (e.g., medium, incubation temperature) for a suitable period of time, e.g., according to any standard protocol for testing sterility. Optionally, sterility is tested in fluid thioglycollate medium, e.g., at a temperature in a range of from 30 to 35° C. for up to 3 days, and/or soybean-casein digest medium (a.k.a. trypticase soy broth or trypticase soy agar), e.g., at a temperature in a range of from 20 to 25° C. for up to 5 days; for example, whereby a composition is sterile if no microbial growth is observable in either medium. The fluid thioglycollate medium and/or soybean-casein digest medium content and/or procedures for testing sterility may optionally be as described in U.S. Pharmacopeia General Chapter 71, the contents of which are incorporated herein by reference.

In some of any of the embodiments described herein, a sterile composition is further characterized by a concentration of bacterial endotoxins below an acceptable threshold, for example below a threshold of 35 endotoxin units (EU) per ml (e.g., wherein endotoxin units are defined according to the U.S. Pharmacopeia reference standard). Bacterial endotoxin level may be determined by any suitable test known in the art, for example, using horseshoe crab (Limulus) amebocyte lysate (e.g., according to Chapter 85 of the U.S. Pharmacopeia, the contents of which are incorporated herein by reference), for example, by comparing with commercially available reference samples containing endotoxins.

In some of any of the respective embodiments, the sterile composition comprises an article of manufacture immersed therein (which, being part of the sterile composition, is also sterile), such as a solid or semi-solid article of manufacture which is commonly packaged together with a liposome-containing aqueous composition. In some embodiments, the article of manufacture is a contact lens, for example, wherein the aqueous carrier and liposomes represent a contact lens storage solution.

The sterile composition according to any of the respective embodiments described herein may optionally be prepared according to a process described herein according to any of the respective embodiments.

According to an aspect of some embodiments of the invention, there is provided a process for preparing a sterile composition comprising an aqueous carrier (e.g., according to any of the respective embodiments described herein) and liposomes (e.g., according to any of the respective embodiments described herein). The process comprises providing an aqueous composition comprising the aqueous carrier and liposomes which comprise at least one bilayer-forming lipid (e.g., according to any of the respective embodiments described herein) and a polymeric compound (e.g., according to any of the respective embodiments described herein); and subjecting the aqueous composition to a temperature of over 100° C.

The sterile composition obtained according to the process may optionally be a sterile composition according to any of the respective embodiments described herein.

In some of any of the respective embodiments described herein, the aqueous composition comprising an aqueous carrier and liposomes further comprises an article of manufacture immersed therein (e.g., according to any of the respective embodiments described herein), such that upon subjecting the aqueous composition to a temperature of over 100° C., the article of manufacture immersed in the aqueous composition becomes sterile. In some exemplary embodiments, the article of manufacture comprises a contact lens. Such a process may allow for efficient and relatively low-cost simultaneous sterilization of solid or semi-solid articles of manufacture and liposome-containing aqueous composition (e.g., which are commonly packaged together), for example, one or more contact lenses immersed in a liposome-containing contact lens solution (e.g., contact lens storage solution).

According to another aspect of some embodiments of the invention, there is provided a process for preparing a sterile article of manufacture having lipids attached to at least a portion of a surface thereof, the process comprising contacting at least a portion of a surface of the article of manufacture with an aqueous composition comprising an aqueous carrier (e.g., according to any of the respective embodiments described herein) and liposomes (e.g., according to any of the respective embodiments described herein), to thereby obtain an article of manufacture having lipids attached to at least a portion of a surface thereof; and subjecting the article of manufacture having lipids attached to at least a portion of surface thereof to a temperature of over 100° C. The liposomes comprise at least one bilayer-forming lipid (e.g., according to any of the respective embodiments described herein) and a polymeric compound (e.g., according to any of the respective embodiments described herein). In some exemplary embodiments, the article of manufacture comprises a contact lens.

It is to be appreciated that according to this aspect, the lipids attached to at least a portion of the surface may be in a form of liposomes and/or in another form, such as an open bilayer (i.e., a bilayer which does not enclose a volume), e.g., obtainable by “bursting” of the liposomes upon contact with the surface. Optionally, at least a portion of the lipids attached to the surface may optionally be in a different form prior to application of a temperature of over 100° C. than subsequent to application of a temperature of over 100° C., for example, in a form of liposomes prior to sterilization by heating and in a different form (e.g., an open bilayer) subsequent to sterilization by heating. Alternatively or additionally, the form of the lipid changes gradually (e.g., from liposomes to another form) upon incubation of the article of manufacture in the aqueous composition subsequent to sterilization by heating (e.g., over the course of at least one hour or at least one day, or even at least one month after sterilization).

Such a process may allow for efficient and relatively low-cost sterilization of solid or semi-solid articles of manufacture which has lipids attached to at least a portion of a surface thereof, for example, one or more contact lenses coated with a lipid.

In some of any of the respective embodiments described herein, according to any of the aspects described herein, the temperature to which the aqueous composition is subjected is no more than 150° C., for example, from 110° C. to 150° C., or from 115° C. to 150° C., or from 121° C. to 150° C., or from 130° C. to 150° C.

In some of any of the respective embodiments described herein, the temperature to which the aqueous composition is subjected is no more than 140° C., for example, from 110° C. to 140° C., or from 115° C. to 140° C., or from 121° C. to 140° C., or from 130° C. to 140° C.

In some of any of the respective embodiments described herein, the temperature to which the aqueous composition is subjected is no more than 134° C., for example, from 110° C. to 134° C., or from 115° C. to 134° C., or from 121° C. to 134° C.

In some of any of the respective embodiments described herein, the temperature to which the aqueous composition is subjected is no more than 130° C., for example, from 110° C. to 130° C., or from 115° C. to 130° C., or from 121° C. to 130° C.

In some of any of the respective embodiments described herein, the temperature to which the aqueous composition is subjected is no more than 125° C., for example, from 110° C. to 125° C., or from 115° C. to 125° C., or from 121° C. to 125° C.

In some of any of the respective embodiments described herein, subjecting the aqueous composition to a temperature of over 100° C. is effected at an elevated pressure, i.e., a pressure greater than ambient atmospheric pressure. Such a pressure may be obtained, for example, by heating the aqueous composition in a closed vessel, such that water vapor formed by the heating participates in elevating the pressure. In some such embodiments, the pressure is such that the boiling point of the aqueous composition at that pressure is equal to the temperature or near (e.g., ±10° C. or ±5° C.) the temperature to which the composition is subjected (according to any of the respective embodiments described herein).

Subjecting the composition to an elevated temperature (and optionally elevated pressure) according to any of the respective embodiments described herein may optionally be effected using a commercial apparatus configured for such use, such as an autoclave.

According to some of any of the embodiments described herein, the sterile composition is obtained or is obtainable by a process as described herein in any of the respective embodiments and any combination thereof.

### Lubrication:

Liposomes and lipid bilayers described herein may optionally be useful for lubricating a surface, for example, a surface coated by a bilayer described herein, and/or contacted with liposomes described herein.

According to an aspect of some embodiments of the invention, there is provided a lubricant composition comprising liposomes or lipid bilayers according to any of the respective embodiments described herein.

Herein, a “lubricant composition” refers to a composition intended for reducing a friction coefficient of a surface (e.g., according to a method described herein).

In some embodiments, the lubricant composition comprises a carrier. The carrier may optionally be a liquid carrier. In some embodiments, the carrier comprises an aqueous liquid.

In some embodiments, the lubricant composition (or any other composition or formulation descried herein comprising liposomes) further comprises a water-soluble polymer, optionally as part of the carrier.

As used herein, the phrase “water-soluble polymer” encompasses polymers having a solubility of at least 1 gram per liter in an aqueous (e.g., water) environment at pH 7 (at 25° C.).

In some embodiments of any of the embodiments described herein, the water-soluble polymer has a solubility of at least 2 grams per liter (under the abovementioned conditions). In some embodiments, the solubility is at least 5 grams per liter. In some embodiments, the solubility is at least 10 grams per liter. In some embodiments, the solubility is at least 20 grams per liter. In some embodiments, the solubility is at least 50 grams per liter. In some embodiments, the solubility is at least 100 grams per liter.

The water-soluble polymer(s) according to any of the embodiments described herein may comprise at least one ionic polymer and/or at least one non-ionic polymer which are water-soluble as defined herein.

As used herein, the phrase “non-ionic polymer” refers to a polymer which does not have a charged group. Examples of suitable non-ionic water-soluble polymers include, without limitation, polyvinylpyrrolidone (also referred to herein interchangeably as povidone and/or PVP) and polyethylene oxide (also referred to herein interchangeably as PEO, PEG and/or polyethylene glycol).

As used herein, the phrase “ionic polymer” refers to polymers having at least one charged group, and encompasses polymers having a net negative charge (also referred to herein as “anionic polymers”), polymers having a net positive charge (also referred to herein as “cationic polymers”), and polymers having no net charge (also referred to herein as “zwitterionic polymers”), in an aqueous (e.g., water) environment at pH 7.

Herein throughout, the phrase “charged group” refers to any functional group (e.g., a functional group described herein) which is ionic (as defined herein), including, for example, amine, carboxylic acid, sulfate, sulfonate, phosphate and phosphonate. Thus, each electric charge in a moiety or molecule is associated with one charged group, although a single charged group (e.g., non-substituted phosphate) may be associated with more than one electric charge of the same sign (e.g., a dianion, a dication).

Herein throughout, the term “ionic” refers to the presence of an electric charge on at least one atom in a moiety and/or molecule (in at least 50% of moieties and/or molecules in a population) in an aqueous medium (e.g., water) at pH 7. The electric charge may be negative (anionic) or positive (cationic). If more than one electric charge is present, the electric charges may be negative (anionic) and/or positive (cationic), for example, both a negative and a positive charge may be present (zwitterionic).

Examples of ionic polymers include, without limitation, ionic polysaccharides, such as hyaluronic acid, chondroitin sulfate, alginic acid, xanthan gum, chitosan and N-alkyl chitosan derivatives.

According to another aspect of embodiments described herein, there is provided a method of reducing a friction coefficient of a surface, the method comprising contacting the surface with liposomes according to any of the respective embodiments described herein. In some embodiments, the method is effected by contacting the surface with a composition comprising the liposomes and a carrier (optionally a lubricant composition according to any of the respective embodiments described herein).

In some of any one of the embodiments described herein which relate to a lubrication, according to any one of the aspects described herein, the lubrication is optionally effected according to any of the embodiments described in International Patent Application PCT/IL2015/050605 (published as WO 2015/193887) and/or PCT/IL2015/050606 (published as WO 2015/193888).

In some embodiments, the method further comprises contacting the surface with a water-soluble polymer (e.g., according to any of the respective embodiments described herein), optionally prior to and/or concomitantly with contacting the surface with liposomes. In some embodiments, the method is effected by contacting the surface with a composition comprising the liposomes and the water-soluble polymer (optionally a lubricant composition comprising the water-soluble polymer according to any of the respective embodiments described herein), optionally in combination with an aqueous liquid.

In some of any one of the embodiments described herein which relate to a method and/or lubrication composition for reducing a friction coefficient of a surface, the surface is a hydrogel surface. In some embodiments, the hydrogel consists essentially of a polymer and an aqueous liquid (optionally water).

In some of any one of the embodiments described herein which relate to a method and/or lubrication composition for reducing a friction coefficient of a surface, the surface is a contact lens surface.

In some of any one of the embodiments described herein which relate to a contact lens, according to any one of the aspects described herein, the contact lens comprises a hydrogel surface. In some embodiments, the contact lens comprises a hydrogel surface and a rigid center. In some embodiments, the contact lens consists essentially of a hydrogel.

The hydrogel may comprise any material known in the art for use in contact lens hydrogels. Examples of such hydrogel materials include, without limitation, alphafilcon A, asmofilcon A, balafilcon A, bufilcon A, comfilcon A, crofilcon, deltafilcon A, dimefilcon, droxifilcon A, enfilcon A, ctafilcon A, galyfilcon A, hefilcon A, hefilcon B, hilafilcon A, hilafilcon B, hioxifilcon A, hioxifilcon D, isofilcon, lidofilcon A, lidofilcon B, lotrafilcon B, mafilcon, methafilcon A, methafilcon B, narafilcon A, narafilcon B, ocufilcon A, ocufilcon B, ofilcon A, omafilcon A, perfilcon, phemfilcon A, polymacon, scafilcon A, senofilcon A, surfilcon, tefilcon, tetrafilcon A, tetrafilcon B, vifilcon A, and xylofilcon A.

In some embodiments of any one of the embodiments described herein, the hydrogel comprises a polymer consisting of poly(2-hydroxyethyl methacrylate) and/or a silicone. In some embodiments, the polymer comprises a silicone. Such polymers may optionally comprise small amounts of additional monomers (e.g., cross-linking monomers) copolymerized with the 2-hydroxyethyl methacrylate or silicone monomer. For example, 2-hydroxyethyl methacrylate may optionally be copolymerized with vinyl pyrrolidone, methyl methacrylate, methacrylic acid (an anionic monomer), ethylene glycol dimethacrylate (a cross-linking monomer) and/or 3-(ethyldimethyl-ammonium)propyl methacrylamide (a cationic monomer) in a contact lens hydrogel.

### Physiological Surfaces:

In some embodiments of any of the embodiments described herein relating to a method and/or lubrication composition for reducing a friction coefficient of a surface, the surface is a physiological surface, and a carrier used with the liposomes (e.g., in a lubricant (e.g., sterile) composition according to any of the respective embodiments described herein) is a physiologically acceptable carrier.

In some embodiments, a surface for which a friction coefficient is reduced according any of the respective embodiments described herein is an articular surface of a synovial joint.

In some embodiments, the method of reducing a friction coefficient of a surface is for use in the treatment of a synovial joint disorder associated with an increased friction coefficient of an articular surface in the synovial joint.

In some embodiments of any of the embodiments described herein relating to a liposome, the liposome is for use in the treatment of a synovial joint disorder associated with an increased friction coefficient of an articular surface in the synovial joint.

According to another aspect of embodiments described herein, there is provided a use of a liposome according to any of the respective embodiments described herein in the manufacture of a medicament for treating a synovial joint disorder associated with an increased friction coefficient of an articular surface in the synovial joint.

Examples of synovial joint disorders associated with an increased friction coefficient of an articular surface, and treatable according to embodiments of various aspects of the invention, include, without limitation, arthritis, traumatic joint injury, locked joint (also known in the art as joint locking), and joint injury associated with surgery.

In some embodiments, the arthritis is osteoarthritis, rheumatoid arthritis and/or psoriatic arthritis.

In some embodiments, the locked joint is associated with osteochondritis dissecans and/or synovial osteochondromatosis.

The joint injury associated with surgery described herein may optionally be associated with surgery which directly inflicts damage on an articular surface (e.g., by incision), and/or surgery which damages an articular surface only indirectly. For example, surgery which repairs or otherwise affects tissue in the vicinity of the joint (e.g., ligaments and/or menisci) may be associated with joint injury due to altered mechanics in the joint.

The traumatic joint injury described herein may optionally be injury caused directly by trauma (e.g., inflicted at the time of the trauma) and/or injury caused by previous trauma (e.g., a post-traumatic injury which develops sometime after the trauma).

In some of any of the respective embodiments, the (e.g., sterile) composition according to any of the respective embodiments described herein and in any combination thereof, is for use in the treatment of a synovial joint disorder, for example, wherein the treatment comprises intra-articular administration of the (e.g., sterile) composition.

According to an aspect of some embodiments of the invention, there is provided a (e.g., sterile) composition according to any of the respective embodiments described herein for use in the manufacture of a medicament for the treatment of a synovial joint disorder, for example, wherein the treatment comprises intra-articular administration of the (e.g., sterile) composition.

According to an aspect of some embodiments of the invention, there is provided a method of treating a synovial joint disorder in a subject in need thereof, the method comprising administering to the subject a sterile composition according to any of the respective embodiments described herein, for example, via articular administration.

Examples of synovial joint disorders treatable according to embodiments of various aspects of the invention, include, without limitation, arthritis (e.g., osteoarthritis, rheumatoid arthritis and/or psoriatic arthritis), bursitis, carpal tunnel syndrome, fibromyositis, gout, locked joint (optionally associated with osteochondritis dissecans and/or synovial osteochondromatosis), tendinitis, traumatic joint injury (optionally caused directly by trauma, e.g., inflicted at the time of the trauma, and/or by previous trauma, e.g., a post-traumatic injury which develops sometime after the trauma), and joint injury associated with surgery (optionally associated with surgery which directly inflicts damage on an articular surface, e.g., by incision, and/or surgery which damages an articular surface only indirectly; for example, surgery which repairs or otherwise affects tissue in the vicinity of the joint, such as ligaments and/or menisci, may be associated with joint injury due to altered mechanics in the joint). Osteoarthritis is an exemplary synovial joint disorder treatable according to some embodiments of the invention.

In some of any of the respective embodiments, treatment of a synovial joint disorder (e.g., osteoarthritis) is characterized by a reduction in pain, for example, upon movement, at night, and/or at rest.

In such embodiments, reduction in pain may optionally be determined by any suitable technique known in the art. Examples of suitable techniques for determining reduction in pain include, without limitation, Brief Pain Inventory (e.g., short form), physical activity test (e.g., timed up and go), VAS (Visual Analogue Scale) questionnaire for assessing pain (no pain to unbearable pain), WOMAC (Western Ontario and McMaster Universities) scale, and/or KOOS (Knee Injury and Osteoarthritis Outcome Score).

In some of any of the respective embodiments, treatment of a synovial joint disorder (e.g., osteoarthritis) is characterized by an improvement in articular physiology.

In some of any of the respective embodiments, improvement in articular physiology is determined by a Kellgren-Lawrence scale of radiological severity, e.g., wherein the improvement is characterized as a reduction in severity. In some such embodiments, the treatment is further characterized by a reduction in pain (e.g., according to any of the respective embodiments described herein).

In some of any of the respective embodiments, improvement in articular physiology is determined by range of motion of an afflicted joint (e.g., wherein the improvement is characterized as an increase in range of motion of the joint), optionally in addition to being characterized as a reduction in severity according to a Kellgren-Lawrence scale. In some such embodiments, the treatment is further characterized by a reduction in pain (e.g., according to any of the respective embodiments described herein).

In some of any of the respective embodiments, improvement in articular physiology is characterized by an increase in physical activity (e.g., activity involving the afflicted joint), optionally in addition to being characterized as a reduction in severity according to a Kellgren-Lawrence scale and/or as an increase in range of motion (e.g., according to any of the respective embodiments described herein). In some such embodiments, the treatment is further characterized by a reduction in pain (e.g., according to any of the respective embodiments described herein).

In some of any of the respective embodiments, improvement in articular physiology is characterized by an increase in quality of life, optionally in addition to being characterized as a reduction in severity according to a Kellgren-Lawrence scale, an increase in range of motion and/or an increase in physical activity (e.g., according to any of the respective embodiments described herein). In some such embodiments, the treatment is further characterized by a reduction in pain (e.g., according to any of the respective embodiments described herein).

In some of any of the respective embodiments, improvement in articular physiology is determined by at least one, or at least two, or at least three or all four of a Kellgren-Lawrence scale of radiological severity, range of motion of joint, physical activity, and quality of life (according to any of the respective embodiments described herein). In some such embodiments, the treatment is further characterized by a reduction in pain (e.g., according to any of the respective embodiments described herein).

A composition for use in treating a synovial joint disorder (e.g., osteoarthritis) according to any of the respective embodiments described herein may optionally comprise one or more therapeutically active agent (e.g., according to any of the respective embodiments described herein). Examples of therapeutically active agents suitable for a composition for treating a synovial joint disorder include, without limitation, analgesics and anti-inflammatory agents.

Examples of suitable analgesics include, without limitation, allylprodine, alphamethylfentanyl, AP-237, bezitramide, butorphanol, buprenorphine, carfentanyl, clonidine, codeine, desmethylprodine, dextromoramide, dexocine, difenoxin, dihydrocodeine, dihydroctorphine, dihydromorphine, diphenoxylate, dipipanone, eluxadoline, ethylmorphine, etorphine, fentanyl, heterocodeine, hydrocone, hydromorphone, ketamine, ketobemidone, lefetamine, levomethadyl (e.g., levomethadyl acetate), levomethorphan, levorphanol, loperamide, meptazinol, methadone, mexiletine, mitragynine, morphine, nalbuphine, ohmefentanyl, oxycodone, oxymorphone, paracetamol, pentazocine, pethidine, phenethylphenylacetoxypiperidine, piritramide, prodine, promedol, propoxyphene, remifentanil, sulfentanil, tapentadol, tilidine, and tramadol.

Non-steroidal anti-inflammatory agents (e.g., a non-steroidal anti-inflammatory agent described herein), as well as steroidal anti-inflammatory agents, may also be used as an analgesic.

Examples of suitable anti-inflammatory agents include, without limitation, alclofenac; alclometasone (e.g., alclometasone dipropionate); algestone (e.g., algestone acetonide); alpha amylase; amcinafal; amcinafide; amfenac (e.g., amfenac sodium); amiprilose (e.g., amiprilose hydrochloride); anakinra; anirolac; anitrazafen; apazone; aspirin; balsalazide disodium; bendazac; benoxaprofen; benzydamine (e.g., benzydamine hydrochloride); bromelains; broperamole; budesonide; carprofen; cicloprofen; cintazone; cliprofen; clobetasol (e.g., clobetasol propionate, clobetasone butyrate); clopirac; cloticasone (cloticasome propionate); cormethasone (cormethasone acetate); cortodoxone; deflazacort; desonide; desoximetasone; dexamethasone (e.g., dexamethasone dipropionate); diclofenac (e.g., diclofenac potassium, diclofenac sodium); diflorasone (e.g., diflorasone diacetate); diflumidone (e.g., diflumidone sodium); diflunisal; difluprednate; diftalone; drocinonide; endrysone; enlimomab; enolicam (e.g., enolicam sodium); epirizole; etodolac; etofenamate; felbinac; fenamole; fenbufen; fenclofenac; fenclorac; fendosal; fenpipalone; fentiazac; flazalone; fluazacort; flufenamic acid; flumizole; flunisolide (e.g., flunisolide acetate); flunixin (e.g., flunixin meglumine); fluocortin (e.g., fluorcortin butyl); fluorometholone (e.g., fluorometholone acetate); fluquazone; flurbiprofen; fluretofen; fluticasone (e.g., fluticasone propionate); furaprofen; furobufen; halcinonide; halobetasol (e.g., halobetasol propionate); halopredone (e.g., halopredone acetate); ibufenac; ibuprofen (e.g., ibuprofen aluminum, ibuprofen piconol); ilonidap; indomethacin (e.g., indomethacin sodium); indoprofen; indoxole; intrazole; isoflupredone (e.g., isoflupredone acetate); isoxepac; isoxicam; ketoprofen; lofemizole (e.g., lofemizole hydrochloride); lomoxicam; loteprednol (e.g., loteprednol etabonate); meclofenamate (e.g., meclofenamate sodium, meclofenamic acid); meclorisone (e.g., meclorisone dibutyrate); mefenamic acid; mesalamine; meseclazone; methylprednisolone (e.g., methylprednisolone suleptanate); momiflumate; nabumetone; naproxen (e.g., naproxen sodium); naproxol; nimazone; olsalazine (e.g., olsalazine sodium); orgotein; orpanoxin; oxaprozin; oxyphenbutazone; paranyline (e.g., paranyline hydrochloride); pentosan polysulfate (e.g., pentosan polysulfate sodium); phenbutazone (e.g., phenbutazone sodium glycerate); pirfenidone; piroxicam (e.g., piroxicam cinnamate, piroxicam olamine); pirprofen; prednazate; prifelone; prodolic acid; proquazone; proxazole (e.g., proxazole citrate); rimexolone; romazarit; salcolex; salicylate (e.g., salicylic acid); salnacedin; salsalate; sanguinarium (e.g., sanguinarium chloride); seclazone; sermetacin; sudoxicam; sulindac; suprofen; talmetacin; talniflumate; talosalate; tebufelone; tenidap (e.g., tenidap sodium); tenoxicam; tesicam; tesimide; tetrydamine; tiopinac; tixocortol (e.g., tixocortol pivalate); tolmetin (e.g., tolmetin sodium); triclonide; triflumidate; zidometacin; and zomepirac (e.g., zomepirac sodium).

Alternatively, or in addition, a liposome composition as described herein in any of the respective embodiments is co-administered to the subject with a therapeutically active agent as described herein.

The liposomes (and optionally also a water-soluble polymer described herein) may optionally be administered as part of a (e.g., sterile) composition (e.g., solution) that comprises a physiologically acceptable carrier, for example an aqueous carrier which is a physiologically acceptable carrier.

Herein throughout, the term “physiologically acceptable carrier” refers to a carrier or a diluent that does not cause significant irritation to a subject upon administration in the intended manner, and does not abrogate the activity and properties of (e.g., sterile) composition (e.g., the ability of liposomes therein to treat a condition and/or to reduce a friction coefficient of a surface, as described herein in any one of the respective embodiments). Examples, without limitations, of carriers are: propylene glycol, saline, emulsions and mixtures of organic solvents with water (or saline), as well as solid (e.g., powdered) and gaseous carriers.

Techniques for formulation and administration of compounds (e.g., liposomes) may be found in “Remington's Pharmaceutical Sciences” Mack Publishing Co., Easton, PA, latest edition, which is incorporated herein by reference.

The (e.g., sterile) compositions (e.g., solutions) according to any one of the embodiments of the present invention may be manufactured by processes well known in the art, e.g., by means of conventional mixing or dissolving processes.

The (e.g., sterile) compositions (e.g., solutions) for use in accordance with the present invention thus may be formulated in conventional manner using one or more physiologically acceptable carriers, which facilitate processing of the liposomes (and optionally also a water-soluble polymer described herein) into preparations which can be used pharmaceutically. Proper formulation is dependent upon the route of administration chosen.

For injection, the (e.g., sterile) composition according to any of the respective embodiments described herein or the liposomes described herein (optionally with a water-soluble polymer described herein) may be formulated in aqueous solutions using a suitable aqueous carrier, preferably a physiologically compatible buffers such as Hank's solution, Ringer's solution, histidine buffer, or physiological saline buffer with or without organic solvents such as propylene glycol, polyethylene glycol.

The (e.g., sterile) composition according to any of the respective embodiments described herein or the liposomes described herein (optionally with a water-soluble polymer described herein) may be formulated for parenteral administration, e.g., by bolus injection or continuous infusion. Formulations for injection may be presented in unit dosage form, e.g., in ampoules or in multi-dose containers with optionally, an added preservative. The (e.g., sterile) compositions may be suspensions, solutions or emulsions in oily or aqueous vehicles, and may contain formulatory agents such as suspending, stabilizing and/or dispersing agents.

The (e.g., sterile) composition according to any of the respective embodiments described herein or liposomes described herein (optionally with a water-soluble polymer described herein) may be formulated as an aqueous solution per se. Additionally, the (e.g., sterile) composition (e.g., solution) may be in the form of a suspension and/or emulsions (e.g., the aqueous phase of a suspension or water-in-oil, oil-in-water or water-in-oil-in-oil emulsion), for example, in order to increase the viscosity of the formulation. Aqueous injection suspensions may contain substances, which increase the viscosity of the suspension, such as sodium carboxymethyl cellulose, sorbitol or dextran. Optionally, the suspension may also contain suitable stabilizers or agents, which increase the solubility of the liposomes described herein (and/or the optional water-soluble polymer described herein), for example, to allow for the preparation of highly concentrated solutions.

In some embodiments, the liposomes described herein (optionally with a water-soluble polymer described herein) may be in powder form for constitution with a suitable vehicle, e.g., sterile, pyrogen-free water, before use.

The (e.g., sterile) composition or solutions according to any of the respective embodiments described herein may be formulated wherein the liposomes are contained in an amount effective to achieve the intended purpose, for example, an amount effective to prevent, alleviate or ameliorate symptoms of a disorder in the subject being treated. Additionally or alternatively, the (e.g., sterile) composition may be in the form of a suspension and/or emulsions (e.g., water-in-oil, oil-in-water or water-in-oil-in-oil emulsion), for example, in order to increase the viscosity of the formulation.

Aqueous injection suspensions may contain substances, which increase the viscosity of the suspension, such as sodium carboxymethyl cellulose, sorbitol or dextran. Optionally, the suspension may also contain suitable stabilizers or agents, which increase the solubility and/or stability of the liposomes described herein, for example, to allow for the preparation of highly concentrated solutions.

The dosage may vary depending upon the dosage form employed, the route of administration utilized, the location of administration (e.g., the volume and/or surface of the region contacted with the liposomes), the judgment of the prescribing physician, etc.

The amount of a composition to be administered will, of course, be dependent on the subject being treated, the severity of the affliction, the manner of administration, the judgment of the prescribing physician, etc.

The (e.g., sterile) compositions (e.g., solutions) according to embodiments of the present invention may, if desired, be presented in a pack or dispenser device, such as an FDA (the U.S. Food and Drug Administration) approved kit, which may contain one or more unit dosage forms containing the active ingredient(s)(e.g., liposomes described herein). The pack may, for example, comprise metal or plastic foil, such as, but not limited to a blister pack. The pack or dispenser device may be accompanied by instructions for administration. The pack or dispenser may also be accompanied by a notice associated with the container in a form prescribed by a governmental agency regulating the manufacture, use or sale of pharmaceuticals, which notice is reflective of approval by the agency of the form of the (e.g., sterile) compositions for human or veterinary administration. Such notice, for example, may be of labeling approved by the U.S. Food and Drug Administration for prescription drugs or of an approved product insert. Compositions (e.g., sterile) comprising liposomes (optionally with a water-soluble polymer described herein), as described herein in any one of the respective embodiments, formulated in a physiologically acceptable carrier may also be prepared, placed in an appropriate container, and labeled for treatment of an indicated condition or diagnosis, as is detailed herein.

### Inhibition of Biofilm Formation and Biofouling:

Liposomes and bilayers described herein may optionally be useful for inhibiting adhesion, biofouling and/or biofilm formation on a surface, for example, a surface coated by a bilayer described herein, and/or contacted with liposomes described herein.

According to an aspect of some embodiments of the invention, there is provided a method of inhibiting adsorption of a biofouling-promoting agent on a surface of a substrate. The method, according to some embodiments of the present invention, is effected by contacting the substrate with a composition which comprises liposomes according to any of the respective embodiments described herein.

The term “biofouling-promoting agent”, as used herein throughout, refers to an agent whose presence facilitates formation and/or participates in formation of a biofilm (as defined herein) on a substrate surface. An agent is considered to facilitate formation of a biofilm on a substrate surface when a presence of the agent enhances formation of a biofilm on a substrate surface as compared to formation of a biofilm on the same substrate surface in an absence of the agent. An agent is considered to participate in formation of a biofilm on a substrate surface when the biofilm formed on the surface comprises the agent as a portion of the biofilm.

In some embodiments of any of the embodiments described herein, an agent is identified as a biofouling-promoting agent by comparing growth (e.g., over the course of 1, 2, 3, 4, 5, 6 or 7 days) of a biofilm (e.g., P. aeruginosa) on the surface in the presence of an aqueous liquid (e.g., water or broth, optionally at 37° C.) and the agent, to growth of a biofilm (under the same conditions) on the surface in the presence of the same aqueous liquid (e.g., water or broth) without the agent. The agent is optionally mixed within the aqueous liquid, or alternatively, adsorbed onto the surface prior to exposure of the surface to the aqueous liquid. The growth of the biofilm is considered as the biofilm load at the end of the growth period (e.g., 1, 2, 3, 4, 5, 6 or 7 days) minus the initial biofilm load. Optionally, the measurement is performed such that the initial biofilm is substantially zero (e.g., absent or at least undetectable), for example, the microorganism is in a planktonic form, such that growth of the biofilm is considered as the biofilm load at the end of the growth period. In some embodiments of any of the embodiments described herein, biofilm load is defined as an area of the biofilm.

In some embodiments of any of the embodiments described herein, biofilm load is defined as a mass and/or volume of the biofilm.

In some embodiments of any of the embodiments described herein, biofilm load is defined as a number of cells in the biofilm.

The biofilm load may optionally be determined using any technique known in the art for detecting and quantifying an amount of cells and/or microorganisms in a biofilm.

In some of these embodiments, an agent is considered a biofouling-promoting agent if the growth of a biofilm in its presence is at least 10% higher than growth of a biofilm in the absence of the agent.

In some of these embodiments, an agent is considered a biofouling-promoting agent if the growth of a biofilm in its presence is at least 20% higher than growth of a biofilm in the absence of the agent.

In some of these embodiments, an agent is considered a biofouling-promoting agent if the growth of a biofilm in its presence is at least 50% higher than growth of a biofilm in the absence of the agent.

In some of these embodiments, an agent is considered a biofouling-promoting agent if the growth of a biofilm in its presence is at least 100% higher than (i.e., two-fold) the growth of a biofilm in the absence of the agent.

Examples of biofouling-promoting agents include, without limitation, a biofouling-promoting protein and a biofouling-promoting polysaccharide, that is, any protein or polysaccharide which is a biofouling-promoting agent as defined herein.

In some embodiments of any of the embodiments described herein, the biofouling-promoting agent is a protein.

In some embodiments of any of the embodiments described herein, the method is considered as being capable of inhibiting adsorption of a biofouling-promoting agent when the method is capable of inhibiting adsorption of a selected biofouling-promoting agent (e.g., the selected agent is considered representative of biofouling-promoting agents in general). In some embodiments, the selected biofouling-promoting agent is a protein. In some embodiments, the selected protein is an antibody which does not exhibit any specific affinity to the substrate (e.g., an anti-IgG antibody, as exemplified herein).

The term “biofilm”, as used herein throughout, refers to an aggregate of living cells which are stuck to each other and/or immobilized onto a surface as colonies. The cells are frequently embedded within a self-secreted matrix of extracellular polymeric substance (EPS), also referred to as “slime”, which is a polymeric sticky mixture of nucleic acids, proteins and polysaccharides.

In the context of the present embodiments, the living cells forming a biofilm can be cells of a unicellular microorganism, including prokaryotes (e.g., bacteria, archacal microorganisms) and eukaryotes such as fungi and protists (e.g., algae, euglena, protozoa, dinoflagellates, apicomplexa, trypanosomes, amoebae) and the like; or cells of multicellular organisms, in which case the biofilm can be regarded as a colony of cells (as in the case of the unicellular organisms) or as a lower form of a tissue.

According to some embodiments of any of the embodiments of the present invention, the cells are of microorganism origins, and the biofilm is a biofilm of microorganisms, such as bacteria, archacal microorganisms, protists and fungi. The cells of a microorganism growing in a biofilm are typically physiologically distinct from cells in the “planktonic” form of the same organism, which by contrast, are single cells that may float or swim in a liquid medium.

The substrate may be any substrate described herein, and encompasses any surface, structure, product or material which can support, harbor or promote the growth of a microorganism. The substrate is optionally a portion of an object (e.g., an article of manufacture) which can support, harbor or promote the growth of a microorganism. Such a portion of an object may span only a portion of an area of the object, such that a surface of the substrate represents only a portion of a surface of the object (e.g., a portion most likely to support, harbor or promote the growth of a microorganism); and/or span only a portion of the thickness of the object (e.g., along an axis perpendicular to a surface of the substrate and object), such that the substrate does not include the entire volume of the object lying underneath a surface of the substrate (which may represent the entire surface of the object or only a portion of the surface of the object). Non-limiting examples include the inner walls of a storage container (e.g., a box, a can) and/or conduit (e.g., a tubes, a pipe) for an organic product susceptible to spoilage associated with biofouling, for example, food and/or drink (e.g., a food container, a water pipe), surfaces intended to come into contact with such an organic product (e.g., agricultural and/or food processing machinery, a kitchen surface, water-purification equipment), and surfaces exposed to moisture (e.g., bathroom walls, water system components, outer surfaces of housing exposed to rain, surfaces in the vicinity of water leakage).

In some embodiments, the substrate is a medical device or any other device which is intended for contacting a living tissue, as defined herein.

In some embodiments of any of the embodiments described herein, the inhibition of adsorption described herein is for reducing adhesion of pathogenic microorganisms (e.g., any biofilm-forming microorganism described herein which is potentially pathogenic) to a medical device (e.g., any medical device described herein).

In some embodiments of any of the embodiments described herein, adsorption of the biofouling-promoting agent (any biofouling-promoting agent described herein) on the surface of the substrate subjected to a method described herein (according to any of the respective embodiments) is reduced by at least 10% relative to adsorption on the surface of the substrate in the absence of the composition which comprises liposomes In some embodiments, adsorption is reduced by at least 20%. In some embodiments, adsorption is reduced by at least 30%. In some embodiments, adsorption is reduced by at least 40%. In some embodiments, adsorption is reduced by at least 50%. In some embodiments, adsorption is reduced by at least 60%. In some embodiments, adsorption is reduced by at least 70%. In some embodiments, adsorption is reduced by at least 80%. In some embodiments, adsorption is reduced by at least 90%.

Reduction of an amount of adsorbed biofouling-promoting agent may optionally be determined using any technique known in the art for detecting and quantifying an amount of agent, including, without limitation, using a labeled biofouling-promoting agent (e.g., as exemplified herein in the Examples section). The reduction is optionally measured by contacting each of the aforementioned surfaces (e.g., for 2 hours) with an aqueous solution (optionally comprising phosphate buffer, e.g., 0.1 M phosphate) of the biofouling-promoting agent (e.g., at 37° C. and/or pH 7), followed by repeated rinses to remove non-adsorbed agent (e.g., as exemplified in the examples section herein). The concentration of the biofouling-promoting agent in the aqueous solution is optionally 1 μg/mL or the concentration of a saturated solution of the agent, whichever concentration is lower.

Herein throughout, the term “biofilm-promoting conditions” refers to conditions suitable for formation and growth of a biofilm of a cell (e.g., P. aeruginosa), for example, wherein a surface is in contact (e.g., over the course of 1, 2, 3, 4, 5, 6 or 7 days) with an aqueous liquid (e.g., water or broth, optionally at 37° C.) containing such cells.

In some embodiments of any of the embodiments described herein, biofilm load is defined as an area of the biofilm.

In some embodiments of any of the embodiments described herein, biofilm load is defined as a mass and/or volume of the biofilm.

In some embodiments of any of the embodiments described herein, biofilm load is defined as a number of cells in the biofilm.

The biofilm load may optionally be determined using any technique known in the art for detecting and quantifying an amount of cells and/or microorganisms in a biofilm.

In some embodiments of any of the embodiments described herein, the time period of biofilm formation, after which biofilm load is determined, is in determined in accordance with the biofilm load, for example, the time period being a time period after which a biofilm covers 100%, 50%, or any other pre-determined percentage of the area of the substrate in the absence of inhibition of biofilm formation by contact with a composition comprising liposomes. For example, if a biofilm grows to cover 50% of a surface in the absence of biofilm formation inhibition, and during the same a time period, a biofilm grows to cover 30% of a surface in the presence of biofilm formation inhibition, then inhibition of biofilm formation may be considered to result in a reduction of 40% (i.e., (50%-30%)/50%) in biofilm formation.

Herein, the phrase “upon contact with the agent” means that in addition to the biofilm-promoting conditions, the agent is also present (e.g., in the aqueous liquid containing the cells).

According to an aspect of some embodiments of the invention, there is provided a method of inhibiting biofilm formation on a surface of a substrate (as defined herein in any embodiment and any combination of embodiments), the method comprising contacting (as described in any of the respective embodiments described herein) the substrate with a composition which comprises liposomes.

In some embodiments, “inhibiting biofilm formation” refers to the prevention of formation of a biofilm; and/or to a reduction in the rate of buildup of a biofilm; and/or to a reduction in the mass of a biofilm, the area or the volume of the biofilm, or in the number of cells forming the biofilm.

In some embodiments of any of the embodiments described herein, the inhibition of adsorption described herein is for reducing adhesion of pathogenic microorganisms (e.g., any biofilm-forming microorganism described herein which is potentially pathogenic) to a medical device. Such a reduction may result in inhibiting biofilm formation, as defined in some embodiments herein.

In some embodiments of any of the embodiments described herein, biofilm formation on the surface of the substrate subjected to a method described herein (according to any of the respective embodiments) is reduced by at least 10% relative to biofilm formation on the surface of the substrate in the absence of the composition which comprises liposomes. In some embodiments, biofilm formation is reduced by at least 20%. In some embodiments, biofilm formation is reduced by at least 30%. In some embodiments, biofilm formation is reduced by at least 40%. In some embodiments, biofilm formation is reduced by at least 50%. In some embodiments, biofilm formation is reduced by at least 60%. In some embodiments, biofilm formation is reduced by at least 70%. In some embodiments, biofilm formation is reduced by at least 80%. In some embodiments, biofilm formation is reduced by at least 90%.

The reduction in biofilm formation is optionally determined by measuring a biofilm load (in accordance with any of the respective embodiments described herein) for a biofilm of a cell (e.g., P. aeruginosa) on each surface after being subjected to biofouling-promoting conditions, as defined herein (e.g., over the course of 1, 2, 3, 4, 5, 6 or 7 days, or any other time period as described herein).

Any of the embodiments described herein relating to inhibition of biofilm formation and/or biofouling may optionally be effected by a composition which is essentially the same as a lubricant composition according to any of the respective embodiments described herein (although optionally identified for inhibition of biofilm formation and/or biofouling rather than for lubrication).

In some of any one of the embodiments described herein which relate to a inhibition of adhesion, biofilm formation and/or biofouling, according to any one of the aspects described herein, the inhibition is optionally effected according to any of the embodiments described in Israel Patent Application No. 234929 and/or International Patent Application PCT/IL2015/050987 (published as WO 2016/051413).

### Additional Compositions and Uses:

In view of their optional sterile nature, compositions according to the respective embodiments described herein may be useful for use in a physiological environment, such as internal physiological environment or an ophthalmic environment. Accordingly, the aqueous carrier according to any of the respective embodiments described herein may optionally be selected in accordance with an intended use, for example, a physiologically acceptable carrier, as described herein, and/or an ophthalmically acceptable carrier.

A use in a physiological environment may optionally be for reducing a friction coefficient (also referred to herein as “lubrication”, “lubricating” and variants thereof) of a physiological surface (e.g., an articular surface or a surface of an eye) and/or non-physiological surface (e.g., contact lens surface), for example, in treating a disease or disorder associated with an increased friction coefficient of the surface. Reduction of a friction coefficient of a surface may optionally be effected by any one or more compound present in liposomes (according to any of the respective embodiments described herein), including a bilayer-forming lipid and/or polymeric compound according to any of the respective embodiments described herein.

In some of any of the embodiments described herein, a (e.g., sterile) composition comprising the liposomes as described herein is for rinsing, cleaning and/or immersing therein a contact lens. In some such embodiments, the composition comprises an ophthalmically acceptable carrier as described herein in any of the respective embodiments, and may optionally be allowed to remain on the contact lens following rinsing, cleaning and/or immersing in the solution, as the residual solution will not harm the eye when the contact lens is placed on the eye. In some alternative embodiments, the aqueous carrier is not an ophthalmically acceptable carrier (e.g., wherein the carrier comprises a preservative and/or a concentration of preservative which is not ophthalmically acceptable), and the (e.g., sterile) composition (e.g., a composition having a contact lens immersed therein according to any of the respective embodiments described herein) may optionally be for immersing a contact lens for an extended period of time (e.g., when the contact lens is not in use, for example, at night) and/or for storage for an extended period of time (e.g., between contact lens manufacture and initial use), while limiting the risk of bacterial growth in the solution; for example, wherein such a composition is rinsed with an ophthalmically acceptable liquid (e.g., water, saline) solution prior to placing the contact lens on the eye.

In some of any of the embodiments described herein relating to a contact lens, the (e.g., sterile) composition is for immersing therein a contact lens (e.g., to maintain moisture of a contact lens, optionally while also reducing a friction coefficient of the contact lens). In some such embodiments, the carrier of the (e.g., sterile) composition comprises additional ingredients suitable for effecting cleaning, for example, a preservative. Such a composition may optionally be provided together with a contact lens immersed therein as a single product, e.g., wherein a sterile contact lens and sterile composition are packaged together. The sterile composition may optionally be intended to be rinsed off by a more ophthalmically acceptable composition prior to insertion into the eye.

In some of any of the embodiments described herein relating to a contact lens, the (e.g., sterile) composition is for rinsing a contact lens (e.g., to remove another composition from the contact lens, such a liquid which is not ophthalmically acceptable, and/or to reduce a friction coefficient of the contact lens). Such a composition may optionally be provided as a separate product from the contact lens. In some such embodiments, the carrier of the sterile composition is an ophthalmically acceptable carrier as described herein in any of the respective embodiments.

In some of any of the embodiments described herein relating to a contact lens, the (e.g., sterile) composition is for cleaning a used and/or new contact lens (e.g., to remove bacteria and/or other impurities, optionally while also reducing a friction coefficient of the contact lens). In some such embodiments, the carrier of the (e.g., sterile) composition comprises additional ingredients suitable for effecting cleaning, for example, an antimicrobial agent (e.g., a peroxide and/or other oxidizing agent) and/or a detergent for removing impurities is an ophthalmically acceptable carrier as described herein in any of the respective embodiments. Such a composition may optionally be provided as a separate product from the contact lens, which may optionally be intended to be rinsed off by a more ophthalmically acceptable composition prior to insertion into the eye.

In some of any of the respective embodiments, the (e.g., sterile) composition according to any of the respective embodiments described herein is for use in treating an ocular disease, for example, a dry eye syndrome and any other ocular disease.

Compositions (e.g., solutions) according to embodiments of the present invention may, if desired, be presented in a pack or dispenser device, such as an FDA (the U.S. Food and Drug Administration) approved kit, which may contain one or more unit dosage forms containing the active ingredient(s)(e.g., liposomes described herein). The pack may, for example, comprise metal or plastic foil, such as, but not limited to a blister pack. The pack or dispenser device may be accompanied by instructions for administration. The pack or dispenser may also be accompanied by a notice associated with the container in a form prescribed by a governmental agency regulating the manufacture, use or sale of pharmaceuticals, which notice is reflective of approval by the agency of the form of the compositions for human or veterinary administration. Such notice, for example, may be of labeling approved by the U.S. Food and Drug Administration for prescription drugs or of an approved product insert. Sterile compositions comprising liposomes, as described herein in any one of the respective embodiments, formulated in a physiologically acceptable carrier may also be prepared, placed in an appropriate container, and labeled for treatment of an indicated condition, as is detailed herein.

According to an aspect of some embodiments of the invention, there is provided a composition or formulation comprising at least one water-soluble polymer (as described herein in any one of the respective embodiments), liposomes (as described herein in any one of the respective embodiments), an ophthalmically acceptable carrier (e.g., aqueous carrier), as described herein in any of the respective embodiments, and optionally a saccharide (e.g., a polysaccharide).

Herein, the phrase “ophthalmically acceptable carrier” refers to a carrier or a diluent that does not cause significant irritation to a subject when contacted with an eye (e.g., cornea and/or sclera) of the subject, and does not abrogate the activity and properties of the composition (e.g., the ability of liposomes therein to reduce a friction coefficient of a surface of a contact lens and/or a surface of the eye).

In some of any of the embodiments described in this aspect, the composition or formulation as described herein is a liquid formulation, and is also referred to herein interchangeably as “solution”. It is to be noted that herein throughout, the term “solution” encompasses any liquid formulation in which the ingredients, namely, at least the water-soluble polymer and the liposomes/lipids are included within a liquid carrier, whereby each of the ingredients can be dissolved or dispersed within the carrier. The term “solution” as used herein therefore encompasses also “dispersion”. The term “liquid formulation” as used herein encompasses both a solution and a dispersion.

According to some the present embodiments of this aspect, the bilayer-forming lipid comprises a first and a second glycerophospholipids as described in further detail hereunder. In some such embodiments, the bilayer-forming lipid comprises at least two lipid materials: a first glycerophospholipid featuring Tm lower than 25° C. and a second glycerophospholipid featuring Tm higher than 40° C.

According to some of the present embodiments of this aspect, a weight ratio of the first and second glycerophospholipids is such that the bilayer-forming lipid features Tm in a range of from 25 to 40, or from 26 to 39, or from 26 to 33, or from 28 to 33,° C.

According to some of any of the embodiments described herein, the weight ratio of the first and second glycerophospholipids in the composition or formulation as described in this aspect is at least 2:1, for example, is 2:1, or 3:2, or 3:1, or 4:1, or 5:2, or 5:1, or 5:3 (first glycerophospholipid: second glycerophospholipid).

According to some of any of the embodiments described herein, the weight ratio of the first and second glycerophospholipids in the composition or formulation as described in this aspect is 3:1 (first glycerophospholipid: second glycerophospholipid).

According to some of any of the embodiments described herein, the weight ratio of the first and second glycerophospholipids in the composition or formulation as described in this aspect ranges from 4:1 to 1:4, or from 3:1 to 1:3, or from 3:1 to 1:1 (first glycerophospholipid: second glycerophospholipid), including any intermediate values and subranges therebetween.

According to some of any of the embodiments which relate to the composition or formulation as described in this aspect, at least 50% or at least 60%, or at least 70%, by weight, of the total weight of the bilayer-forming lipid, is the first glycerophospholipid as described herein.

According to some of any of the embodiments which relate to the composition or formulation as described in this aspect, less than 50% or less than 40%, or less than 30%, by weight, of the total weight of the bilayer-forming lipid, is the second glycerophospholipid as described herein.

According to some of any of the embodiments which relate to a composition or formulation as described in this aspect, an amount of the first glycerophospholipid as described herein ranges from 30% to 90%, or from 30% to 80%, or from 40% to 90%, or from 40% to 80%, or from 50% to 90%, or from 50% to 80%, or from 60% to 90%, or from 60% to 80%, by weight, of the total weight of the bilayer-forming lipid, including any intermediate values and subranges therebetween.

According to some of any of the embodiments which relate to the composition or formulation as described in this aspect, an amount of the second glycerophospholipid as described herein ranges from 5% to 50%, or from 10% to 50%, or from 5% to 40%, or from 10% to 40%, or from 5% to 30%, or from 10% to 30%, or from 20% to 50%, or from 20% to 40%, or from 20% to 30%, by weight, of the total weight of the bilayer-forming lipid, including any intermediate values and subranges therebetween.

According to some of any of the embodiments which relate to the composition or formulation as described in this aspect, an amount of the first glycerophospholipid as described herein ranges from 30% to 90%, or from 30% to 80%, or from 40% to 90%, or from 40% to 80%, or from 50% to 90%, or from 50% to 80%, or from 60% to 90%, or from 60% to 80%, by weight, of the total weight of the bilayer-forming lipid, including any intermediate values and subranges therebetween, and an amount of the second glycerophospholipid as described herein ranges from 5% to 50%, or from 10% to 50%, or from 5% to 40%, or from 10% to 40%, or from 5% to 30%, or from 10% to 30%, or from 20% to 50%, or from 20% to 40%, or from 20% to 30%, by weight, of the total weight of the bilayer-forming lipid, including any intermediate values and subranges therebetween.

In some of any of the embodiments which relate to the composition or formulation as described in this aspect, the bilayer-forming lipid comprises a first glycerophospholipid featuring Tm lower than 25° C. and a second glycerophospholipid featuring Tm higher than 40° C., wherein a weight ratio of the first and second glycerophospholipids is such that the bilayer-forming lipid features Tm in a range of from 25 to 40, or from 26 to 39, or from 26 to 33, or from 28 to 33,° C., including any intermediate values and subranges therebetween.

According to some of any of the embodiments which relate to the composition or formulation as described in this aspect, the bilayer-forming lipid consists of the first and second glycerophospholipids as described herein.

According to some of any of the embodiments which relate to the composition or formulation as described in this aspect, the first glycerophospholipid is DMPC as described herein, although other glycerophospholipids featuring Tm as indicated are contemplated.

According to some of any of the embodiments which relate to the composition as described in this aspect, the second glycerophospholipid is DPPC as described herein, although other glycerophospholipids featuring Tm as indicated are contemplated.

According to some of any of the embodiments which relate to the composition or formulation as described in this aspect, the bilayer forming lipid material comprises or consists of DMPC and DPPC.

According to some of any of the embodiments which relate to the composition or formulation as described in this aspect, the bilayer forming lipid material comprises DMPC in an amount that ranges from 30% to 90%, or from 30% to 80%, or from 40% to 90%, or from 40% to 80%, or from 50% to 90%, or from 50% to 80%, or from 60% to 90%, or from 60% to 80%, by weight, of the total weight of the bilayer-forming lipid, including any intermediate values and subranges therebetween.

According to some of any of the embodiments which relate to the composition or formulation as described in this aspect, the bilayer forming lipid material comprises DMPC in an amount that ranges from 50% to 90%, or from 50% to 80%, or from 60% to 90%, or from 60% to 80%, by weight, of the total weight of the bilayer-forming lipid, including any intermediate values and subranges therebetween.

According to some of any of the embodiments which relate to the composition or formulation as described in this aspect, the bilayer forming lipid comprises DPPC in an amount that ranges 5% to 50%, or from 10% to 50%, or from 5% to 40%, or from 10% to 40%, or from 5% to 30%, or from 10% to 30%, or from 20% to 50%, or from 20% to 40%, or from 20% to 30%, by weight, of the total weight of the bilayer-forming lipid, including any intermediate values and subranges therebetween.

According to some of any of the embodiments which relate to the composition or formulation as described in this aspect, the bilayer forming lipid material comprises DMPC in an amount that ranges from 50% to 90%, or from 50% to 80%, or from 60% to 90%, or from 60% to 80%, by weight, of the total weight of the bilayer-forming lipid, including any intermediate values and subranges therebetween, and DPPC in an amount that ranges 5% to 50%, or from 10% to 50%, or from 5% to 40%, or from 10% to 40%, or from 5% to 30%, or from 10% to 30%, or from 20% to 50%, or from 20% to 40%, or from 20% to 30%, by weight, of the total weight of the bilayer-forming lipid, including any intermediate values and subranges therebetween.

According to some of these embodiments, the bilayer-forming lipid consists of DPPC and DMPC.

According to some of any of the embodiments which relate to the composition or formulation as described in this aspect, the bilayer-forming lipid comprises or consists of DMPC and DPPC, and a weight ratio DMPC: DPPC ranges from 1:1 to 5:1, or from 1:1 to 4:1, or from 1:1 to 3:1, preferably from 2:1 to 4:1, or from 2:1 to 3:1, including any intermediate values and subranges therebetween.

According to some of any of the embodiments which relate to the composition or formulation as described in this aspect, the bilayer-forming lipid comprises or consists of DMPC and DPPC, an amount of DMPC ranges from 20% to 80%, or from 50% to 80% by weight of the total weight of the bilayer-forming lipid, and an amount of DMPC ranges from 20% to 80%, or from 20% to 50% by weight of the total weight of the bilayer-forming lipid, including any intermediate values and subranges therebetween.

According to some of any of the embodiments which relate to the composition or formulation as described in this aspect, the bilayer-forming lipid comprises or consists of DMPC and DPPC, an amount of DMPC is about 75%, by weight of the total weight of the bilayer-forming lipid, and an amount of DMPC is about 25% by weight of the total weight of the bilayer-forming lipid.

According to some of any of the embodiments which relate to the composition or formulation as described in this aspect, the bilayer-forming lipid comprises or consists of DMPC and DPPC, an amount of DMPC is about 80%, by weight of the total weight of the bilayer-forming lipid, and an amount of DMPC is about 20% by weight of the total weight of the bilayer-forming lipid.

According to some of any of the embodiments which relate to the composition or formulation as described in this aspect, the bilayer-forming lipid comprises or consists of DMPC and DPPC, an amount of DMPC is about 70%, by weight of the total weight of the bilayer-forming lipid, and an amount of DMPC is about 30% by weight of the total weight of the bilayer-forming lipid.

According to some of any of the embodiments which relate to the composition or formulation as described in this aspect, the bilayer-forming lipid comprises or consists of DMPC and DPPC, an amount of DMPC is about 65%, by weight of the total weight of the bilayer-forming lipid, and an amount of DMPC is about 35% by weight of the total weight of the bilayer-forming lipid.

According to some of any of the embodiments which relate to the composition or formulation as described in this aspect, the bilayer-forming lipid comprises or consists of DMPC and DPPC, an amount of DMPC is about 60%, by weight of the total weight of the bilayer-forming lipid, and an amount of DMPC is about 40% by weight of the total weight of the bilayer-forming lipid.

It is to be appreciated that phase transitions, e.g., melting points (Tm), of the lipid bilayers and liposomes comprising the composition or formulation as described in this aspect may be determined by the skilled person by selecting suitable fatty acyl groups for inclusion in the lipids, for example, by selecting relatively short and/or unsaturated fatty acyl groups (e.g., myristoyl) to obtain a relatively low melting point; and/or by selecting relatively long and/or saturated fatty acyl groups (e.g., palmitoyl and/or stearoyl) to obtain a relatively high melting point.

In some embodiments of any one of the embodiments described herein, the liposomes described in this aspect are characterized by a phase transition melting point is a range as indicated herein.

In some embodiments relating to ophthalmic compositions or formulations as described herein, a mean diameter of liposomes ranges from about 100 nm to about 200 nm, or from about 100 nm to about 180 nm, of from about 100 nm to about 160 nm, including any intermediate values and subranges therebetween.

In some embodiments of any one of the embodiments described herein, the liposomes described herein are characterized by a surface charge, which may be a positive surface charge or a negative surface charge.

As used herein, the phrase “surface charge” refers to an electric charge at or near a surface, such as an interface of a liposome with a solution. The phrase “surface charge” encompasses an electric charge associated with an electric potential at a surface (e.g., such that a positive electric potential at a surface is indicative of a positive surface charge, whereas a negative electric potential at a surface is indicative of a negative surface charge); as well as an electric charge which is closer to a surface than an electric charge of an opposite sign (e.g., as in a zwitterion wherein the positive charge is closer to the surface than the negative charge, or vice versa), such that an ion near the surface will interact primarily with the electric charge near the surface (due to the proximity) as opposed to the electric charge of an opposite sign. For example, phosphatidylcholine liposomes typically exhibit a positive surface charge because the positive charge of the choline group is closer to the liposome surface than the negative charge of the phosphate group.

Optionally, a surface charge of a liposome as described herein is associated with a net charge of the lipid molecules in the liposome, for example, a liposome comprising anionic lipids has a negative surface charge, and/or a liposome comprising cationic lipids has a positive surface charge.

Alternatively or additionally, a surface charge of a liposome as described herein is associated with a dipole of lipid molecules (e.g., zwitterionic lipid molecules) in the liposome, for example, a liposome comprising a zwitterionic lipid comprising a phosphocholine group may have a positive surface charge due to the positively charged ammonium groups in the phosphocholine groups being (on average) closer to the surface of the liposomes than the negatively charged phosphate groups in the phosphocholine groups.

The skilled person will be readily capable of determining a surface charge. For example, the sign of a surface charge may be determined by comparing the propensity of a surface (e.g., of a liposome) to bind to anionic vs. cationic compounds (e.g., labeling compounds). Alternatively, or in addition, surface charge can be determined by zeta potential measurements, using techniques well known in the art.

In some embodiments of any one of the embodiments described herein, the liposomes as described herein rupture upon contact with at least one water-soluble polymer as described herein in any of the respective embodiments (e.g., upon contact of the at least one water-soluble polymer on a surface of the liposome). Such liposome rupture may optionally result in a lipid bilayer in the liposomes being converted from a curved geometry (e.g., as in the relatively spherical liposomes) to a flatter geometry which complements the geometry of the surface and/or the water-soluble polymer(s) attached to the surface (e.g., thereby enhancing affinity of the lipids to the surface); and/or which results in a flatter, smoother lipid-coated surface (e.g., thereby further reducing friction).

In some embodiments of any one of the embodiments described herein, liposomes as described herein and water-soluble polymer(s) are selected such that the selected water-soluble polymer(s) is effective at rupturing the selected liposomes.

In some embodiments of any one of the embodiments described herein in this aspect relating to a water-soluble polymer comprising an ionic polymer, at least 75% of the ionic groups in the polymer have the same charge, that is, at least 75% of the ionic groups are cationic groups or are anionic groups, such that the polymer is substantially cationic or anionic, respectively. In some embodiments, at least 90% of the ionic groups in the polymer have the same charge. In some embodiments, at least 95% of the ionic groups in the polymer have the same charge. In some embodiments, at least 98% of the ionic groups in the polymer have the same charge. In some embodiments, at least 99% of the ionic groups in the polymer have the same charge.

In some embodiments of any one of the embodiments described herein in this aspect, about 50% of the ionic groups in the polymer have a positive charge and about 50% of the ionic groups in the polymer have a negative charge, such that the polymer is substantially zwitterionic.

In some embodiments of any one of the embodiments described herein in this aspect, the ionic polymer is characterized by a charge density of from 1 to 6 charged groups (ionic groups) per 1 kDa molecular weight of the polymer. In some embodiments, the ionic polymer has from 1.5 to 4 charged groups per 1 kDa. In some embodiments, the ionic polymer has from 2 to 3 charged groups per 1 kDa.

In some embodiments of any one of the embodiments described herein in this aspect, the ionic polymer is characterized by a net charge (i.e., the difference between the number of anionic groups and the number of cationic groups) of from 1 to 6 electric charges per 1 kDa molecular weight of the polymer. In some embodiments, the ionic polymer has a net charge of from 1.5 to 4 charges per 1 kDa. In some embodiments, the ionic polymer has a net charge of from 2 to 3 charges per 1 kDa.

In some embodiments of any one of the embodiments described herein in this aspect, the ionic polymer is an anionic polymer, for example, a polymer characterized by a net negative charge of from 1 to 6 electric charges per 1 kDa molecular weight of the polymer.

In some embodiments of any one of the embodiments described herein in this aspect, the ionic polymer is a polysaccharide (which is an ionic polysaccharide).

As used herein throughout, the term “polysaccharide” refers to a polymer composed primarily (at least 50 weight percent) of monosaccharide units linked by glycosidic linkages.

As used herein, the term “monosaccharide” encompasses carbohydrates per se (having the formula Cn(H₂O)n, wherein n is at least 3, typically from 3 to 10), as well as derivatives thereof such as amino sugars, in which at least one hydroxyl group is replaced by an amine or amide group; sugar acids, in which one or two carbon atoms are oxidized to form a carboxylate group; acylated monosaccharides, in which at least one hydroxyl group and/or amine group is substituted by an acyl group (e.g., acetyl); and sulfated monosaccharides, in which at least one hydroxyl group is replaced by a sulfate group.

Examples of monosaccharides include, without limitation, hexoses (e.g., D-hexoses and/or L-hexoses) such as allose, altrose, glucose, mannose, gulose, idose, galactose, talose, psicose, fructose, sorbose and tagatose; pentoses (e.g., D-pentoses and/or L-pentoses) such as arabinose, lyxose, xylose, ribose, ribulose and xylulose; and hexose derivatives such as glucuronic acid, iduronic acid, manuronic acid, guluronic acid, glucosamine and N-alkyl derivatives thereof, galactosamine and N-alkyl derivatives thereof, N-acetylglucosamine, N-acetylgalactosamine, and monosulfated and disulfated N-acetylgalactosamine, glucuronic acid and iduronic acid.

As used herein, the phrase “glycosidic linkage” refers to a bond between a hemiacetal group of one compound (e.g., a monosaccharide monomer) and a hydroxyl group of another compound (e.g., another monosaccharide monomer).

Examples of ionic polysaccharides include, without limitation, hyaluronic acid, chondroitin sulfate, alginic acid, xanthan gum, chitosan and N-alkyl chitosan derivatives.

Hyaluronic acid is an anionic polysaccharide comprising anionic glucuronic acid monomer units along with non-ionic N-acetylglucosamine monomer units. Hyaluronic acid is an exemplary ionic (e.g., anionic) polymer.

Chondroitin sulfate is an anionic polysaccharide comprising anionic sulfated (e.g., monosulfated and/or disulfated) N-acetylgalactosamine, glucuronic acid and/or iduronic acid monomer units, and anionic glucuronic acid and/or iduronic acid monomer units, along with non-ionic N-acetylgalactosamine monomer units.

Alginic acid is an anionic polysaccharide comprising anionic mannuronic acid and guluronic acid monomer units.

Xanthan gum is an anionic polysaccharide comprising anionic glucuronic acid monomer units, along with non-ionic glucose and mannose monomer units (including acetyl and/or pyruvyl derivatives thereof).

Chitosan is a cationic polysaccharide comprising cationic glucosamine monomer units, optionally along with non-ionic N-acetylglucosamine monomer units. In N-alkyl chitosan derivatives, at least a portion of the glucosamine units comprise 1, 2 or 3 alkyl groups, preferably C₁₋₄ alkyl, attached to the nitrogen atom. In some embodiments of any one of the embodiments described herein, the alkyl groups attached to the nitrogen atoms are each independently methyl or ethyl. In some embodiments, the alkyls are methyl. In some embodiments, the N-alkylated monomer unit is N-trimethylglucosamine.

Herein, the terms “hyaluronic acid”, “chondroitin sulfate”, “alginic acid”, “xanthan gum”, “chitosan”, “N-alkyl chitosan derivatives” and any other ionic compounds named herein, encompass all salts of the named compounds along with the non-ionic forms (e.g., acid forms of the anionic polysaccharides, and the free base forms of the cationic polysaccharides).

In some embodiments of any one of the embodiments described in this aspect, the polysaccharide is in a form of a salt. In some embodiments, the salt is a pharmaceutically acceptable salt (e.g., an ophthalmically acceptable salt for an ophthalmic application as described herein, a salt suitable for parenteral administration for a parenteral application described herein).

In some embodiments of any one of the embodiments described in this aspect, the polysaccharide has from 0.2 to 1 charged groups per monosaccharide moiety. In some embodiments, the polysaccharide has from 0.2 to 0.9 charged groups per monosaccharide moiety. In some embodiments, the polysaccharide has from 0.3 to 0.7 charged groups per monosaccharide moiety. In some embodiments, the polysaccharide has from 0.4 to 0.6 charged groups per monosaccharide moiety. In some embodiments, the polysaccharide has about 0.5 charged groups per monosaccharide moiety.

It is to be appreciated that a monosaccharide moiety as described herein may comprise more than one charged group (e.g., a sulfate group and a carboxylate group).

In some embodiments of any one of the embodiments described in this aspect, the monosaccharide moieties comprise no more than one charged group, that is, 0 or 1 charged group.

In some embodiments of any one of the embodiments described in this aspect, the polysaccharide is characterized by a net charge (i.e., the difference between the number of anionic groups and the number of cationic groups) of from 0.2 to 1 electric charges per monosaccharide moiety. In some embodiments, the net charge is from 0.2 to 0.9 electric charges per monosaccharide moiety. In some embodiments, the net charge is from 0.3 to 0.7 electric charges per monosaccharide moiety. In some embodiments, the net charge is from 0.4 to 0.6 electric charges per monosaccharide moiety. In some embodiments, the net charge is about 0.5 electric charges per monosaccharide moiety.

In some embodiments of any one of the embodiments described herein, the water-soluble polymer comprises one or more biopolymers.

Herein, the term “biopolymer” refers to a polymer naturally occurring in a living organism. Examples of biopolymers include, without limitation, polynucleotides (e.g., RNA and DNA), polypeptides, polysaccharides and conjugates thereof (e.g., glycoproteins and proteoglycans comprising polypeptide and polysaccharide moieties). It is to be appreciated that biopolymers may optionally comprise many different species of related monomeric units (e.g., about 20 different types of amino acid residues and/or various types of monosaccharide moieties) with little or no repetition of the specific species of monomeric units, yet are considered polymers because at least some of the monomeric units are related in structure (e.g., being amino acid residues or monosaccharide moieties).

In some embodiments of any one of the embodiments described herein, the biopolymer(s) comprises a polypeptide (optionally attached to one or more saccharide moieties) and/or a polysaccharide.

Examples of suitable biopolymers comprising a polypeptide include, without limitation, mucins and lubricin.

Herein, the term “lubricin” refers to a proteoglycan (also known in the art as “proteoglycan 4”) of about 345 kDa. Human lubricin is encoded by the PRG4 gene. The lubricin optionally comprises a polypeptide sequence of isoform A and/or isoform B of lubricin, e.g., according to NCBI reference sequence NP_001121180.

Herein, the term “mucin” refers to a family of high molecular weight glycosylated proteins produced by many animals, and encompasses human mucins such as, for example, mucin 1 (e.g., according to NCBI reference sequence NP_001018016), mucin 2 (e.g., according to NCBI reference sequence NP_002448), mucin 3A (e.g., according to NCBI reference sequence NP_005951), mucin 3B, mucin 4 (e.g., according to NCBI reference sequence NP_004523), mucin 5AC, mucin 5B (e.g., according to NCBI reference sequence NP_002449), mucin 6 (e.g., according to NCBI reference sequence NP_005952), mucin 7 (e.g., according to NCBI reference sequence NP_001138478), mucin 8, mucin 12, mucin 13, mucin 15, mucin 16 (e.g., according to NCBI reference sequence NP_078966), mucin 17 (e.g., according to NCBI reference sequence NP_001035194), mucin 19, and mucin 20 (e.g., according to NCBI reference sequence NP_001269435).

The polysaccharide as described herein in this aspect may be a non-ionic polymer (as defined herein) or an ionic polymer (as defined herein), e.g., according to any of the embodiments described herein relating to an ionic polysaccharide.

Hyaluronic acid (e.g., according to any of the respective embodiments described herein) is a non-limiting example of a suitable polysaccharide as well as a non-limiting example of a suitable ionic (e.g., anionic) polymer.

In some embodiments of the present aspect, the ionic polymer is hyaluronic acid or a salt thereof.

Herein throughout, the term “at least one water-soluble polymer” means that the formulation or solution comprises one water-soluble polymer or a mixture of two or more water-soluble polymers. In some embodiments of any of the embodiments described herein, the formulation or solution as described herein in this aspect comprises one water-soluble polymer.

In some embodiments of any of the embodiments described in this aspect, the water-soluble polymers described herein comprise at least two water-soluble polymers according to any of the respective embodiments described herein. In some embodiments, the water-soluble polymers comprise at least three water-soluble polymers according to any of the respective embodiments described herein.

In some embodiments of any one of the embodiments described in this aspect, a molecular weight (i.e., average molecular weight or Mw, as known in the art) of the water-soluble polymer(s) is in a range of from 3 kDa to 10 MDa, including any intermediate values and subranges therebetween. In some embodiments, the molecular weight is from 10 kDa to 10 MDa, including any intermediate values and subranges therebetween. In some embodiments, the molecular weight is from 20 kDa to 5 MDa, including any intermediate values and subranges therebetween. In some embodiments, the molecular weight Mw is from 30 kDa to 2.5 MDa, including any intermediate values and subranges therebetween.

In some embodiments of any one of the embodiments described in this aspect, a molecular weight (i.e., average molecular weight or Mw) of the water-soluble polymer(s) is in a range of from 10 kDa to 1 MDa, including any intermediate values and subranges therebetween. In some embodiments, the molecular weight Mw is from 20 kDa to 500 kDa, including any intermediate values and subranges therebetween. In some embodiments, the molecular weight Mw is from 30 kDa to 250 kDa, including any intermediate values and subranges therebetween.

In some embodiments of any one of the embodiments described in this aspect, a molecular weight (i.e., average molecular weight or Mw) of the water-soluble polymer(s) is in a range of from 0.05 to 10 MDa, including any intermediate values and subranges therebetween. In some embodiments, the molecular weight Mw is from 0.05 to 5 MDa, including any intermediate values and subranges therebetween. In some embodiments, the molecular weight Mw is from 0.5 to 10 MDa, including any intermediate values and subranges therebetween. In some embodiments, the molecular weight Mw is from 0.5 to 5 MDa, including any intermediate values and subranges therebetween. In some embodiments, the water-soluble polymer(s) comprises an ionic polymer (according to any of the respective embodiments described herein), optionally an ionic polysaccharide, having an aforementioned molecular weight. In some embodiments, the ionic polymer is hyaluronic acid having an aforementioned molecular weight. In some embodiments of the present aspect, the water soluble polymer comprises a mixture of one or more water soluble polymers (e.g., anionic), each having a different Mw within the ranges as indicated herein.

In some embodiments, a concentration of a water-soluble polymer in the solution (according to any of the respective embodiments described in this aspect) is in a range of from 0.01 to 10 mg/ml, including any intermediate values and subranges therebetween. In some embodiments, the concentration is in a range of from 0.03 to 10 mg/ml, including any intermediate values and subranges therebetween. In some embodiments of this aspect, the concentration is in a range of from 0.1 to 10 mg/ml, including any intermediate values and subranges therebetween. In some embodiments, the concentration is in a range of from 0.3 to 10 mg/ml, including any intermediate values and subranges therebetween.

In some embodiments, a total concentration of water-soluble polymer(s) in the solution (according to any of the respective embodiments described in this aspect) is in a range of from 0.01 to 20 mg/ml. In some embodiments, the total concentration is in a range of from 0.03 to 20 mg/ml. In some embodiments, the total concentration is in a range of from 0.1 to 10 mg/ml. In some embodiments, the total concentration is in a range of from 0.3 to 10 mg/ml.

In some embodiments of this aspect of the present embodiments, a concentration of a water-soluble polymer in the solution (according to any of the respective embodiments described herein) in a range of from 0.01 to 1 mg/ml. In some embodiments, the concentration is in a range of from 0.03 to 1 mg/ml. In some embodiments, the concentration is in a range of from 0.1 to 1 mg/ml. In some embodiments, the concentration is in a range of from 0.3 to 1 mg/ml. In some embodiments, the water-soluble polymer is an ionic polymer and/or polysaccharide (e.g., as described herein in any one of the respective embodiments in this aspect), optionally hyaluronic acid.

In some embodiments of this aspect of the present embodiments, a concentration of each water-soluble polymer in the solution (according to any of the respective embodiments described herein) in a range of from 0.01 to 1 mg/ml. In some embodiments of this aspect, the concentration is in a range of from 0.03 to 1 mg/ml. In some embodiments of this aspect, the concentration is in a range of from 0.1 to 1 mg/ml. In some embodiments of this aspect, the concentration is in a range of from 0.3 to 1 mg/ml. In some embodiments, the water-soluble polymer is or hyaluronic acid.

In some embodiments of this aspect of the present embodiments, a total concentration of water-soluble polymer(s) in the solution (according to any of the respective embodiments described herein) in a range of from 0.01 to 2 mg/ml. In some embodiments of this aspect, the total concentration is in a range of from 0.03 to 2 mg/ml. In some embodiments of this aspect, the total concentration is in a range of from 0.1 to 1 mg/ml. In some embodiments of this aspect, the total concentration is in a range of from 0.3 to 1 mg/ml.

In some embodiments of any one of the embodiments described in this aspect, the water soluble polymer(s) comprises hyaluronic acid, at a concentration of less than 3 mg/ml. In some embodiments, the hyaluronic acid concentration is at least 0.01 mg/ml. In some embodiments of this aspect, the hyaluronic acid concentration is at least 0.03 mg/ml. In some embodiments of this aspect, the hyaluronic acid concentration is at least 0.1 mg/ml. In some embodiments of this aspect, the hyaluronic acid concentration is at least 0.3 mg/ml.

In some embodiments of any one of the embodiments described in this aspect, the water soluble polymer(s) comprises hyaluronic acid at a concentration of less than 0.75 mg/ml. In some embodiments of this aspect, the hyaluronic acid concentration is at least 0.01 mg/ml. In some embodiments of this aspect, the hyaluronic acid concentration is at least 0.03 mg/ml. In some embodiments, the hyaluronic acid concentration is at least 0.1 mg/ml. In some embodiments of this aspect, the hyaluronic acid concentration is at least 0.3 mg/ml.

In some embodiments of any one of the embodiments described in this aspect, the water soluble polymer(s) comprises hyaluronic acid at a concentration of less than 0.5 mg/ml. In some embodiments of this aspect, the hyaluronic acid concentration is at least 0.01 mg/ml. In some embodiments of this aspect, the hyaluronic acid concentration is at least 0.03 mg/ml. In some embodiments of this aspect, the hyaluronic acid concentration is at least 0.1 mg/ml. In some embodiments of this aspect, the hyaluronic acid concentration is at least 0.3 mg/ml.

In some embodiments of any one of the embodiments described in this aspect, the water soluble polymer(s) comprises hyaluronic acid at a concentration of less than 0.25 mg/ml. In some embodiments of this aspect, the hyaluronic acid concentration is at least 0.01 mg/ml. In some embodiments of this aspect, the hyaluronic acid concentration is at least 0.03 mg/ml. In some embodiments of this aspect, the hyaluronic acid concentration is at least 0.1 mg/ml.

In some embodiments of any one of the embodiments described in this aspect, the water soluble polymer(s) comprises hyaluronic acid at a concentration of less than 0.1 mg/ml. In some embodiments of this aspect, the hyaluronic acid concentration is at least 0.01 mg/ml. In some embodiments of this aspect, the hyaluronic acid concentration is at least 0.03 mg/ml.

In some embodiments of any one of the embodiments described in this aspect, a viscosity of the solution (which may reflect at least in part a concentration of water-soluble polymer(s) therein) is no more than 1000 cP (centipoise). In some embodiments of this aspect, the viscosity is no more than 500 cP. In some embodiments of this aspect, the viscosity is no more than 200 cP. In some embodiments of this aspect, the viscosity is no more than 100 cP. In some embodiments of this aspect, the viscosity is no more than 50 cP. In some embodiments of this aspect, the viscosity is no more than 20 cP. In some embodiments of this aspect, the viscosity is no more than 10 cP. In some embodiments of this aspect, the viscosity is no more than 5 cP. In some embodiments of this aspect, the viscosity is no more than 3 cP. In some embodiments of this aspect, the viscosity is no more than 2 cP. In some embodiments of this aspect, the solution is an aqueous solution having a viscosity described herein.

In the present aspect, viscosities of a solution are determined at a temperature of 20° C. and at a shear rate of 1 second-1 (unless indicated otherwise).

According to some of any of the embodiments described in this aspect, the composition or formulation or solution as described herein comprises one or more saccharides which are not polysaccharides as described herein, and which can be, for example, a monosaccharide as described herein, a disaccharide (composes of two monosaccharides linked therebetween as described herein) or an oligosaccharide, composed of from 3 to 10, or from 3 to 8, or from 3 to 6, monosaccharide units linked to one another as described herein.

An exemplary saccharide in the composition or formulation as described in this aspect is trehalose.

In some of any of the embodiments described in this aspect, an amount of the saccharide ranges from 0.1 to 10% by weight, or from 0.1 to 5, or from 1 to 10, or from 1 to 5, % by weight, of the total weight of the composition/formulation/solution, including any intermediate values and subranges therebetween.

In some embodiments of the present aspect, the ratio between the water-soluble polymers described herein and the saccharide described herein ranges from 100:1 to 1:100, or from 20:1 to 1:20, including any intermediate values and subranges therebetween.

Compositions (e.g., solutions) or formulations for use in accordance with the present invention thus may be formulated in conventional manner using one or more ophthalmically acceptable carriers, which facilitate processing of the water-soluble polymer(s) and/or liposomes into preparations which can be used as described herein. The water-soluble polymer(s) and/or liposomes described herein may be formulated as an aqueous solution per se. Additionally, the solution may be in the form of a suspension and/or emulsions (e.g., the aqueous phase of a suspension or water-in-oil, oil-in-water or water-in-oil-in-oil emulsion), for example, in order to increase the viscosity of the formulation.

According to some of any of the embodiments described herein, the compositions or formulations as described in this aspect of the present embodiments are for use in treatment of ocular discomfort.

According to some of any of the embodiments described herein, there is provided a method of treating an ocular discomfort, which is effected by ocular or ophthalmic administration of a composition or formulation as described in this aspect of the present embodiments.

In some embodiments of any one of the embodiments described herein relating to ocular discomfort, the ocular discomfort is associated with a contact lens. Association of a contact lens with ocular discomfort may be based on an observation of a contact lens wearer, for example, that discomfort occurs when contact lens are being worn, and/or based on a diagnosis by a physician (e.g., ophthalmologist), for example, that an ocular discomfort (e.g., chronic discomfort) is caused by a contact lens.

According to another aspect of embodiments of the invention, there is provided a method of treating ocular discomfort in a subject in need thereof, the method comprising ophthalmically administering to the subject an effective amount of the composition or formulation (e.g., solution) comprising liposomes and water-soluble polymer(s), as described herein in any one of the respective embodiments. According to another aspect of embodiments of the invention, there is provided a use of the composition or formulation (e.g., solution) comprising liposomes and water-soluble polymer(s), as described herein in any one of the respective embodiments, in the manufacture of a medicament for treating ocular discomfort.

### Drug Delivery:

According to some embodiments of the invention, there is provided a liposome comprising at least one bilayer-forming lipid, a polymeric compound according to any of the respective embodiments described herein, and a therapeutically active agent incorporated in the liposome and/or on the liposome, as described herein. The liposome, in some embodiments, is for use in delivering the therapeutically active agent to a subject in need thereof (e.g., to a bodily site of the subject).

According to an aspect of some embodiments of the invention, there is provided a use of a liposome comprising at least one bilayer-forming lipid, a polymeric compound according to any of the respective embodiments described herein, and a therapeutically active agent incorporated in the liposome and/or on the liposome, in the manufacture of a medicament for use in delivering the therapeutically active agent to a subject in need thereof (e.g., to a bodily site of the subject).

According to an aspect of some embodiments of the invention, there is provided a method of delivering a therapeutically active agent to a subject in need thereof (e.g., to a bodily site of the subject), the method comprising administering to the subject a liposome comprising at least one bilayer-forming lipid, a polymeric compound according to any of the respective embodiments described herein, and a therapeutically active agent incorporated in the liposome and/or on the liposome, thereby delivering the therapeutically active agent to a subject in need thereof.

In some embodiments of any of the embodiments according to any of the aspects described herein, the use of the liposome and/or method described herein is for treating a medical condition treatable by the therapeutically active agent (according to any of the respective embodiments described herein) in the subject.

In some embodiments of any of the embodiments according to any of the aspects described herein, delivering the therapeutically active agent comprises sustained release of the therapeutically active agent according to any of the respective embodiments described herein.

In some embodiments of any of the embodiments according to any of the aspects described herein, the liposome is selected as capable of sustained release of the therapeutically active agent according to any of the respective embodiments described herein.

As used herein, “delivery” or “delivering” of a therapeutically active agent or drug (which terms are used herein interchangeably) refers to administration of the therapeutically active agent to a subject while controlling duration and/or proportion of the agent at a desired bodily site, depending on a subject's condition (e.g., a bodily site at which the agent desirably exerts a therapeutic effect). Thus, the terms “delivery” and “delivering” (and grammatical variations thereof) encompass targeting of a therapeutically active agent to a specific bodily site, such that a higher proportion of the agent reaches said bodily site (e.g., using a suitable targeting moiety); and/or control over duration of a presence of such an agent in the body (e.g., in the blood)—for example, by sustained release—which may be associated with a duration of such an agent at a desired bodily site (even if in the absence of specific targeting to the bodily site).

As used herein, “sustained release” refers to a formulation of an agent which provides a gradual and/or delayed (“sustained”) release of the agent (e.g., from a reservoir such a liposome according to any of the respective embodiments described herein), which results in the agent being present in a bodily site (e.g., in the blood upon systemic administration, or in a bodily site to which the agent is locally administered) for a longer duration and/or at a later time (relative to administration) than if the agent is administered per se (via the same administration route).

For example, an agent administered per se (as opposed to a sustained release formulation) in the context of embodiments of the invention optionally refers to a formulation of the agent devoid of liposomes according to embodiments of the invention, and comprising the same carrier (if any) as the sustained release formulation.

In some embodiments, the sustained release is characterized by a concentration of therapeutically active agent (e.g., in the blood upon systemic administration, or in a bodily site to which the agent is locally administered) which is at least half of the maximal concentration (Cmax) for a time period which is at least 50% more than a corresponding time period (i.e., during which a concentration of an agent is at least half of the maximal concentration) an agent upon administration of the therapeutically effective agent per se (e.g., as defined herein) in an amount which results in the same maximal concentration. In some such embodiments, the time period (for sustained release) is at least 100% more than (i.e., twice) a corresponding time period (for the agent per se). In some embodiments, the time period (for sustained release) is at least 200% more than (i.e., 3-fold) a corresponding time period (for the agent per se). In some embodiments, the time period (for sustained release) is at least 400% more than (i.e., 5-fold) a corresponding time period (for the agent per se).

In some embodiments, the sustained release is characterized by a concentration of therapeutically active agent (e.g., in the blood upon systemic administration, or in a bodily site to which the agent is locally administered) which is at least half of the maximal concentration (Cmax) for a time period of at least 6 hours. In some such embodiments, the time period is at least 12 hours. In some embodiments, the time period is at least 24 hours. In some embodiments, the time period is at least 2 days. In some embodiments, the time period is at least 4 days. In some embodiments, the time period is at least one week. In some embodiments, the time period is at least 2 weeks. In some embodiments, the time period is at least 4 weeks.

Sustained release (according to any of the respective embodiments described herein), may allow, for example, for a regimen characterized by less frequent administration and/or by greater therapeutic efficacy of any given administration. The skilled person will be readily capable of determining a suitable frequency of administration for a given therapeutically active agent based on the duration of the sustained release (e.g., a time period during which the concentration of the agent is at least half of the maximal concentration, according to any of the respective embodiments described herein, and/or at least a minimal effective concentration), and the ratio between a desirable maximal concentration and a minimal effective concentration for the given agent (e.g., a “therapeutic window” of the agent).

In some such embodiments, the therapeutically active agent is an analgesic and/or an anti-inflammatory agent. In some such embodiments, the therapeutically active agent is usable in the treatment osteoarthritis, either alone or in combination with an additional therapeutically active agent.

In some of any of the embodiments described herein, the liposomes of the present embodiments are administered to a subject in need thereof in combination with an additional therapeutically active agent usable in the treatment of an indicated medical condition or a pharmaceutical composition comprising same. The additional therapeutically active agent can be incorporated in liposomes of the present embodiments, in other liposomes, which may have the same or different retention time of the agent, or can be simply mixed with an appropriate, liposome-free carrier.

According to some embodiments of the invention, the therapeutically effective agent is selected from the group consisting of an analgesic, an anti-inflammatory agent, an anti-proliferative agent, an anti-microbial agent, and a vaccine antigen.

In some such embodiments, the therapeutically active agent is an analgesic and/or an anti-inflammatory agent. In some such embodiments, the therapeutically active agent is usable in the treatment osteoarthritis, either alone or in combination with an additional therapeutically active agent.

According to some embodiments of the invention, the delivering is effected by parenteral systemic administration.

According to some embodiments of the invention, the delivering is effected by intra-articular administration.

According to some embodiments of the invention, the liposome is for use in the treatment of a synovial joint disorder.

According to some embodiments of the invention, the synovial joint disorder is selected from the group consisting of arthritis, bursitis, carpal tunnel syndrome, fibromyositis, gout, locked joint, tendinitis, traumatic joint injury, and joint injury associated with surgery.

According to some embodiments of the invention, the therapeutically active agent is an analgesic and/or anti-inflammatory agent.

According to some embodiments of the invention, the liposome is formulated as part of a pharmaceutical composition, which further comprises a pharmaceutically acceptable carrier.

According to some embodiments of the invention, the carrier comprises an aqueous liquid.

According to some embodiments of the invention, the pharmaceutical composition further comprises a water-soluble biopolymer.

According to some embodiments of the invention, the biopolymer comprises hyaluronic acid.

The skilled person will be readily capable of determining which medical condition(s) may be treatable by a given therapeutically active agent, as well as which therapeutically active agent(s) may be suitable for treating a given medical condition.

In some embodiments of any of the embodiments described herein, the liposome is for use in the treatment of a proliferative disease or disorder (e.g., cancer), and the therapeutically active agent is an anti-proliferative agent according to any of the respective embodiments described herein.

In some embodiments of any of the embodiments described herein, the liposome is for use in the treatment of an inflammatory disease or disorder (e.g., cancer), and the therapeutically active agent is an analgesic and/or anti-inflammatory agent according to any of the respective embodiments described herein.

In some embodiments of any of the embodiments described herein, the liposome is for use in the treatment of a synovial joint disorder (e.g., via systemic and/or intra-articular administration), optionally an inflammatory synovial joint disorder. Examples of synovial joint disorders treatable according to embodiments of the invention, include, without limitation, arthritis (e.g., osteoarthritis, rheumatoid arthritis and/or psoriatic arthritis), bursitis, carpal tunnel syndrome, fibromyositis, gout, locked joint (e.g., locked joint associated with osteochondritis dissecans and/or synovial osteochondromatosis), tendinitis, traumatic joint injury, and joint injury associated with surgery.

Joint injury associated with surgery may optionally be associated with surgery which directly inflicts damage on an articular surface (e.g., by incision), and/or surgery which damages an articular surface only indirectly. For example, surgery which repairs or otherwise affects tissue in the vicinity of the joint (e.g., ligaments and/or menisci) may be associated with joint injury due to altered mechanics in the joint.

Traumatic joint injury may optionally be injury caused directly by trauma (e.g., inflicted at the time of the trauma) and/or injury caused by previous trauma (e.g., a post-traumatic injury which develops sometime after the trauma).

### Process and Intermediates:

According to an aspect of some embodiments of the present invention there is provided a process of preparing a polymeric compound as described herein in any of the respective embodiments. As discussed herein, the process is selected so as to allow control of the composition of the polymeric portion of the polymeric compound.

According to some of any of the embodiments described herein, the process comprises contacting an initiator compound having Formula V:

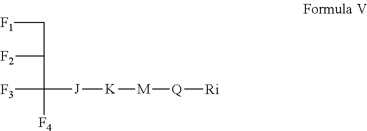

wherein:
F₁, F₂, F₃, F₄, J, K, M and Q are as defined for Formula IV; and
Ri is an electron transfer functional group,
with a plurality of monomers that form the —[Y-L-Z]n-[Y]m- polymeric backbone, wherein Y, L, Z, n and m are as described herein for Formula I,
under conditions that promote atom transfer radical polymerization (ATRP).

According to some of any of the embodiments described herein, Ri can be any functional group that is suitable for electron transfer radical polymerization, and is typically a group that is capable of forming a stable radical on its own. Exemplary such groups include halogens (halo), preferably chloro or bromo, more preferably bromo, although any other suitable groups are contemplated.

The term “stable radical” as used herein encompasses any chemical species that comprises an unpaired electron within its molecular or atomic structure but is relatively long-lived and less reactive compared to typical radicals. Typically, a stable radical is such that is capable of stabilizing the unpaired electron energetically.

Conditions that promote ATRP include any conditions known in the art, typically in the presence of radical forming reagents such as CuX′ or CuX′₂, wherein X′ is typically halogen, and a suitable ligand.

As discussed herein, the present inventors have uncovered that a better control over the polymerization process is achieved when the ATRP is ARGET-ATRP.

According to some of any of the embodiments described herein, the process is effected under conditions that promote ARGET-ATRP. Exemplary such conditions include any conditions known in the art, typically in the presence of CuX′₂, wherein X′ is typically halogen, a suitable ligand, and a reducing agent. Exemplary reducing agents and ligands that are suitable for use in the context of these embodiments are described in the Examples section that follows. Suitable solvents for carrying out ATRP or ARGET-ATRP processes include, without limitations, polar solvents such as alcohols (e.g., methanol and/or ethanol).

According to some of any of the embodiments described herein, the process is effected by ATRP, and M is other than amido.

According to some of any of the embodiments described herein, the process is effected by ATRP, and when M is amido, Q comprises an aryl group, as described herein.

According to some of any of the embodiments described herein, when M is amido, the process is effected by ARGET-ATRP.

According to some of any of the embodiments described herein, the process is effected at a temperature in a range of from 10° C. to 50° C., or from 15° C. to 50° C., or from 15° C. to 30° C., including any intermediate value and subranges therebetween. In some of any of the embodiments described herein, the process is effected at room temperature (i.e., ambient temperature; from about 20° C. to about 25° C.).

According to some of any of the embodiments described herein, contacting the initiator compound with the plurality of monomers is effected for a time period of from about 1 to about 48 hours, or from about 3 to about 48 hours, or from about 3 to about 36 hours, or from about 3 to about 24 hours, from about 4 to about 48 hours, or from about 4 to about 36 hours, or from about 4 to about 24 hours, from about 6 to about 48 hours, or from about 6 to about 36 hours, or from about 6 to about 24 hours including any intermediate value and subranges therebetween.

Exemplary procedures for performing ATRP process and ARGET-ATRP processes are described in the Examples section that follows. These procedures can be manipulated as desired by selecting an initiator compound, a mol ratio of the plurality of monomers to the initiator, by manipulating the catalyst solution, the ligand and/or the reducing, and by selecting a synthetic protocol as exemplified for Procedures 1, 2 and 3.

According to some of any of the embodiments described herein, the process is effected by contacting a catalyst solution that comprises a catalyst and a ligand with a solution comprising the initiator compound, preferably under inert atmosphere (e.g., argon), and with a solution of the plurality of monomers, at a mol ratio to the initiator compound selected to provide a desired length (number of repeating backbone units) of the obtained LPC.

According to some of any of the embodiments described herein, a mol ratio of the catalyst and the initiator is about 1:1.

According to some of any of the embodiments described herein, the process is effected by ATRP, using procedures known in the art. In an exemplary procedure (e.g., Procedure 1), an initiator compound as described herein is dissolved in a solvent (preferably a polar solvent such as, for example, dichloromethane, DCM), a solution comprising the catalyst and a ligand, in a polar solvent such as a protic solvent, e.g., alcohol such ethanol or methanol or even water, is added, followed by a solution of the plurality of monomers, also in a polar solvent as described. Optionally, the reaction is performed at a temperature of 20 to 50, or 20 to 40, or 30 to 50, or 30 to 40,° C.

According to some of any of the embodiments described herein, the process is effected by ARGET-ATRP, using procedures known in the art. In some of these embodiments, a catalyst solution is prepared by dissolving the catalyst and the ligand in a polar solvent (e.g., an alcoholic solvent such as MeOH or EtOH, preferably EtOH). In an exemplary procedure (e.g., Procedure 2), the catalyst solution is added to a mixture of the initiator compound, a reducing agent and a plurality of monomers, in a polar solvent as described herein (e.g., an alcoholic solvent). In another exemplary procedure (e.g., Procedure 3), the catalyst solution is added to a mixture of the initiator compound, and a plurality of monomers, in a polar solvent as described herein (e.g., an alcoholic solvent), and a reducing agent is thereafter added.

In some of any of the embodiments described herein, the mol ratio of the monomer (plurality of monomers) as described in any of the respective embodiments and in any combination thereof, and the initiator compound as described herein, is in a range of from 5:1 to 200:1, or from 10:1 to 150:1, or from 20:1 to 100:1, or from 30:1 to 75:1, including any intermediate values and subranges therebetween. This mol ratio determines the number of repeating units in the obtained polymeric compound (LPC).

In some embodiments, a mol ratio of 50:1 or lower (e.g., about 30:1 or about 25:1) provides short LPCs as described herein in of the respective embodiments, comprising less than 100, or less than 80, repeating units in the polymeric moiety (indicated by variable n in Formula I as described herein).

In some embodiments, a mol ratio of 60:1 or higher (e.g., from about 60:1 to about 80:1) provides longer LPCs as described herein in of the respective embodiments, comprising at least 80 repeating units (e.g., 80-120) in the polymeric moiety (indicated by variable n in Formula I as described herein).

According to some of any of the embodiments described herein, the process further comprises isolating the polymeric compound.

Isolating the obtained polymeric compound (e.g., a polymeric compound having Formula I as described herein in any of the respective embodiments and any combination thereof), can be effected by any work up process known in the art, preferably in the context of ATRP processes, including, for example, TFF, column chromatography and/or precipitation. Exemplary such procedures are described in the Examples section that follows.

According to some of any of the embodiments described herein, isolating the polymeric compound does not involve acidification, namely, exposing the polymeric compound to an acidic environment.

According to some of any of the embodiments described herein, isolating the polymeric compound is performed by precipitation, namely, precipitating the polymeric compound from the polymerization reaction mixture by means of, for example, contacting the mixture with an anti-solvent in which the polymeric compound is not soluble, at a ratio in a range of from 2:1 to 50:1, anti-solvent: reaction mixture, including any intermediate values and subranges therebetween. Exemplary anti-solvents include, but are not limited to ketones such as acetone, dimethoxyethane (DME), chloroform (CHCl₃), dichloromethane (DCM), tetrachloroethylene (C₂Cl₄), dimethylcarbonate (DMC), diethylcarbonate (DEC), and methyl tert-butyl ether (MTBE). In exemplary embodiments, the anti-solvent is a ketone, for example, acetone.

According to some of any of the embodiments described herein, isolating the polymeric compound further comprises, prior to and/or subsequent to the precipitation, column chromatography.

According to some of any of the embodiments described herein, there are provided compounds represented by Formula V as described herein in any of the respective embodiments and any combination thereof.

According to some of any of the embodiments described herein, there are provided compounds represented by Formula V as described herein in any of the respective embodiments and any combination thereof, for use as intermediates in preparing a polymeric compound as described herein in any of the respective embodiments and any combination thereof.

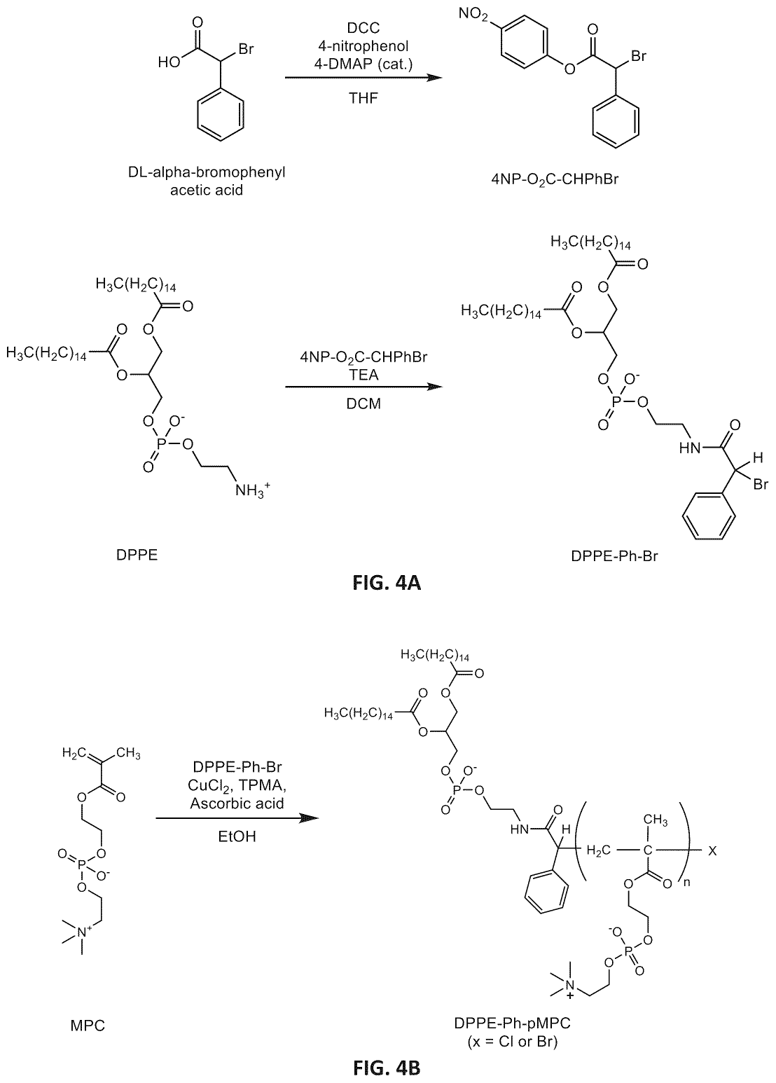

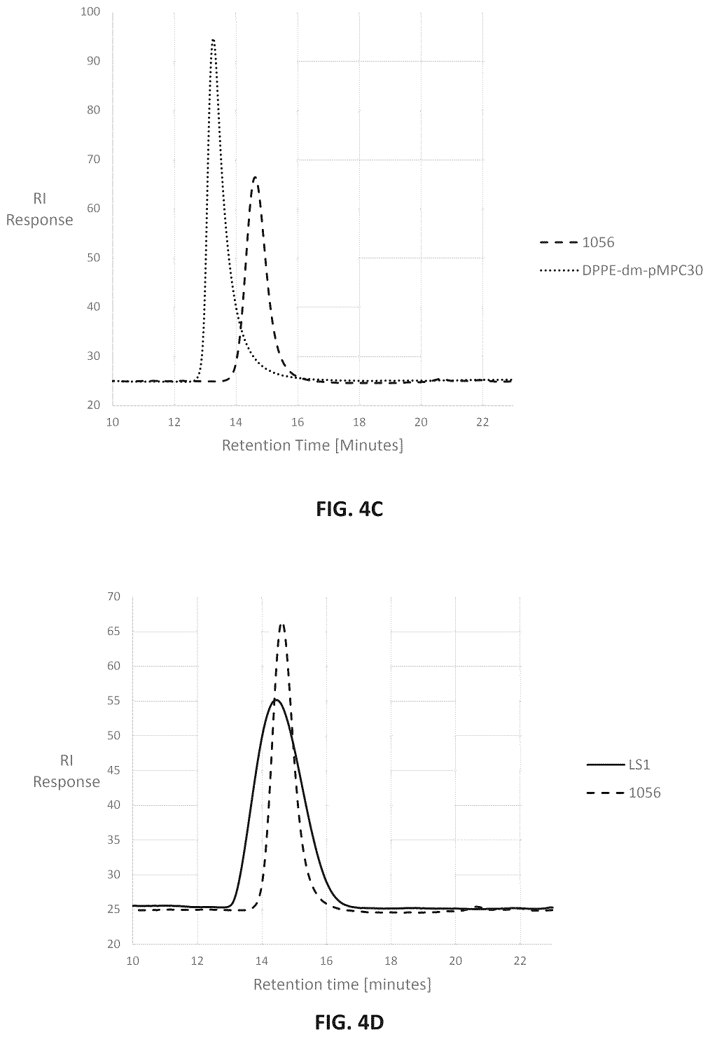

Exemplary compound of Formula V and their preparation are described in the Examples section that follows, and some are shown in FIGS. 4A, 5A and 6A.

### Additional Definitions

Herein, the term “hydrocarbon” describes an organic moiety that includes, as its basic skeleton, a chain of carbon atoms, substituted mainly by hydrogen atoms. The hydrocarbon can be saturated or non-saturated, be comprised of aliphatic, alicyclic or aromatic moieties, and can optionally be substituted by one or more substituents (other than hydrogen). A substituted hydrocarbon may have one or more substituents, whereby each substituent group can independently be, for example, cycloalkyl, alkenyl, alkynyl, aryl, heteroaryl, heteroalicyclic, amine, halide, sulfonate, sulfoxide, phosphonate, hydroxy, alkoxy, aryloxy, thiohydroxy, thioalkoxy, thioaryloxy, oxo, cyano, nitro, azo, azide, sulfonamide, carboxy, thiocarbamate, urea, thiourea, carbamate, amide, and hydrazine. The hydrocarbon can be an end group or a linking group, as these terms are defined herein. The hydrocarbon moiety is optionally interrupted by one or more heteroatoms, including, without limitation, one or more oxygen, nitrogen and/or sulfur atoms. In some embodiments of any of the embodiments described herein relating to a hydrocarbon, the hydrocarbon is not interrupted by any heteroatoms.

Preferably, the hydrocarbon moiety has 1 to 20 carbon atoms. Whenever a numerical range; e.g., “1 to 20”, is stated herein, it implies that the group, in this case the alkyl group, may contain 1 carbon atom, 2 carbon atoms, 3 carbon atoms, etc., up to and including 20 carbon atoms.

Herein, the term “alkyl” describes a saturated aliphatic hydrocarbon end group, as defined herein, including straight chain and branched chain groups. Preferably, the alkyl group has 1 to 20 carbon atoms. More preferably, the alkyl is a medium size alkyl having 1 to 10 carbon atoms. Most preferably, unless otherwise indicated, the alkyl is a lower alkyl having 1 to 4 carbon atoms. The alkyl group may be substituted or non-substituted. Substituted alkyl may have one or more substituents, whereby each substituent group can independently be, for example, cycloalkyl, alkenyl, alkynyl, aryl, heteroaryl, heteroalicyclic, amine, halide, sulfonate, sulfoxide, phosphonate, hydroxy, alkoxy, aryloxy, thiohydroxy, thioalkoxy, thioaryloxy, cyano, nitro, azo, azide, sulfonamide, carboxy, thiocarbamate, urea, thiourea, carbamate, amide, and hydrazine.

The term “alkylene” describes a saturated aliphatic hydrocarbon linking group, as this term is defined herein, which differs from an alkyl group, as defined herein, only in that alkylene is a linking group rather than an end group.

Herein, the term “alkenyl” describes an unsaturated aliphatic hydrocarbon end group which comprises at least one carbon-carbon double bond, including straight chain and branched chain groups. Preferably, the alkenyl group has 2 to 20 carbon atoms. More preferably, the alkenyl is a medium size alkenyl having 2 to 10 carbon atoms. Most preferably, unless otherwise indicated, the alkenyl is a lower alkenyl having 2 to 4 carbon atoms. The alkenyl group may be substituted or non-substituted. Substituted alkenyl may have one or more substituents, whereby each substituent group can independently be, for example, cycloalkyl, alkynyl, aryl, heteroaryl, heteroalicyclic, amine, halide, sulfonate, sulfoxide, phosphonate, hydroxy, alkoxy, aryloxy, thiohydroxy, thioalkoxy, thioaryloxy, cyano, nitro, azo, azide, sulfonamide, carboxy, thiocarbamate, urea, thiourea, carbamate, amide, and hydrazine.

Herein, the term “alkynyl” describes an unsaturated aliphatic hydrocarbon end group which comprises at least one carbon-carbon triple bond, including straight chain and branched chain groups. Preferably, the alkynyl group has 2 to 20 carbon atoms. More preferably, the alkynyl is a medium size alkynyl having 2 to 10 carbon atoms. Most preferably, unless otherwise indicated, the alkynyl is a lower alkynyl having 2 to 4 carbon atoms. The alkynyl group may be substituted or non-substituted. Substituted alkynyl may have one or more substituents, whereby each substituent group can independently be, for example, cycloalkyl, alkenyl, aryl, heteroaryl, heteroalicyclic, amine, halide, sulfonate, sulfoxide, phosphonate, hydroxy, alkoxy, aryloxy, thiohydroxy, thioalkoxy, thioaryloxy, cyano, nitro, azo, azide, sulfonamide, carboxy, thiocarbamate, urea, thiourea, carbamate, amide, and hydrazine.

The term “cycloalkyl” describes an all-carbon monocyclic or fused ring (i.e., rings which share an adjacent pair of carbon atoms) group where one or more of the rings does not have a completely conjugated pi-electron system. The cycloalkyl group may be substituted or non-substituted. Substituted cycloalkyl may have one or more substituents, whereby each substituent group can independently be, for example, alkyl, alkenyl, alkynyl, cycloalkyl, aryl, heteroaryl, heteroalicyclic, amine, halide, sulfonate, sulfoxide, phosphonate, hydroxy, alkoxy, aryloxy, thiohydroxy, thioalkoxy, thioaryloxy, cyano, nitro, azo, azide, sulfonamide, carboxy, thiocarbamate, urea, thiourea, carbamate, amide, and hydrazine. The cycloalkyl group can be an end group, as this phrase is defined herein, wherein it is attached to a single adjacent atom, or a linking group, as this phrase is defined herein, connecting two or more moieties.

The term “aryl” describes an all-carbon monocyclic or fused-ring polycyclic (i.e., rings which share adjacent pairs of carbon atoms) end group (as this term is defined herein) having a completely conjugated pi-electron system. The aryl group may be substituted or non-substituted. Substituted aryl may have one or more substituents, whereby each substituent group can independently be, for example, alkyl, alkenyl, alkynyl, cycloalkyl, aryl, heteroaryl, heteroalicyclic, amine, halide, sulfonate, sulfoxide, phosphonate, hydroxy, alkoxy, aryloxy, thiohydroxy, thioalkoxy, thioaryloxy, cyano, nitro, azo, azide, sulfonamide, carboxy, thiocarbamate, urea, thiourea, carbamate, amide, and hydrazine. Phenyl and naphthyl are representative aryl end groups.

The term “heteroaryl” describes a monocyclic or fused ring (i.e., rings which share an adjacent pair of atoms) group having in the ring(s) one or more atoms, such as, for example, nitrogen, oxygen and sulfur and, in addition, having a completely conjugated pi-electron system. Examples, without limitation, of heteroaryl groups include pyrrole, furan, thiophene, imidazole, oxazole, thiazole, pyrazole, pyridine, pyrimidine, quinoline, isoquinoline and purine. The heteroaryl group may be substituted or non-substituted. Substituted heteroaryl may have one or more substituents, whereby each substituent group can independently be, for example, alkyl, alkenyl, alkynyl, cycloalkyl, aryl, heteroaryl, heteroalicyclic, amine, halide, sulfonate, sulfoxide, phosphonate, hydroxy, alkoxy, aryloxy, thiohydroxy, thioalkoxy, thioaryloxy, cyano, nitro, azo, azide, sulfonamide, carboxy, thiocarbamate, urea, thiourea, carbamate, amide, and hydrazine. The heteroaryl group can be an end group, as this phrase is defined herein, where it is attached to a single adjacent atom, or a linking group, as this phrase is defined herein, connecting two or more moieties. Representative examples are pyridine, pyrrole, oxazole, indole, purine and the like.

The term “arylene” describes a monocyclic or fused-ring polycyclic linking group, as this term is defined herein, and encompasses linking groups which differ from an aryl or heteroaryl group, as these groups are defined herein, only in that arylene is a linking group rather than an end group.

The term “heteroalicyclic” describes a monocyclic or fused ring group having in the ring(s) one or more atoms such as nitrogen, oxygen and sulfur. The rings may also have one or more double bonds. However, the rings do not have a completely conjugated pi-electron system. The heteroalicyclic may be substituted or non-substituted. Substituted heteroalicyclic may have one or more substituents, whereby each substituent group can independently be, for example, alkyl, alkenyl, alkynyl, cycloalkyl, aryl, heteroaryl, heteroalicyclic, amine, halide, sulfonate, sulfoxide, phosphonate, hydroxy, alkoxy, aryloxy, thiohydroxy, thioalkoxy, thioaryloxy, cyano, nitro, azo, azide, sulfonamide, carboxy, thiocarbamate, urea, thiourea, carbamate, amide, and hydrazine. The heteroalicyclic group can be an end group, as this phrase is defined herein, where it is attached to a single adjacent atom, or a linking group, as this phrase is defined herein, connecting two or more moieties. Representative examples are piperidine, piperazine, tetrahydrofuran, tetrahydropyran, morpholine and the like.

As used herein, the terms “amine” and “amino” describe both a —NRxRy end group and a —NRx- linking group, wherein Rx and Ry are each independently hydrogen, alkyl, cycloalkyl, aryl, heteroaryl or heteroalicyclic, as these terms are defined herein. When Rx or Ry is heteroaryl or heteroalicyclic, the amine nitrogen atom is bound to a carbon atom of the heteroaryl or heteroalicyclic ring. A carbon atom attached to the nitrogen atom of an amine is not substituted by —O or ═S, and in some embodiments, is not substituted by any heteroatom.

The amine group can therefore be a primary amine, where both Rx and Ry are hydrogen, a secondary amine, where Rx is hydrogen and Ry is alkyl, cycloalkyl, aryl, heteroaryl or heteroalicyclic, or a tertiary amine, where each of Rx and Ry is independently alkyl, cycloalkyl, aryl, heteroaryl or heteroalicyclic.

The terms “hydroxy” and “hydroxyl” describe a —OH group.

The term “alkoxy” describes both an —O-alkyl and an —O-cycloalkyl end group, or —O-alkylene or —O-cycloalkyl linking group, as defined herein.

The term “aryloxy” describes both an —O-aryl and an —O-heteroaryl end group, or an —O-arylene- linking group, as defined herein.

The term “thiohydroxy” describes a —SH group.

The term “thioalkoxy” describes both an —S-alkyl and an —S-cycloalkyl end group, or —S-alkylene or —S-cycloalkyl linking group, as defined herein.

The term “thioaryloxy” describes both an —S-aryl and an —S-heteroaryl end group, or an —S-arylene- linking group, as defined herein.

The terms “cyano” and “nitrile” describe a —C≡N group.

The term “nitro” describes an —NO₂ group.

The term “oxo” describes a ═O group.

The term “azide” describes an —N═N⁺═N⁻ group.

The term “azo” describes an —N═N—Rx end group or —N═N═ linking group, with Rx as defined herein.

The terms “halide” and “halo” refer to fluorine, chlorine, bromine or iodine.

The term “phosphate” refers to a —O—P(═O)(ORx)-OR_{Y} end group, or to a —O—P(═O)(ORx)-O— linking group, where Rx and R_{Y} are as defined herein.

The terms “phosphonyl” and “phosphonate” refer to an —P(═O)(ORx)-OR_{Y} end group, or to a —P(═O)(ORx)-O— linking group, where Rx and R_{Y} are as defined herein. The term “phosphinyl” refers to a —PRxR_{Y} group, where Rx and R_{Y} are as defined hereinabove.

The term “sulfoxide” or “sulfinyl” describes a —S(═O)—Rx end group or —S(═O)— linking group, where Rx is as defined herein.

The terms “sulfonate” and “sulfonyl” describe a —S(═O)₂-Rx end group or —S(═O)₂— linking group, where Rx is as defined herein.

The terms “sulfonamide” and “sulfonamido”, as used herein, encompass both S-sulfonamide and N-sulfonamide end groups, and a —S(═O)₂—NRx- linking group.

The term “S-sulfonamide” describes a —S(═O)₂-NRxR_{Y} end group, with Rx and R_{Y} as defined herein.

The term “N-sulfonamide” describes an RxS(═O)₂—NR_{Y}— end group, where Rx and R_{Y} are as defined herein.

The term “carbonyl” as used herein, describes a —C(═O)—Rx end group or —C(═O)— linking group, with Rx as defined herein.

The term “acyl” as used herein, describes a —C(═O)—Rx end group, with Rx as defined herein.

The term “thiocarbonyl” as used herein, describes a —C(═S)—Rx end group or —C(═S)— linking group, with Rx as defined herein.

The terms “carboxy” and “carboxyl”, as used herein, encompasses both C-carboxy and O-carboxy end groups, and a —C(═O)—O—linking group.

The term “C-carboxy” describes a —C(═O)—ORx end group, where Rx is as defined herein.

The term “O-carboxy” describes a —OC(═O)—Rx end group, where Rx is as defined herein.

The term “urea” describes a —NRxC(═O)—NRyRw end group or —NRxC(═O)—NRy- linking group, where Rx and Ry are as defined herein and Rw is as defined herein for Rx and Ry.

The term “thiourea” describes a —NRx-C(═S)—NRyRw end group or a —NRx-C(═S)—NRy- linking group, with Rx, Ry and Ry as defined herein.

The terms “amide” and “amido”, as used herein, encompasses both C-amide and N-amide end groups, and a —C(═O)—NRx- linking group.

The term “C-amide” describes a —C(═O)—NRxRy end group, where Rx and Ry are as defined herein.

The term “N-amide” describes a RxC(═O)—NRy- end group, where Rx and Ry are as defined herein.

The term “carbamyl” or “carbamate”, as used herein, encompasses N-carbamate and O-carbamate end groups, and a —OC(═O)—NRx- linking group.

The term “N-carbamate” describes a RyOC(═O)—NRx- end group, with Rx and Ry as defined herein.

The term “O-carbamate” describes an —OC(═O)—NRxRy end group, with Rx and Ry as defined herein.

The term “thiocarbamyl” or “thiocarbamate”, as used herein, encompasses O-thiocarbamate, S-thiocarbamate and N-thiocarbamate end groups, and a —OC(═S)—NRx- or —SC(═O)—NRx- linking group.

The terms “O-thiocarbamate” and “O-thiocarbamyl” describe a —OC(═S)—NRxRy end group, with Rx and Ry as defined herein.

The terms “S-thiocarbamate” and “S-thiocarbamyl” describe a —SC(═O)—NRxRy end group, with Rx and Ry as defined herein.

The terms “N-thiocarbamate” and “N-thiocarbamyl” describe a RyOC(═S)NRx- or RySC(═O)NRx- end group, with Rx and Ry as defined herein.

The term “guanidine” describes a —RxNC(═N)—NRyRw end group or —RxNC(═N)—NRy- linking group, where Rx, Ry and Rw are as defined herein.

The term “hydrazine”, as used herein, describes a —NRx-NRyRw end group or —NRx-NRy- linking group, with Rx, Ry, and Rw as defined herein.

As used herein the term “about” refers to ±10%, and optionally ±5%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention.

Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non-limiting fashion.

### Materials and Methods

### Materials

Dichloromethane (DCM), chloroform, 2-Bromoisobutyryl bromide (BIBB), triethylamine (TEA), N,N,N′,N″,N″-pentamethyldiethylenetriamine (PMDETA), Azobisisobutyronitrile (AIBN) 0.2M solution in toluene, trimethylolpropane (TMP), ascorbic acid, propargyl alcohol, 1-hexadecanethiol, CuBr, anhydrous copper chloride (II)(CuCl₂) and phosphate buffer saline (PBS) were obtained from Sigma-Aldrich.

Distearoylphosphatidylethanolamine (DSPE), Dipalmitoylphosphatidylethanolamine (DPPE) and dipalmitoylphosphatidylglycerol (DPPG) exemplary bilayer-forming lipids, were obtained from Lipoid.

Methanol (MeOH), ethanol (EtOH), ethyl acetate (EtOAc), and n-hexane were obtained from Bio-Lab.

Hydrogenated soy phosphatidylcholine (HSPC) was obtained from Lipoid GmbH.

O-(2-methacryloyloxyethyl)phosphorylcholine (MPC) was obtained from known vendors (e.g., Biocompatible Corporation (UK) or Sigma Aldrich®).

Dicyclohexylcarbodiimide (DCC), pyridine, palmitoyl chloride and 4-nitrophenol were obtained from Thermo Scientific.

Tetrahydrofuran (THF) was obtained from known vendors (e.g., Fisher Chemicals or Bio-Lab).

4-dimethylaiminopyridien (4-DMAP) was obtained from Merck.

Tris(2-pyridylmethyl)(TPMA) was obtained from Angene.

All other solvents and reagents were obtained from known vendors.

HPLC measurements were performed using Agilent 1100 system equipped with an evaporative light scattering (ELS) detector using a reversed phase C8 column (Kinetex C8, 150×4.6 mm, 5 μm, 100 A) equilibrated to 30° C.

GPC measurements were performed using an Agilent 1100 system equipped with a refractive index detector using a combination of PFG columns (PFG Column Guard+100 Å PFG single pore+300 Å PFG single pore) equilibrated to 40° C.

Particles size was evaluated by dynamic light scattering (DLS) measurements using a Zetasizer Nano instrument by Malvern Panalytical. Liposome samples were diluted×10 in PBS to a final volume of 1 mL, transferred to a plastic cuvette and equilibrated to 25° C., followed by 3 replicate measurements. The mean Z-average diameter and mean polydispersity index (PDI) were reported along with their standard deviations.

Z-Potential was determined using a Zetasizer Nano instrument by Malvern Panalytical. Samples were diluted ×100-200 in water to a final volume of 800 μL, transferred to a disposable folded capillary cuvette and equilibrated to 25° C., followed by 3 replicate measurements. The Z-potential and its standard deviation are reported.

DSC was performed using a Nano DSC instrument by TA. Generally, a 300 μL sample is degassed for 10 minutes and transferred to the capillary sample cell of the instrument. 300 μL reference solution is degassed for 10 minutes and transferred to the capillary reference cell. A DSC thermogram is recorded at a temperature range of 50°-60° in 3 replicates. The thermograms are analyzed using the NanoAnalyze and the main phase transition temperature (gel to liquid) is reported with its standard deviation.

CryoTEM was performed using a Tencai G2 cryo TEM microscope by FEI. 3 μL liposome samples were applied on plasma-treated microscope grids followed by freezing of the samples in ethane. Image analysis was performed by a combination of visual inspection and the ImageJ software.

Stability study: To test stability in an accelerated condition, it is customary to expose to 40° C.\75% relative humidity (RH). At these conditions, the liposome membrane is in the liquid phase. In that state, the probability of changes in liposome size due to higher interactions and possible fusion is higher.

A real-time stability study was performed under a controlled environment (at 15-25° C. and below 65% RH) for a period of one week, a month, and two months. An accelerated stability study for formulations comprising 0.7% polymer was carried out in a temperature chamber at 40±2° C. and 75±5% RH in an outsourcing organization. The sampling time points were 14 and 28 days. Samples were stored at room temperature unless otherwise noted.

Additional experimental methods are described hereinbelow.

### Example 1

### General Design and Purification Modification

While pursuing to produce lipid-containing polymer compound (LPC)-containing liposomes of Reference Example 1 in a large-scale process, different approaches for the preparation of the LPCs were tested.

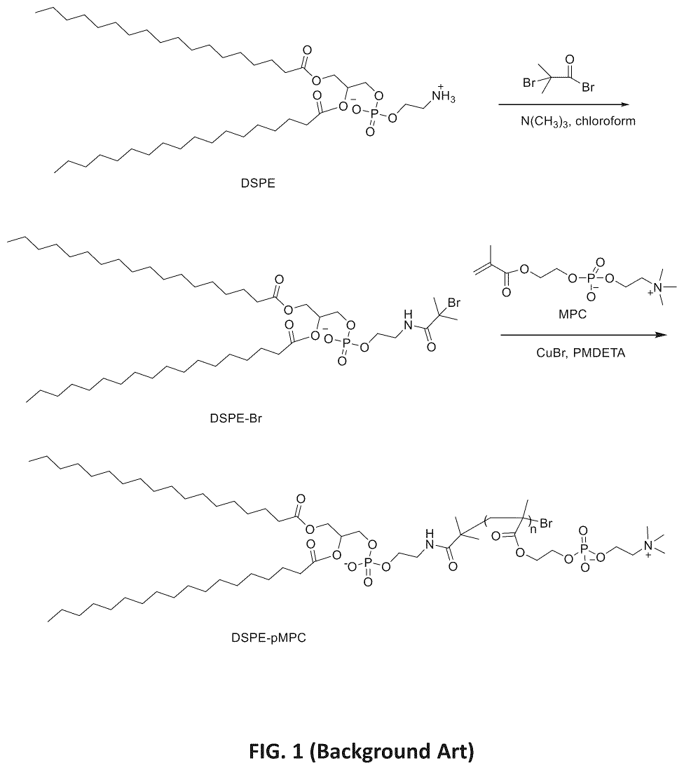

WO 2017/109784, by some of the present inventors, describes the preparation and use of lipid polymer conjugates, LPCs, composed of a polymeric chain having phosphocholine-containing pendant groups, conjugated to a lipid moiety such as a phospholipid (e.g., glycerophospholipid). WO 2017/109784 describes in detail a preparation of a phospholipid with a polymerized phosphocholine derivative, from the phospholipid DSPE (distearoylphosphatidylethanolamine) and the phosphocholine derivatized-MPC (O-(2-methacryloyloxyethyl)phosphorylcholine), as shown in Background Art FIG. 1. The obtained polymeric product is referred to as DSPE-pMPC (distearoylphosphatidylethanolamine-substituted poly(O-(2-methacryloyloxyethyl)phosphorylcholine)). DSPE-pMPC may be regarded as a polymeric phosphatidylcholine analog in view of the similarity between the structure of each MPC unit and the structure of a phosphocholine head group in phosphatidylcholine.

The LPCs described in WO 2017/109784 were shown to form, when combined with bilayer-forming lipids, stabilized liposomes, and to perform well in various applications that utilize such liposomes.

Due to the advantages associated with the LPCs described in WO 2017/109784 and the liposomes formed thereby, and the myriad of uses thereof, the present inventors have searched for further approaches for preparing such LPCs, which may enable efficient and controlled production of these LPCs.

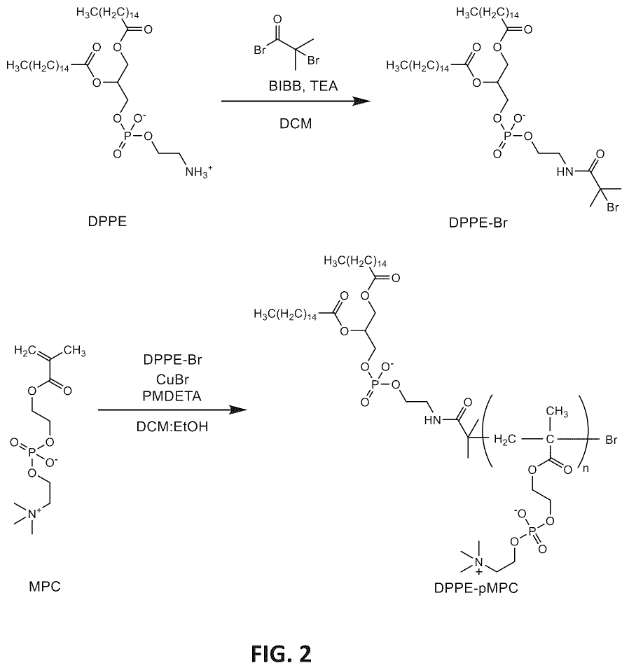

The present inventors have started from the process described in WO 2017/109784 using 1,2-dipalmitoyl-sn-glycero-3-phosphorylethanolamine (DPPE)-based initiator as an exemplary ATRP initiator, which is also referred to herein as DPPE-dm-Br (dm=dimethyl), and which initiates the polymerization of MPC to provide the LPC DPPE-dm-pMPC. The initiator and the LPC were prepared following the procedure described in WO 2017/109784, as depicted in FIG. 2, and as follows.

### Synthesis of 2,3-bis(palmitoyloxy)propyl (2-(2-bromo-2-methylpropanamido)ethyl) phosphate (DPPE-dm-Br ATRP Initiator)

Triethylamine (TEA; 3.3 mL, 24.0 mmol) was added to 210 mL of dry dichloromethane (DCM) containing 6.92 grams (10.0 mmol) of dipalmitoylphosphatidylethanolamine (DPPE), and the mixture was stirred at room temperature for 0.5 hour. 2-bromoisobutyryl bromide (BIBB; 2.76 grams, 1.5 mL, 12.0 mmol) was then injected into the solution, and the obtained mixture was stirred at 40° C. for 3 hours. The mixture was allowed to reach ambient temperature and thereafter washed thrice with HCl 1M, dried over anhydrous Na₂SO₄, filtered, and evaporated to about 50 mL. Then, the concentrated DCM solution was diluted into 500 mL MeOH and the mixed organic solution was placed in the fridge (4° C.) for at least 2 hours to allow precipitation of the product. The obtained white solid was filtered-off, washed twice with cold MeOH, and dried under vacuum to constant weight.

### Synthesis of DPPE-Dm-pMPC Via Atom-Transfer Radical Polymerization (ATRP)

Atom-transfer radical polymerization (ATRP)(also referred to herein as “Procedure 1”) was performed as described in WO 2017/109784, and as depicted in FIG. 2. In brief, DPPE-dm-Br (1.18 gram, 1.4 mmol) was dissolved in 8 mL of DCM in a Schlenk flask equipped with a magnetic stirring bar. The solution was purged with Argon to deoxygenate the flask and then, under positive Argon pressure, PMDETA (0.616 mL, 3.0 mmol) and CuBr (0.212 gram, 1.5 mmol) were added to the flask. Separately, 3.32 gram (11.2 mmol) of MPC were dissolved in 8 mL of EtOH and this solution was added to the flask as well. The flask was purged for additional 30 minutes, and the reaction was stirred overnight at 40° C. Then, reaction was allowed to reach room temperature and it was quenched by opening the flask to air. Reaction mixture was added to cold diethyl ether, which caused the LPC product to precipitate. The obtained blue solid was filtered-off, washed freely with diethyl ether and dried under vacuum to constant weight.

The obtained reaction mixture includes a blue-colored precipitation which indicates that the LPC precipitates along with the copper-ligand complex, thus requiring further purification steps to provide the desired LPC.

While in WO 2017/109784 it is taught that purification of the LPC is performed by dialysis, the present inventors have sought for alternative purification methods. The following methodologies were conceived:

### (i) Tangential Flow Filtration (TFF)

Acidification of LPCs: This step is assumed to aid dissolving the copper complex by protonation and to facilitate copper complex removal by TFF, yet is optional.

Separation by Tangential flow filtration (TFF): The dried crude polymer was dissolved in EtOH (25 mL per 1 gram of crude LPC) and then the alcoholic solution was diluted with water to give a ratio of 2:1 in favor of water. 1 molar (M) Aqueous HCl solution was added to the LPC solution to a final concentration of 90 millimolar (mM) and the solution was loaded into a closed circulating loop with a low molecular weight membrane that is impermeable to the polymer, with the aim of removing small molecule impurities (e.g., Cu, PMDETA and unreacted MPC).

### (ii) Silica Gel Column Chromatography:

Another option of purifying the LPC is by a silica column using MeOH as eluent. The silica is first washed with MeOH to remove soluble impurities. Once the silica is equilibrated and ready to use, the LPC crude is dissolved in MeOH and loaded onto the column. The eluted LPC is detected using TLC (eluent=DCM:MeOH 1:1 v/v, Rf=0.7) with I₂ staining. Fractions that contain the LPC are combined, evaporated to constant weight under high vacuum. The dried LPC is dissolved in water (15 mL per 1 gram of LPC) and lyophilized to afford the LPC as white crystalline solid.

(iii) Precipitation:

Separation by precipitation is performed by dropwise addition of the crude LPC ethanolic solution to acetone using a ratio of 1:10 v/v ethanol/acetone. The formed precipitate is filtered-off, washed with acetone, re-dissolved in ethanol, evaporated, dried, re-dissolved in water and then lyophilized.

Samples of DPPE-dm-pMPC obtained using the above-described purification protocols were characterized by HPLC and GPC, as follows:

HPLC of LPC samples: each LPC sample was dissolved in methanol (MeOH) to give a 1 milligram per milliliter (mg/mL) solution and was then analyzed using a mobile phase gradient change from 52.6% of H₂O+20 millimolar (mM) ammonium acetate (NH₄Ac):MeOH 95:5 v/v and 47.4% McOH+20 mM NH₄Ac to 100% MeOH+20 mM NH₄Ac in 7 minutes at 1 mL/minute.

GPC of LPC samples: each LPC sample was dissolved in HFIP+50 mM KTFA (GPC eluent) to give a concentration of 5 mg/mL. A flow marker (benzoic acid, 1 mg/mL) was also added to the solution. LPC samples were analyzed using a flow of 1 mL/minute at 40° C.

The obtained data revealed that purification protocols that involve acidification of the crude LPC (e.g., along with the TFF protocol and the column chromatography protocol, described above) adversely affect the structure uniformity and polydispersity of the polymeric conjugate (data not shown).

### Example 2

### Reagent Modifications

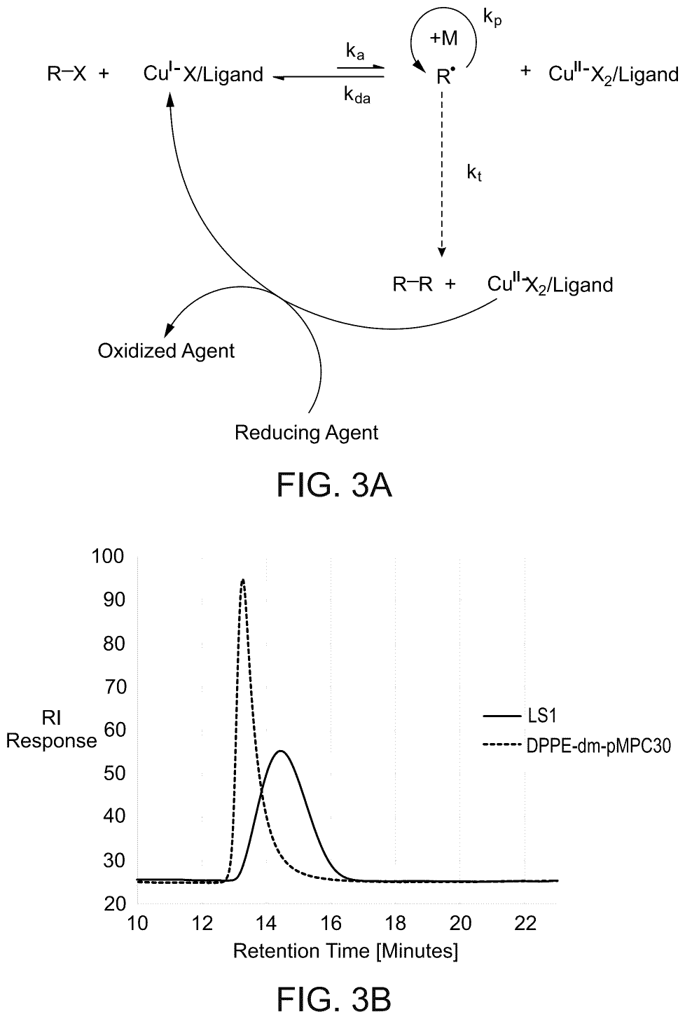

In order to further facilitate the product separation and optimize the preparation of the LPC, the present inventors have conceived manipulating the ATRP procedure by using activators regenerated by electron transfer (ARGET) ATRP. ARGET-ATRP is a variation of regular ATRP with scale-up possibilities due to a lower amount of the Cu catalyst required to initiate and maintain controlled polymerization. The Cu catalyst is added to the reaction in its oxidized state (2+) as CuX₂ (X being typically a halo such as Cl or Br) and is being constantly regenerated back to its active polymerizing state (1+) using a reducing agent, as depicted schematically in FIG. 3A. This process is less sensitive than normal ATRP towards the presence of oxygen and therefore simplifies the reaction setup.

While lower quantity of the copper catalyst is used in the ARGET-ATRP compared to the ATRP procedure, stoichiometric amounts of the reducing agent are required to provide the target degree of polymerization (DPn; the mol ratio between initiator to monomer). In addition, the reducing agent (both in its reduced and oxidized form) needs to be inert to all other reaction components.

Examples of safe, cheap and commercially available reducing agents that can be employed in ARGET-ATRP include, without limitation, ascorbic acid (vitamin C), sodium ascorbate (NaAsc), Calcium Ascorbate (Ca₂Asc), and hydrazine (mono hydrate or dissolved in ethanol), tin-2-ethyhexanoate (Tin-2EH) and glucose. For polymerization of MPC, both CuBr₂ and CuCl₂ (preferably anhydrous) work well together with TPMA as an exemplary ligand, while the constant reduction of the catalyst is performed using ascorbic acid [see, for example, Adler et al., Biomaterials Science, 2021, 9, 5854-5867]. Other ligands are also contemplated, including, for example, N,N,N′,N′-tetrakis(2-pyridinylmethyl)-1,2-ethanediamine (TPEN; CAS No. 16858-02-9).

In all of the synthetic protocols described herein for preparing a lipid-polymer conjugate, the type of the lipid moiety can be manipulated by preparing a respective initiator (which comprises the selected lipid moiety). Further, the length of the polymeric moiety (number of repeating units of e.g., MPC) is controlled by the mol equivalents of the selected monomer (e.g., MPC) with respect to the initiator (a mol ratio of the monomer relative to the initiator).

The following exemplary general procedure was used, employing the following catalyst solution: CuX₂ (X═Cl or Br, 0.10 mol equivalent to the initiator) was dissolved in an alcoholic solvent (e.g., MeOH or EtOH, preferably EtOH). TPMA (0.20 mol equivalent to the initiator) was added to the CuCl₂ green solution, and the obtained metal-ligand complex solution was mixed for at least 15 minutes.

In one exemplary procedure (also referred to herein as Procedure 2), into an oven-dried round-bottomed Schlenk flask equipped with a magnetic stirring bar, an initiator (1 mol equivalent) and Ascorbic Acid (1.41 mol equivalent to initiator) were added, followed by addition of a solution of MPC (25-100 mol equivalents to initiator; e.g., 30 mol equivalents to initiator) in the selected solvent (e.g., EtOH). The total solvent (e.g., EtOH) volume used in the reaction was set according to the amount of MPC to give a concentration of 15% w/v. The Catalyst solution was then added to the Schlenk flask, the flask was sealed with rubber septum and all contents were mixed together to form a greenish clear solution.

In an alternative procedure (also referred to herein as Procedure 3), into an oven-dried round-bottomed Schlenk flask equipped with a magnetic stirring bar, an initiator (1 mol equivalent) was added, followed by addition of a solution of MPC (25-100 mol equivalents to initiator; e.g., 30 mol equivalents to initiator) in the selected solvent (e.g., EtOH), as above, and an addition of the catalyst solution. The flask was sealed with rubber septum and all contents were mixed together to form a greenish clear solution, and, after all of the solids completely dissolved, Sodium Ascorbate (1.00 mol equivalent to the initiator) was added.

Once all components were introduced to the flask, it was purged from oxygen by bubbling Argon gas for 30 minutes through the solution. The Schlenk valve was thereafter tightly closed, and the polymerization mixture was allowed to stir at ambient temperature for 2-24 (e.g., 5-6) hours.

In both Procedures 2 and 3, polymerization was quenched by opening the flask to air. The obtained solution was optionally filtered-off using a Buchner funnel equipped with a Whatman paper. The obtained solution (or filtrate) was added dropwise into acetone while stirring, to precipitate the polymer product as a white solid. The volume of acetone was set to be 10 times greater than the volume of the alcoholic solvent (e.g., EtOH). The formed slurry was allowed to vigorously stir for additional 30 minutes, and the precipitate was thereafter filtered-off through a glass funnel, and the precipitation vessel and the polymer were washed thrice with acetone. The obtained polymer (LPC), which had a paste-like texture, was purified by dissolving it in EtOH and/or deionized water (15 mL per 1 gram of LPC), while optionally removing impurities by filtration. When EtOH was used, the solvents were evaporated and the residue was dissolved in purified water, filtered from insoluble impurities, and further purified by TFF (8-10 diafiltrations with deionized water). When water was used, the obtained aqueous solution was filtered from insoluble impurities and then further purified using TFF (8-10 diafiltrations with DIW). The aqueous LPC solutions were placed in a −80° C. freezer for at least 2 hours and then lyophilized for at least 48 hours (e.g., 96 hours).

HPLC and GPC analyses were performed on LPC sample batches produced from DPPE-dm-Br, using:
(1) the original procedure (Procedure 1)—ATRP polymerization and separation using precipitation and silica column (LS1 and LS2, based on Example 1); and
(2) ARGET-ATRP-prepared according to the exemplary ARGET-ATRP procedures described hereinabove (Procedures 2 and 3).

FIG. 3B presents overlaid GPC chromatograms of ATRP using a DPPE-dm-Br initiator (LS1; Procedure 1) and ARGET-ATRP using a DPPE-dm-Br initiator (Procedure 2).

The data obtained for the DPPE-dm-pMPC LPC obtained by ARGET-ATRP shows a symmetric peak with narrow distribution (dashed plot), while batch LS1 (solid plot) resulted in a wider peak distribution. This comparison between the polymerization methods suggests that the synthesis via ARGET-ATRP yields a product of lower polydispersity. Analyzing batch DPPE-dm-pMPC30 by GPC also indicated that the LPC comprises 180-200 MPC units.

Optional modifications to the above-described procedures include selecting CuCl₂ as a catalyst (over CuBr₂) and/or performing the reaction at room temperature, so as to allow activation of the initiator during the initial steps of the polymerization and slow down the polymerization propagation.

### Example 3

### Initiator Modification

An additional approach for the efficient preparation of LPCs involves manipulating the chemical nature of the initiator.

Without being bound to any particular theory, it has been assumed that the presence of an amide group adjacent to a di-methylated and halogenated α-carbon accounts for a relatively low initiation rate of the polymerization. Indeed, the most common initiators according to related literature feature an ester group at the respective position [see, for example, Limer & Haddleton, Macromolecules, 2006, 39:1353-1358; and Adams & Young, Journal of Polymer Science: Part A: Polymer Chemistry, 2008, 6082-6090].

The present inventors have designed and prepared an analogous initiator, comprising a phenyl substituent on the α-carbon, with the aim of increasing the activity of the initiator (see, for example, Tang et al., JACS, 2008, 130:10702-10713). The newly designed phenyl-containing initiator is referred to herein as DPPE-Ph-Br (Ph=phenyl).

### Preparation of DPPE-Ph-Br Initiator:

In one exemplary procedure, DPPE-Ph-Br was prepared via a two-step reaction as follows:
(a) Steglich esterification of DL-α-bromophenylacetic with 4-nitrophenol to afford the active 4-nitrophenol ester (4NP—O₂C—CHPhBr); and (b) reacting the resulting ester with DPPE to obtain DPPE-Ph-Br, as depicted in FIG. 4A. Reacting the resulting ester with other glycerophospholipids can provide additional Ph-Br-based initiators.

### (a) Synthesis of 4-nitrophenyl 2-bromo-2-phenylacetate (4NP—O₂C—CHPhBr)

DL-α-bromophenylacetic acid (4.30 grams, 20 mmol, 1.0 mol equivalent), 4-nitrophenol (3.06 grams, 22 mmol, 1.1 mol equivalent) and 4-DMAP (0.244 gram, 2 mmol, 0.1 mol equivalent) were weighed into a round bottomed flask equipped with a magnetic stirring bar. THF (20 mL per 1 gram of DL-α-bromophenylacetic acid) was added to the flask and stirring was turned-on to completely dissolve all solids. The flask was thereafter sealed, cooled to about 5° C. using an ice-bath, and dicyclohexylcarbodiimide (DCC; 4.54 grams, 22 mmol, 1.1 mol equivalent) was added. After 10 minutes of stirring, the ice-bath was removed, and the flask was allowed to reach room temperature. The reaction mixture was allowed to stir for 3 more hours at ambient temperature and then its content was gravitationally filtered-off into an evaporation flask using a common filtration paper. The reaction flask and the filtered urea byproduct were washed twice with DCM (25 mL) and the combined filtrates were evaporated to dryness using a rotary evaporator. The obtained yellowish oily crude mixture was re-dissolved in DCM (10-15 mL) and loaded on a silica column with DCM as eluent. Collected fractions that contained the product (identified using TLC with UV light at 254 nm, eluent=DCM, Rf=0.95) were combined, evaporated to dryness, and placed under high vacuum overnight. The product was obtained as a clear and slightly yellowish oil, and upon overnight drying under high vacuum it crystalized to give 5.05 grams of a pale yellow solid in 75% yield.

### (b) Synthesis of 2,3-bis(palmitoyloxy)propyl (2-(2-bromo-2-phenylacetamido)ethyl) phosphate (DPPE-Ph-Br ATRP Initiator)

4NP—O₂C—CHPhBr (5.05 grams, 15.0 mmol, 1.2 mol equivalent) was dissolved using DCM (15 mL per 1 gram 4NP—O₂C—CHPhBr) in a round bottomed flask equipped with a magnetic stirring bar. Then, DPPE (8.66 grams, 12.5 mmol, 1.0 mol equivalent) was added to the flask and washed down with additional DCM (10 mL per 1 gram DPPE). TEA (4.2 mL, 30.0 mmol, 2.4 mol equivalents) was added and the flask was heated to 50° C. using a water bath to afford gentle solvent reflux. At the beginning of the reaction, a foam was obtained in the solution, which was cleared following one hour of reflux and vigorous stirring. The reaction was refluxed for additional 2 hours and was then allowed to cool to room temperature. The content of the flask was transferred into a separation funnel and the organic phase was washed thrice with equivalent volumes of 1 Molar (M) HCl for each wash. The combined aqueous acidic phase was extracted twice with DCM (50 mL). The combined organic DCM phase was dried over anhydrous Na₂SO₄, filtered, and evaporated to 30-50 mL. The DPPE-Ph-Br product was precipitated by adding the concentrated DCM solution to a laboratory bottle filled with stirring MeOH (75 mL per 1 gram of DPPE) and placing this bottle in the fridge for at least 2 hours. The precipitated product was filtered-off, washed twice with cold MeOH (50 mL) and allowed to dry with suction still turned-on for at least 10 minutes. Finally, the precipitate was dried overnight under high vacuum to give 9.82 grams of DPPE-Ph-Br in 88% yield.

In another exemplary procedure, DPPE-Ph-Br was prepared as follows:

### (a) Activation of αBPA with NHS Towards Coupling Reaction with DPPE

DL-α-bromophenylacetic acid (αBPA, 7.53 grams, 35.0 mmol, 1.4 mol equivalent to DPPE) and N-hydroxysuccinimide (NHS, 4.32 grams, 37.5 mmol, 1.5 mol equivalent to DPPE) were added into a 100 mL round-bottomed flask equipped with a magnetic stirrer and a powder funnel. Tetrahydrofuran (THF, 40 mL) was added through the powder funnel and stirring was turned on to completely dissolve both solids. Diisoproylcarbodiimide (DIC, 5.1 mL, 32.5 mmol, 1.3 mol equivalent to DPPE) was added thereafter to the flask using a syringe, the flask was sealed, and the reaction was allowed to stir for 1 hour at room temperature. Then, the precipitated DIC-urea byproduct was filtered-off gravitationally through a standard filtration paper and the reaction flask and filtration paper were washed with 5 mL THF. The obtained clear solution was kept aside for the next step.

### (b) Reacting DPPE with the NHS Ester of αBPA

In parallel, a 500 mL round-bottomed flask was equipped with a magnetic stirrer and a powder funnel. 1,2-dipalmitoyl-sn-glycero-3-phosphoethanolamine (DPPE, 17.3 grams, 25 mmol) was added to the flask, following by addition of chloroform (CHCl₃, 175 mL) through the powder funnel. Then, triethylamine (TEA, 3.8 mL, 27.5 mmol, 1.1 mol equivalent to DPPE) was added to the flask using a syringe, and the contents of the flask were strongly stirred while heating the flask to 70° C. After complete dissolution was achieved, the flask was allowed to gradually cool down to 35° C., and then the clear NHS ester solution from Step (a) was added in a single portion, and the reaction was allowed to proceed for 1 hour at 35° C. Thereafter, the flask was allowed to cool down to room temperature, e CHCl₃ was evaporated under reduced pressure and the obtained oily residue was transferred into a 1 L separation funnel using a total of 500 mL Ethyl Acetate (EtOAc). The organic phase in the separation funnel was washed thrice with HCl 1M (250 mL), thrice with Brine (250 mL), dried over anhydrous MgSO₄, and filtered gravitationally using a standard filtration paper into a 1 L lab bottle to separate any solid particulates. The lab bottle with the clear EtOAc solution was placed in a −20° C. freezer for 2 hours, during which the DPPE-Ph-Br product precipitated as a white solid. DPPE-Ph-Br was filtered-off using a Buchner filtration system and the collected product was washed twice with cold EtOAc (25 mL). The product was dried to constant weight under high vacuum. A yield of 80-90% of pure DPPE-Ph-Br was obtained.

As the above procedure includes in situ activation of αBPA without the need to perform any purification to the active ester, the DPPE-Ph-Br can alternatively be prepared in a one-pot reaction. Activation is performed by turning αBPA into an active NHS ester using diisoproylcarbodiimide (DIC) as a coupling reagent.

### Synthesis of DPPE-Ph-pMPC Via ARGET-ATRP

MPC was polymerized from the exemplary DPPE-Ph-Br initiator via ARGET-ATRP using anhydrous CuCl₂ as the catalyst, tris(2-pyridylmethyl)(TPMA) as the ligand, and ascorbic acid or Sodium Ascorbate (NaAsc) as the reducing agent, to obtain DPPE-Ph-pMPC, as described hereinabove and as exemplified schematically in FIG. 4B.

Catalyst solution was prepared as follows: CuCl₂ (7.1 mg, 0.053 mmol, 0.053 mol equivalent to the initiator) was weighed into a 20 mL scintillation vial and dissolved in 2 mL EtOH. TPMA (31 mg, 0.105 mmol, 0.105 mol equivalent to the initiator) was added to the CuCl₂ green solution. The metal-ligand complex solution was allowed to mix for at least 15 minutes.

In one exemplary procedure (also referred to herein as “Procedure 2”), DPPE-Ph-pMPC was prepared as follows.

Separately, To an oven-dried round-bottomed Schlenk flask equipped with a magnetic stirring bar, DPPE-Ph-Br (888 mg, 1.0 mmol, 1 mol equivalent) and ascorbic acid (93 mg, 0.527 mmol, 0.527 mol equivalent to the initiator) were added, followed by adding a solution of MPC (e.g., 8.86 grams, 30 mmol, 30 mol equivalents to the initiator, or otherwise an amount equivalent to the desired length of the polymer) in a total of 56 mL EtOH. The Catalyst solution was then added to the Schlenk flask, the flask was sealed with rubber septum and all contents were mixed together to form a greenish clear solution. The flask was then purged from oxygen by bubbling Argon gas for 30 minutes through the solution using a stainless-steel needle. Polymerization was allowed by stirring the reaction mixture at room temperature for 24 hours. Polymerization was quenched by opening the flask to air. The obtained turbid solution was filtered-off using a Buchner funnel equipped with a Grade 3 Whatman paper filter. The Schlenk flask and the precipitate were washed with EtOH (2×10 mL) and passed through the filtered solid. The combined filtrate was added dropwise to Acetone to thereby precipitate the polymer product as a white solid. The volume of Acetone was set to be 10 times greater than the volume of EtOH. The formed slurry was allowed to stir vigorously for additional 30 minutes, and was thereafter filtered-off using a glass funnel. The precipitation vessel and the polymer were washed with additional Acetone (2×100 mL). The obtained polymer (LPC), which had a paste-like texture, was transferred to a glass beaker and re-dissolved in EtOH. This solution was filtered through a regular filtration paper to an evaporation flask and all organic solvents were evaporated to dryness. The dried LPC was placed under high vacuum overnight, and was thereafter re-dissolved in purified water (15 mL per 1 gram of the obtained LPC), while heating for 30 minutes at 50° C. The solution was thereafter allowed to cool down to room temperature and was filtered through 0.22 μm PES membrane. Divided aqueous LPC solutions were placed in the −80° C. freezer for at least 2 hours and then lyophilized for at least 48 hours.

In an alternative exemplary procedure (also referred to herein as “Procedure 3”), DPPE-Ph-pMPC was prepared as follows:

Catalyst solution was prepared as follows: anhydrous CuCl₂ (152 mg, 1.129 mmol, 0.10 mol equivalent to DPPE-Ph-Br) was weighed into a 20 mL scintillation vial and dissolved in 15 mL EtOH. TPMA (656 mg, 2.258 mmol, 0.20 mol equivalent to DPPE-Ph-Br) was added to the CuCl₂ green solution, and the vial was manually shaken until all TPMA dissolved causing the solution's color to change from light to dark green.

Separately, into an oven-dried 1 L round-bottomed Schlenk flask equipped with a magnetic stirring bar and a powder funnel, DPPE-Ph-Br (10.025 grams, 11.289 mmol, 1.0 mol equivalents to DPPE-Ph-Br) was added, followed by a solution of MPC (100.0 grams, 338.7 mmol, 30 mol equivalents to DPPE-Ph-Br or otherwise, as described herein) in 568 mL EtOH. The Catalyst solution was then also added to the Schlenk flask, and the 20 mL vial was washed with an additional 15 mL portion of EtOH. At this point, the flask was temporarily sealed with a rubber septum and all contents were strongly mixed together to form a greenish clear solution. NaAsc (2.24 grams, 11.289 mmol, 1.0 mol equivalent to DPPE-Ph-Br) was added to the flask, the flask was sealed with a rubber septum, and then it was purged from oxygen by bubbling Argon gas for 30 minutes through the solution using a stainless-steel needle. Polymerization was thereafter performed by allowing the reaction mixture to stir at room temperature for 5-6 hours.

Polymerization was quenched by opening the flask to air. The reaction was filtered-off from NaAsc leftovers using a Buchner funnel equipped with a Grade 3 Whatman paper filter. The Schlenk flask was washed twice with EtOH (10 mL) and each wash was passed through the filter paper. The combined filtrate was added dropwise into stirring Acetone to thereby precipitate the polymer product as a white solid. The volume of Acetone was set to be 10 times greater than the volume of EtOH (6.7 L). The formed suspension was stirred strongly for an additional 15 minutes and thereafter the polymer LPC product was allowed to settle down to the bottom of the precipitation vessel for 1 hour. Then, the precipitate was filtered-off using a sinter glass funnel. The precipitation vessel and the collected polymer were washed twice with additional acetone (250 mL) and each time all solvents were allowed to completely drain with suction. The LPC product was allowed to dry in the air for 5-10 minutes and then it was transferred into a 3 L glass beaker and re-dissolved directly in 1500 mL DIW with stirring for 30 minutes. The obtained aqueous solution was filtered through a 0.2 μm PES filter and was further purified using a TFF system. After performing 8-10 diafiltrations with DIW, the purified aqueous solution was divided into weighed vessels suitable for lyophilization, which were placed in the −80° C. freezer for at least 2 hours. Then, the LPC was lyophilized for 96 hours to afford a white crystalline solid. Batch 1056 was prepared by polymerizing MPC with the exemplary DPPE-Ph-Br initiator by ARGET-ATRP and purifying only by precipitation, as described hereinabove in Procedure 2.

Batch 1056 was compared by HPLC (not shown) and GPC measurements with the LPC prepared under the same conditions, using DPPE-dm-Br initiator, as described hereinabove in Procedure 1 (referred to herein as DPPE-dm-pMPC30). The GPC analysis is presented in Table 1 below and in FIG. 4C.

TABLE 1

| | | Obtained DPn |
|---|---|---|
| Initiator | LPC product | (#MPC units) |
| DPPE-dm-Br | DPPE-dm-pMPC30 | 180-200 |
| DPPE-Ph-Br | Batch 1056 | 50-80 |

While the polymerization via ARGET-ATRP using each of the DPPE-dm-Br and DPPE-Ph-Br initiators (see, FIG. 4C, dashed and dotted black plots, respectively), both provide quality products, by examining the efficiency of each initiator according to GPC, it can be seen that DPPE-dm-Br provides lower initiation rate compared to DPPE-Ph-Br under the same conditions: the same mol ratio of 1:30 (initiator: MPC) led to 180-200 MPC units in DPPE-dm-pMPC, and 50-80 MPC units in DPPE-Ph-pMPC.

These data demonstrate the efficiency of the novel DPPE-Ph-Br initiator and its ability to control polymerization.

FIG. 4D further emphasizes the new synthetic approach by comparing DLS data obtained for DPPE-dm-pMPC prepared by ATRP (LS1; Procedure 1) and DPPE-Ph-pMPC prepared by ARGET-ATRP (batch 1056; Procedure 2).

LPC batches synthesized using DPPE-Ph-Br as an initiator according to Procedure 3 were analyzed by GPC. The GPC analyses data are presented in Table 2 below, demonstrating that finely defined LPC products were obtained reproducibly.

TABLE 2

| Batch | LPC Mn | LPC Mw | LPC | DPn |
|---|---|---|---|---|
| No. | [kDa] | [kDa] | PDI | (#MPC units) |
| IM23-1248 | 18.94 | 18.96 | 1.001 | 61 |
| IM23-1252 | 18.17 | 18.22 | 1.002 | 59 |
| IM23-1254 | 18.62 | 18.67 | 1.003 | 60 |
| IM23-1255 | 18.77 | 18.85 | 1.004 | 61 |

### Example 4

### Newly Designed Modified LPC Initiators

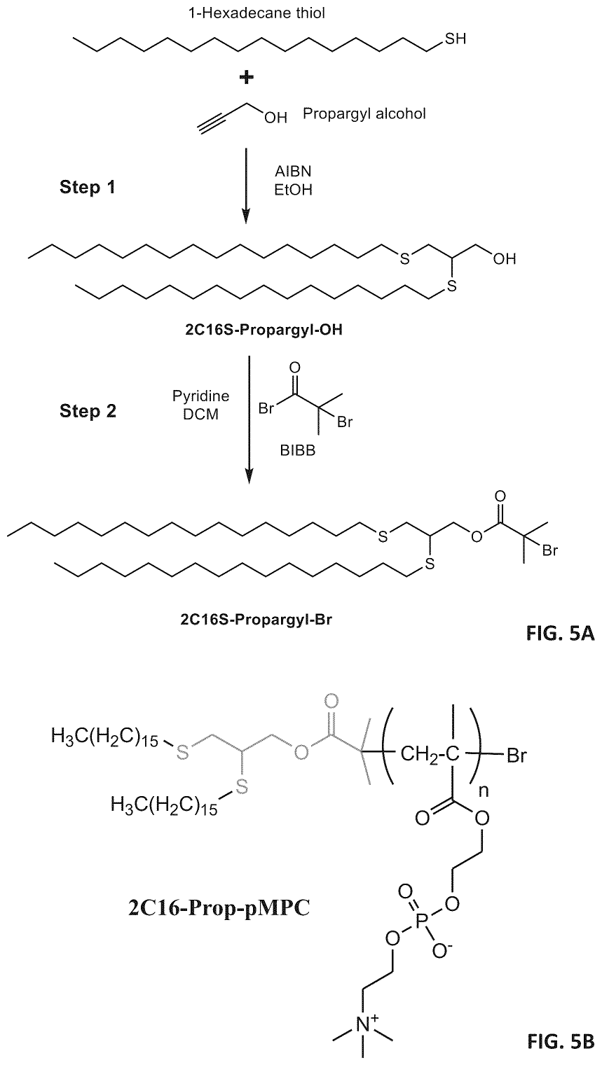

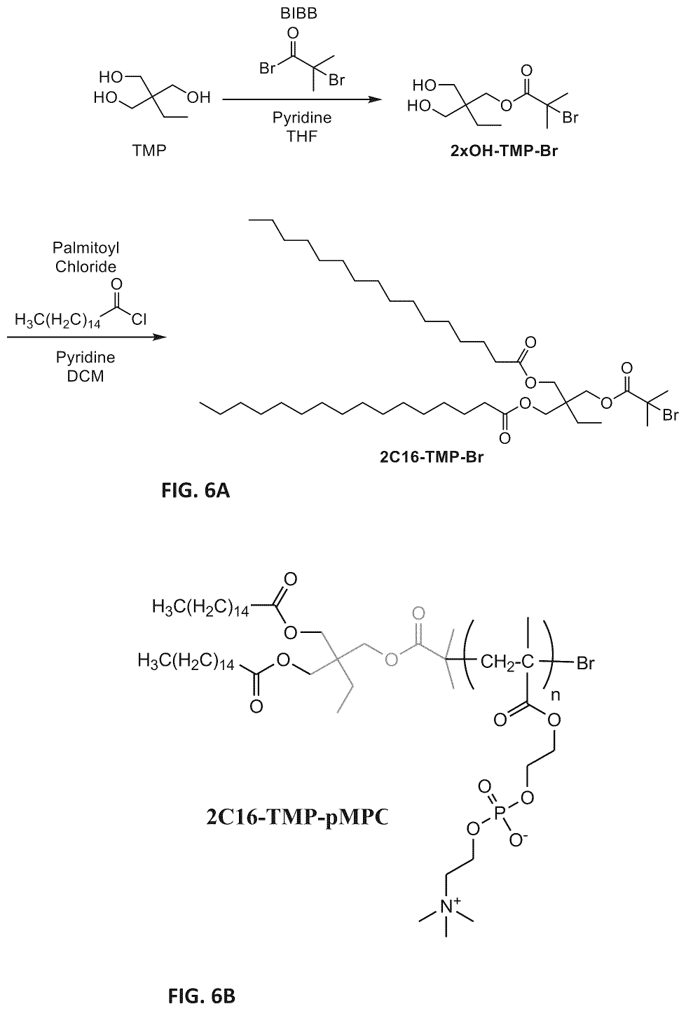

In a search for further improved initiators for preparing LPCs, the present inventors have designed such conjugates that feature an ester bond rather than an amide bond, per the above discussion, and have prepared and practiced newly designed initiators that feature a bis-thiol moiety (denoted as 2C16S-Propargyl-Br or 2C16S-Prop-Br) and a trimethylolpropane (denoted 2C16-TMP-pMPC), as exemplary moieties that link fatty chains to the ester bond. These exemplary LPCs are presented schematically in FIGS. 5B and 6B, respectively.

### Preparation of 2C16S-Propargyl-Br

The exemplary 2C16S-Propargyl-Br initiator was prepared via a two-step procedure from (a) propargyl alcohol and 1-hexadecanethiol through a thiol-yne reaction; and (b) reacting the formed bis-thiolated alcohol with α-bromoisobutyryl bromide (BIBB), as described herein and as depicted schematically in FIG. 5A.

### Synthesis of 2,3-bis(hexadecylthio)propan-1-ol (2C16S-Propargyl-OH)

Propargyl alcohol (0.250 mL, 4.29 mmol, 1.0 mol equivalent), 1-Hexadecane-thiol (3.33 grams, 12.88 mmol, 3 mol equivalents) and a solution of 0.24 M AIBN in toluene (540 μL, 0.13 mmol, 1 mol % with respect to the thiol) were dissolved and mixed in 30 mL EtOH in a Schlenk type of flask. The flask was sealed with rubber septum and then purged from oxygen by inserting a stainless-steel needle and bubbling Argon for 30 minutes. The flask was placed at 70° C. overnight with constant stirring.

The reaction mixture was allowed to cool down to room temperature and was quenched by opening the flask to air. The obtained solution was transferred to an evaporation flask and the EtOH was evaporated to dryness. The obtained crude 2C16S-Propargyl-OH product (an oily residue) was purified by dissolving the oily residue with DCM and loading the obtained solution on a silica column with 100% DCM as an initial eluent, following by 2.5% and then 5% MeOH in DCM as eluents, while monitoring by TLC using Hexanes:ethyl acetate (EtOAc) 90:10 v/v with I₂ staining (KMnO₄ can also be applied). The thiol and initiator had Rf values of 0.8 and 0.7, respectively, and the product showed Rf value of 0.4. Fractions that contained the product were combined, evaporated using rotary evaporator, and dried under high vacuum to afford 2.21 grams of a clear viscous oil in 90% yield. The product can be obtained as a white solid by re-dissolving the oily residue in DCM (5 mL per 1 gram) and adding hexanes (20 mL per 1 gram) and again evaporation and drying under high vacuum.

### Synthesis of 2,3-bis(hexadecylthio)propyl 2-bromo-2-methylpropanoate (2C16S-Propargyl-Br Initiator; Also Referred to Herein as 2C16S-Propargyl-Dm-Br Initiator or 2C16S-Prop-dm-Br Initiator)

2C16S-Propargyl-OH (0.958 gram, 1.67 mmol, 1 mol equivalent) and pyridine (0.162 mL, 2.00 mmol, 1.2 mol equivalent) were dissolved in a single neck round-bottomed flask using 10 mL DCM. The flask was cooled using an ice-bath and BIBB (0.25 mL, 2.00 mmol, 1.2 mol equivalent) was added slowly using a syringe. The reaction mixture was stirred for 2 hours in while reaching to ambient temperature.

The obtained reaction mixture was filtered-off gravitationally using common filtration paper. The flask and the filtered solids were washed with DCM (2×25 mL). The clear filtrate was evaporated to dryness using rotary evaporator. The obtained crude mixture was re-dissolved in Hexanes: EtOAc 90:10 v/v and loaded on a silica column using the same solvent mixture as eluent. The 2C16S-Propargyl-dm-Br product eluted first and was identified with TLC (eluent is Hexanes: EtOAc 90:10 v/v, Rf of 0.75) using I₂ or KMnO₄ for staining. Fractions that contained the purified product were combined, evaporated to dryness using rotary evaporator, and dried under high vacuum to give 1.19 gram of the 2C16S-Propargyl-dm-Br product as a clear oily residue in quantitative yield.

Synthesis of 2C16S-Prop-dm-pMPC (also referred to herein as 2C16S-Prop-pMPC) via ARGET-ATRP was performed as described for the preparation of DPPE-Ph-pMPC by replacing the DPPE-Ph-Br initiator with 2C16S-Prop-dm-Br.

### Preparation of 2C16-TMP-Dm-Br Initiator (Also Referred to Herein as 2C16-TMP-Br)

The exemplary 2C16-TMP-dm-Br was prepared in a two-step process from a trimethylolpropane (TMP) as a highly symmetrical core branching unit. By reacting TMP with BIBB, 2×OH-TMP-dm-Br was obtained and then reacted with palmitoyl chloride to form the 2C16-TMP-dm-Br initiator, as described herein and as depicted schematically in FIG. 6A.

### Synthesis of 2,2-bis(hydroxymethyl)butyl 2-bromo-2-methylpropanoate (2×OH-TMP-dm-Br; Also Referred to Herein as 2×OH-TMP-Br)

TMP (6.38 grams, 47.6 mmol, 3 mol equivalents) and pyridine (1.4 mL, 17.4 mmol, 1.1 mol equivalent) were dissolved in a single neck round-bottomed flask using THF (10 mL per 1 gram of TMP). The flask was cooled to about 5° C. using an ice bath. BIBB (2.0 mL, 15.9 mmol, 1 mol equivalent) was diluted with THF (5 mL per 1 mL of BIBB) and loaded into a dropping funnel. Then, the BIBB solution was added dropwise to the reaction flask. Once addition was over, the ice-bath was removed, and the reaction was allowed to stir for 2 more hours while reaching ambient temperature.

The obtained reaction was filtered-off gravitationally using common filtration paper. The reaction flask and the filtered solids were washed with EtOAc (2×25 mL). The clear combined organic filtrate was transferred to a separation funnel and further diluted using EtOAc (40 mL per 1 gram of TMP). The organic phase was washed twice with equivalent volumes of water, twice with equivalent volumes of 1 M HCl and finally once with an equivalent volume of brine. Then, the organic phase was dried using anhydrous MgSO₄, filtered, and evaporated to dryness. The obtained crude product was further purified by re-dissolving it with DCM and loading it on a silica column using 75:25 v/v EtOAc: Hexanes as eluent. Product was identified using TLC (eluent is Hexanes: EtOAc 1:1 v/v, Rf=0.4) with I₂ or KMnO+staining. Fractions containing the pure product were unified, evaporated to dryness using rotary evaporator, and dried under high vacuum to give 3.72 grams of the 2×OH-TMP-Br product as clear viscous oil in 83% yield.

### Synthesis of 2-(((2-bromo-2-methylpropanoyl)oxy)methyl)-2-ethylpropane-1,3-diyl dipalmitate (2C16-TMP-dm-Br Initiator)

2×OH-TMP-Br (3.08 grams, 10.88 mmol, 1 mol equivalent) and pyridine (1.9 mL, 24 mmol, 2.2 mol equivalents) were dissolved in DCM (10 mL per 1 gram of 2×OH-TMP-Br) in a round-bottomed single neck flask. The flask was cooled to about 5° C. using an ice-bath. Palmitoyl chloride (7.3 mL, 24 mmol, 2.2 mol equivalents) was diluted in DCM (5 mL per 1 mL of palmitoyl chloride) and loaded into a dropping funnel. The Palmitoyl chloride DCM solution was added dropwise to the reaction flask. Once addition was over, the ice-bath was removed, and the reaction was allowed to stir for 2 more hours and reach ambient temperature.

The reaction mixture was filtered-off gravitationally using common filter paper. The flask and the filtered solids were washed with DCM (2×25 mL). The clear filtrate was evaporated to dryness using rotary evaporator and the obtained crude mixture was re-dissolved with Hexanes: EtOAc 90:10 v/v and loaded on a silica column with the same composition of solvents as eluent. The product was identified using TLC (eluent is the same as the column, Rf=0.5) using 12 or KMnO₄ staining. Fractions containing pure product were unified, evaporated to dryness, and placed under high vacuum. The 2C16-TMP-dm-Br product was obtained as a clear viscous oil and upon overnight drying under high vacuum it solidified into a waxy white solid to give 7.21 grams of 2C16-TMP-dm-Br in 87% yield.

Synthesis of 2C16-TMP-dm-pMPC and 2C16S-Prop-dm-pMPC via ARGET-ATRP was performed as described for the preparation of DPPE-Ph-pMPC (Procedure 2) by replacing the DPPE-Ph-Br initiator with 2C16-TMP-dm-Br and 2C16S-Prop-dm-Br, respectively.

A summary of the obtained LPCs prepared based on either Procedure 1, 2 or 3, for each exemplary initiator, is presented in the following Table 3.

TABLE 3

| | | | | | | Obtained |
|---|---|---|---|---|---|---|
| | | Mol | Based on | Reaction | | DPn |
| | | ratio | Procedure | duration | | (#MPC |
| # | Lipid initiator | MPC:initiator | # | (hour) | LPC product | units) |
| 1 | 2C16-TMP- | 30 | 2 | 15-20 | 2C16-TMP- | 40-60 |
| | dm-Br | | | | dm-pMPC_S | |
| 2 | | 60 | | | 2C16-TMP- | 80-120 |
| | | | | | dm-pMPC_L | |
| 3 | 2C16S-Prop- | 30 | | | 2C16S-Prop- | 40-60 |
| | dm-Br | | | | dm-pMPC_S | |
| 4 | | 60 | | | 2C16S-Prop- | 80-120 |
| | | | | | dm-pMPC_L | |
| 5 | DPPE-dm-Br | 30 | | | DPPE-dm- | 180-200 |
| | | | | | pMPC_L | |
| 6 | | 8 | 1 | | LS1 | 50-80 |
| 7 | DPPE-Ph-Br | 25 | 2 | 20-22 | DPPE-Ph- | 50-80 |
| 8 | | 30 | | | pMPC_S | |
| 9 | | 80 | | | DPPE-Ph- | 80-120 |
| | | | | | pMPC_L | |
| 10 | | 30 | 3 | 6-8 | DPPE-Ph- | 50-80 |
| 11 | | | | | pMPC_S | |
| 12 | | | | | | |
| 13 | | | | | | |

### Example 5

### Liposomes Comprising the Newly Designed LPCs

The newly designed LPCs were used for preparing liposomes comprising different concentrations of the LPCs.

The initiators DPPE-Ph-Br, 2C16S-Propargyl-Br, and 2C16-TMP-Br were synthesized using ARGET-ATRP as described herein and then used to prepare exemplary LPCs having a MPC chain length of 30-80 (e.g., 50-80)(referred to herein as “short” or “S”) or 80-120 (referred to herein as “long” or “L”) units per initiator molecule. 0.7% or 3.5% of each LPC were used in the formulation of the liposomes.

In brief, batches with a polymer concentration of 0.7% were produced according to ethanol injection technique [Lombardo et al. Pharmaceutics, 2022, 14, 543]. An ethanol mixture containing DSPC and LPC was prepared at a mol ratio of 15:0.1. Solvent was removed by rotary evaporation to form a thin lipid film. Then, the obtained film was dissolved in ethanol in the amount of 10% of the target volume at 65° C. Multilamellar vesicles (MLVs) are formed by injection of the lipid solution directly into hot Phosphate-buffered saline (PBS). Hydration was carried out for at least 1 hour at T=65° C. with constant stirring. MLVs were downsized by extrusion using high pressure to obtain small unilamellar vesicles (SUVs) with an average size of 170 nm. Tangential flow filtration (TFF) system with hollow fiber column (molecular cut-off of 100 kDa) was used for ethanol removal. Liposome solution, previously diluted to the required concentration, was packaged in glass vials and steam-sterilized at T=121° C.

Formulations with 3.5% polymer were prepared according to the thin-film hydration method [Lomnardo, 2022, supra]. DSPC and LPC at a mol ratio of 48:1.8 were mixed and dissolved in ethanol, then the solvent was evaporated to obtain a thin lipid film. Lipid cake was dried under vacuum overnight followed by dispersion in PBS for at least 1 hour at 65° C. SUVs with the size of 170 nm were prepared by the extrusion method. Final liposome solution was packaged and steam-sterilized at T=121° C.

The correspondence between the used polymer, abbreviation, batch number of liposome sample, and LPC content in the composition is shown in Table 4 below.

TABLE 4

| LPC | LPC type | Batch | % LPC |
|---|---|---|---|
| DPPE-dm-pMPC_S | DM_S | IM22-669 | 0.7 |
| DPPE-Ph-pMPC_S | Ph_S | IM22-672 | |
| DPPE-Ph-pMPC_L | Ph_L | IM22-687 | |
| 2C16S-Prop-pMPC_S | Prop_S | IM22-674 | |
| 2C16S-Prop-pMPC_L | Prop_L | IM22-686 | |
| 2C16-TMP-pMPC_S | TMP_S | IM22-679 | |
| 2C16-TMP-pMP_L | TMP_L | IM22-682 | |
| DPPE-dm-pMPC_S | DM_S | IM22-675 | 3.5 |
| DPPE-Ph-pMPC_S | Ph_S | IM22-676 | |
| DPPE-Ph-pMPC_L | Ph_L | IM22-683 | |
| 2C16S-Prop-pMPC_S | Prop_S | IM22-677 | |
| 2C16S-Prop-pMPC_L | Prop_L | IM22-684 | |
| 2C16-TMP-pMPC_S | TMP_S | IM22-680 | |
| 2C16-TMP-pMP_L | TMP_L | IM22-682 | |

DPPE-dm-pMPC_S is polymerized using ATRP polymerization followed by separation using precipitation and silica column, as described herein (based on Procedure 1).

Distearoylphosphatidylcholine (DSPC) assay by HPLC was performed by dilution of the liposome sample×100 followed by injection of the sample to a Kinetex C8 column equilibrated to 40° C. Lipid components were eluted by a gradient using two solvents for the mobile phase: Solvent A—H₂O:MeOH 95:5 v/v and solvent B—100% MeOH. Gradient used was from 52.6% solvent A+47.4% solvent B to 100% solvent B. A charged Aerosol Detector (CAD) was used for detection of the eluting materials.

1-Stearoyl-2-hydroxy-sn-glycero-3-phosphocholine (18:0 LysoPC, LSPC) and stearic acid (SA) assay by HPLC was performed by dilution of the liposome sample ×20 followed by injection of the sample to a Gemini C18 column equilibrated to 50° C. lipid components were eluted by a gradient using two solvents for the mobile phase: Solvent A—H₂O:MeOH 95:5 v/v, 20 mM Ammonium Acetate and solvent B—100% MeOH, 20 mM Ammonium Acetate. Gradient used was from 52.6% solvent A+47.4% solvent B to 100% solvent B. A charged Aerosol Detector (CAD) was used for detection of the eluting materials.

The resulting liposomes were studied by HPLC, DLS, DSC, and CryoTEM, as described herein. HPLC analyses were performed in different time intervals, up to two months. The results are summarized in Table 5 below.

TABLE 5

| | | | | | After 2 weeks at | | | After a month at | | | | | |
|---|---|---|---|---|---|---|---|---|---|---|---|---|---|
| | | | | | 40° C. (1.5 | | | 40° C. (3.5 | | | After two | | |
| | | Zero-point | | | months at RT) | | | months at RT) | | | months at RT | | |
| | LPC | DSPC | LSPC | SA | DSPC | LSPC | SA | DSPC | LSPC | SA | DSPC | LSPC | SA |
| Batch | type | % | % | % | % | % | % | % | % | % | % | % | % |

0.7% polymer

| | | | | | | | | | | | | | |
|---|---|---|---|---|---|---|---|---|---|---|---|---|---|
| IM22-669 | DM_S | 99 | 4.5 | 4.7 | — | — | — | — | — | — | 100 | 6.4 | 6.1 |
| IM22-672 | Ph_S | 81 | 4.7 | 5.1 | 77 | 7.4 | 7.0 | 77 | 9.6 | 8.7 | — | — | — |
| IM22-687 | Ph_L | 73 | 4.4 | 4.6 | 72 | 7.1 | 6.8 | — | — | — | — | — | — |
| IM22-674 | Prop_S | 83 | 4.9 | 5.4 | 78 | 7.3 | 7.0 | 83 | 9.1 | 8.3 | — | — | — |
| IM22-686 | Prop_L | 91 | 4.8 | 5.2 | 90 | 7.2 | 6.8 | 89 | 9.7 | 8.8 | — | — | — |
| IM22-679 | TMP_S | 88 | 4.9 | 4.5 | 82 | 7.6 | 6.8 | 86 | 9.6 | 8.6 | — | — | — |
| IM22-682 | TMP_L | 81 | 4.9 | 5.3 | 79 | 7.2 | 6.8 | 81 | 9.4 | 8.4 | — | — | — |

3.5% polymer

| | | | | | | | | | | | | | |
|---|---|---|---|---|---|---|---|---|---|---|---|---|---|
| IM22-675 | DM_S | — | — | — | — | — | — | — | — | — | 88 | 2.1 | 2.0 |
| IM22-676 | Ph_S | — | — | — | — | — | — | — | — | — | 91 | 2.3 | 2.0 |
| IM22-683 | Ph_L | — | — | — | — | — | — | — | — | — | 85 | 2.0 | 1.8 |
| IM22-684 | Prop_L | — | — | — | — | — | — | — | — | — | 89 | 2.6 | 2.1 |
| IM22-680 | TMP_S | — | — | — | — | — | — | — | — | — | 91 | 2.6 | 2.5 |
| IM22-681 | TMP_L | — | — | — | — | — | — | — | — | — | 77 | 2.7 | 2.3 |

As can be seen from these data, no significant difference in DSPC assay and concentration 5 of degradation products over the course of two or more months was observed for any of the liposomes with 0.7% LPC additive. When using 3.5% LPCs, some degradation products were detected. Without being bound to any particular theory, it is assumed that the minor degradation results from decreased hydrolysis rate caused by the dense polymer layer on the liposome surface.

Next, size distribution was performed by DLS in different time intervals, up to two months, and the results are summarized in Table 6 (0.7% LPC) and Table 7 (3.5% LPC) below. 10

TABLE 6

| | | Batch | | | | | | |
|---|---|---|---|---|---|---|---|---|
| | | IM22- | IM22- | IM22- | IM22- | IM22- | IM22- | IM22- |
| | | 669 | 672 | 687 | 674 | 686 | 679 | 682 |
| | | LPC type | | | | | | |
| | | DM_S | Ph_S | Ph_L | Prop_S | Prop_L | TMP_S | TMP_L |
| | | % LPC | | | | | | |
| Conditions | | 0.7% | | | | | | |
| Zero-point | Z-avg | 156 | 161 | 172 | 175 | 162 | 158 | 167 |
| | PDI | 0.073 | 0.063 | 0.071 | 0.083 | 0.066 | 0.105 | 0.063 |
| | Peak | 170 | 173 | 186 | 190 | 171 | 176 | 181 |
| | Osm | 284 | 285 | 277 | 278 | 286 | 284 | 282 |
| | pH | 7.48 | 7.51 | 7.51 | 7.52 | 7.4 | 7.46 | 7.48 |
| After a week | Z-avg | — | 157 | 168 | 173 | 161 | 156 | 166 |
| at RT | PDI | — | 0.096 | 0.06 | 0.12 | 0.067 | 0.094 | 0.054 |
| | Peak | — | 174 | 180 | 196 | 174 | 164 | 178 |
| After two | Z-avg | — | 162 | 174 | 173 | 164 | 157 | 169 |
| weeks at | PDI | — | 0.080 | 0.075 | 0.097 | 0.064 | 0.093 | 0.07 |
| 40° C. (~1.5 | Peak | — | 177 | 189 | 192 | 177 | 174 | 184 |
| months at | Osm | — | 282 | 282 | 291 | 282 | 286 | 282 |
| RT) | pH | — | 7.36 | 7.35 | 7.37 | 7.33 | 7.35 | 7.33 |
| After one | Z-avg | — | 165 | — | 174 | 164 | 160 | 169 |
| month at | PDI | — | 0.082 | — | 0.099 | 0.103 | 0.091 | 0.067 |
| 40° C. (~3.5 | Peak | — | 181 | — | 195 | 184 | 177 | 182 |
| months at | Osm | — | 282 | — | 291 | 282 | 280 | 283 |
| RT) | pH | — | 7.49 | — | 7.39 | 7.36 | 7.38 | 7.36 |
| After two | Z-avg | 154 | — | — | — | — | — | — |
| months at RT | PDI | 0.091 | — | — | — | — | — | — |
| | Peak | 171 | — | — | — | — | — | — |
| | Osm | 279 | — | — | — | — | — | — |
| | pH | 7.54 | — | — | — | — | — | — |

TABLE 7

| | | Batch | | | | | | |
|---|---|---|---|---|---|---|---|---|
| | | IM22- | IM22- | IM22- | IM22- | IM22- | IM22- | IM22- |
| | | 675 | 676 | 683 | 677 | 684 | 680 | 681 |
| | | LPC type | | | | | | |
| | | DM_S | Ph_S | Ph_L | Prop_S | Prop_L | TMP_S | TMP_L |
| | | % LPC | | | | | | |
| Conditions | | 3.5% | | | | | | |
| Zero-point | Z-avg | 167 | 165 | 173 | 161 | 170 | 171 | 167 |
| | PDI | 0.072 | 0.07 | 0.042 | 0.08 | 0.085 | 0.081 | 0.082 |
| | Peak | 181 | 179 | 183 | 177 | 186 | 188 | 182 |
| | Osm | 347 | 314 | 337 | 364 | 347 | 362 | 340 |
| After a week | Z-avg | 169 | 167 | 172 | 166 | 173 | 173 | 170 |
| at room | PDI | 0.081 | 0.067 | 0.056 | 0.071 | 0.071 | 0.1 | 0.068 |
| temperature | Peak | 185 | 181 | 184 | 179 | 189 | 193 | 184 |
| After two | Z-avg | 174 | 170 | 175 | 169 | 174 | 176 | 171 |
| months at | PDI | 0.074 | 0.074 | 0.09 | 0.064 | 0.085 | 0.094 | 0.074 |
| room | Peak | 189 | 185 | 192 | 184 | 191 | 195 | 186 |
| temperature | Osm | 346 | 314 | 335 | 364 | 347 | 361 | 356 |
| | pH | 7.31 | 7.29 | 7.24 | 7.34 | 7.29 | 7.32 | 7.32 |

As can be seen, the size of the liposomes was not significantly affected even after 2 months at room temperature, which indicates they remained stable at these conditions.

Next. Z-potential of the obtained liposomes was measured by DLS and the results are summarized in Table 8 below.

TABLE 8

| | LPC | % | Z-potential | Conductivity | DCR | |
|---|---|---|---|---|---|---|
| Batch | type | LPC | [mV] | [mS/cm] | [kcps] | Attenuator |
| IM22-669 | DM_S | 0.7 | −21.10 | 0.24 | 5883 | 8 |
| IM22-675 | | 3.5 | −15.37 | 0.41 | 8525 | 7 |
| IM22-675 | | 3.5 | −20.13 | 0.23 | 5335 | 8 |
| (diluted) | | | | | | |
| IM22-672 | DPPE-Ph- | 0.7 | −15.97 | 0.22 | 6429 | 8 |
| IM22-676 | pMPC_S | 3.5 | −19.43 | 0.42 | 12937 | 7 |
| IM22-687 | DPPE-Ph- | 0.7 | −12.47 | 0.22 | 6032 | 8 |
| IM22-683 | pMPC_L | 3.5 | −12.47 | 0.44 | 9962 | 7 |
| IM22-674 | 2C16S-Prop- | 0.7 | −12.97 | 0.22 | 10343 | 7 |
| IM22-677 | pMPC_S | 3.5 | −12.07 | 0.42 | 7172 | 7 |
| IM22-686 | 2C16S-Prop- | 0.7 | −8.08 | 0.22 | 7830 | 7 |
| IM22-684 | pMPC_L | 3.5 | −7.73 | 0.44 | 10226 | 7 |
| IM22-679 | 2C16-TMP- | 0.7 | −12.13 | 0.22 | 7134 | 8 |
| IM22-680 | pMPC_S | 3.5 | −10.63 | 0.43 | 11464 | 7 |
| IM22-682 | 2C16-TMP- | 0.7 | −9.18 | 0.24 | 4840 | 8 |
| IM22-681 | pMPC_L | 3.5 | −8.22 | 0.43 | 6205 | 8 |

No significant difference was found in Z-potential for pairs of batches with different concentrations of the same LPC. The conductivity of the sample affects the Z-potential, as can be observed for the DPPE-dm-pMPC_S-containing liposomes, after correcting the conductivity by diluting batch IM22-675, the Z-potential leveled off.

Without being bound to any particular theory, it is assumes that the charges of the MPC and DSPC are compensated by their zwitterion forms, while the DPPE fragment of batches IM22-669, IM22-672 and IM22-684 has a negative charge. Therefore, the total charge in the liposome solution should be negative. DPPE-Ph_S and DPPE-Ph_L, which possess a different polymeric chain length, a smaller negative charge was found with an increase in the molecular weight of the polymer. The weakening of the negative charge may be due to an increased distance from the DPPE charged fragment on the liposome surface. The absence of the DPPE fragment in the Propargyl- and TMP-based LPCs results in a lower Z-potential compared to the DPPE-based LPCs.

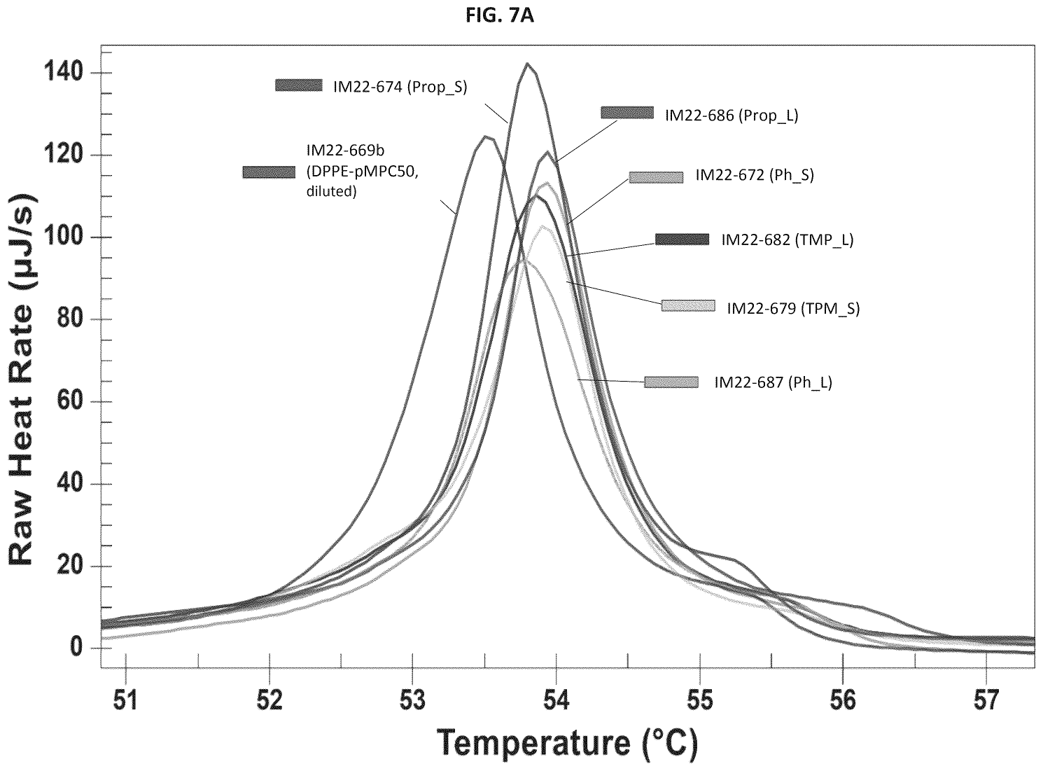

DSC analyses were performed. The obtained data is presented in FIGS. 7A-C and summarized in Table 9 below.

TABLE 9

| | | % | Tmax | ΔH | ΔS |
|---|---|---|---|---|---|
| Batch | LPC type | LPC | (° C.) | (kJ / mol) | (kJ/(mol · K)) |
| IM22-669 | DPPE-dm-pMPC_S | 0.7 | 53.53 ± 0.03 | 139349 ± 922 | 427 ± 3 |
| IM22-672 | DPPE-Ph_S | 0.7 | 53.94 ± 0 | 140286 ± 3281 | 429 ± 10 |
| IM22-676 | | 3.5 | 53.09 ± 0.02 | 106204 ± 2542 | 326 ± 8 |
| IM22-687 | DPPE-Ph_L | 0.7 | 53.76 ± 0.04 | 146062 ± 3165 | 447 ± 10 |
| IM22-683 | | 3.5 | 52.94 ± 0.01 | 121490 ± 807 | 373 ± 2 |
| IM22-674 | 2C16S-Prop_S | 0.7 | 53.8 ± 0 | 164852 ± 14287 | 504 ± 44 |
| IM22-686 | 2C16S-Prop_L | 0.7 | 53.94 ± 0 | 134287 ± 415 | 411 ± 1 |
| IM22-684 | | 3.5 | 53.41 ± 0.03 | 126960 ± 1698 | 389 ± 5 |
| IM22-679 | 2C16-TMP_S | 0.7 | 53.92 ± 0.03 | 132758 ± 1154 | 406 ± 4 |
| IM22-680 | | 3.5 | 53.09 ± 0.02 | 128466 ± 550 | 394 ± 2 |
| IM22-682 | 2C16-TMP_L | 0.7 | 53.86 ± 0 | 142027 ± 1068 | 353 ± 48 |
| IM22-681 | | 3.5 | 53.13 ± 0.04 | 109319 ± 2686 | 335 ± 8 |

As can be seen in FIG. 7A and Table 9, DPPE-dm-pMPC_S shows significantly lower Tm, suggesting it has decreased thermodynamic stability and thus introduces the greatest interference to the lipid membrane. Liposome comprising the Prop_S LPC sample show similar thermodynamic values. Liposomes comprising Ph_S or TMP_S LPC show the highest Tm (see, FIGS. 7B and 7C).

No clear correspondence between Tm value and molecular weight of polymer was found for each pair of LPC, however, Tm of Prop_L is higher than Tm of Prop_S. Additionally, the shape of the graph received for the propargyl sample is different, which can be a result of different structure formation (possible interactions between the LPC and the DSPC (interparticle) membrane or between liposomes due to attracting interactions (intraparticle).

For all samples, there is a consistent decrease of about 1.5% in Tm when LPC concentration increase from 0.7% to 3.5%. This is consistent with the introduction of a different and shorter lipid to the membrane that causes distortion/disturbance in the lipid membrane. The highest change in Tm (for DPPE-Ph_S) correlates with the highest change in enthalpy (ΔH) and entropy (ΔS). Both values decrease between 5-30% for all samples when LPC is increased.

As LPC stabilization mechanism is controlled by steric interactions which do not play a role in DSC measurements, the nature of the secondary peak and its significance to the stability of the liposomes needs to be elucidated.

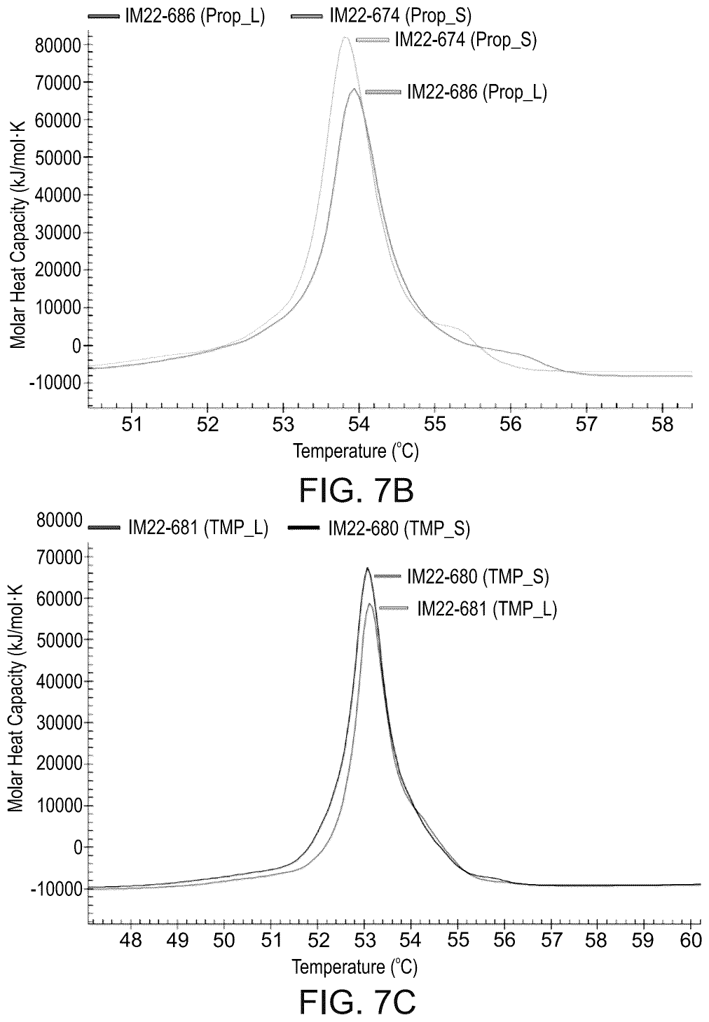

CryoTEM images are presented in FIGS. 8A-D. As can be seen, small unilamellar vesicles with a real membrane diameter of 100-200 nm were found in the tested samples. As FIG. 8A depicts, the sample comprising 3.5% of Ph_S LPC also formed multiple 30 nm micelles, which may be attributed to sample preparation. While each sample is characterized by a well-rounded shape, liposomes with TMP_L LPC have a somewhat distorted shape.

To conclude, liposomes comprising the exemplary LPCs (0.7% or 3.5% of DPPE-dm-pMPC, DPPE-Ph-pMPC, 2C16-Prop-pMPC, or 2C16-TMP-pMPC) having varying polymer chain lengths were prepared and analyzed. It was found that liposomes comprising even small amounts of such LPCs possess an average size of 100-200 nm, remain stable for at least 2 months at room temperature, and maintain stability at elevated temperatures of up to 50° C.

### Example 6

### In-Vitro Cytotoxicity Study

A biocompatibility study of the liposomes was performed for the evaluation of cytotoxicity.

A series of liposome samples containing the newly designed exemplary LPCs were compared with batches containing DPPE-dm-pMPC_S after one-year storage at room temperature.

Cytotoxicity test was performed on L929 mouse cell line (obtained from European Collection of Authenticated Cell Cultures (ECACC), Cat. N 85103115, NCTC clone 929, clone of L strain, mouse connective tissue). Cells were incubated with each liposome sample previously diluted up to 2 mM using L929 growth medium ×2.5 and water for tissue culture at 37±1° C., humidified 5±0.5% CO₂/air for 72±2 hours. Cells viability was determined by XTT labeling. For detection, each liposome sample was incubated with XTT/PMS mixture for approximately 3.5 hours, at 37±1° C., humidified 5±0.5% CO₂/air protected from light. The Cytotoxic potential of each tested sample was evaluated by the measurement of absorbance signals at 450 nm and 650 nm.

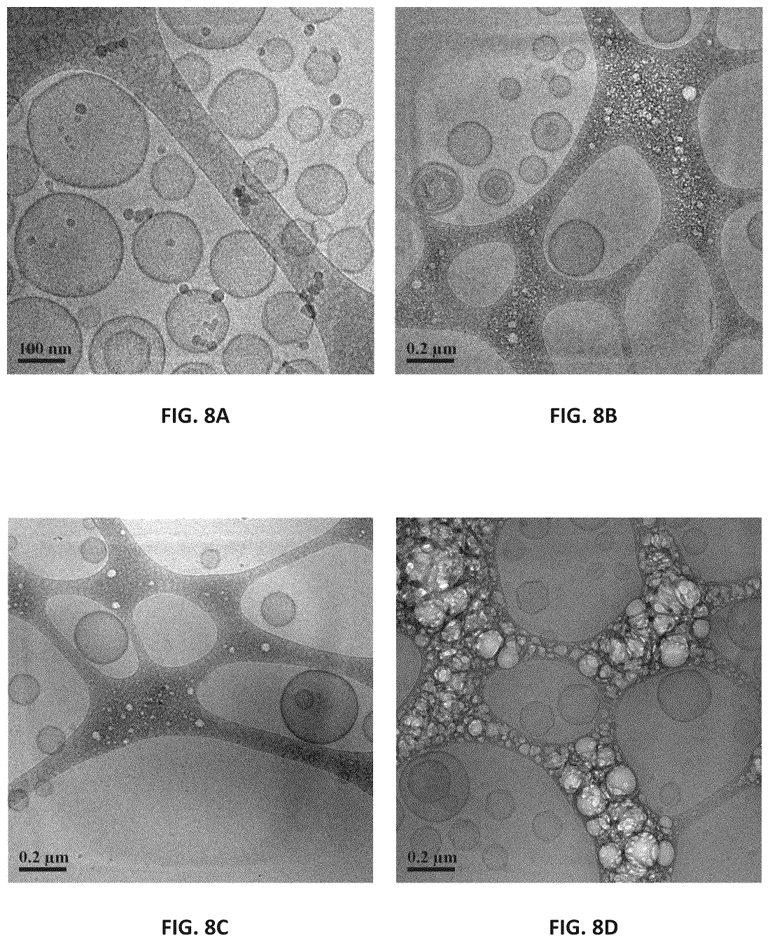

The obtained data is presented in FIG. 9 and show that all samples from the newly-designed polymer initiator series showed minor cytotoxic activity, as did the one-year stored batches with 0.7% of DM_S.

These data suggest that the liposomes remain stable and biocompatible even after prolonged storage of at least one year at room temperature.

### Example 7

### Immunogenicity

As is well-known in the art, lipidic nanoparticles such as liposomes can interact with the immune system and be detected as harmful particles, hence inducing hypersensitivity reactions (HSRs) or an infusion reaction. Such complement activation related pseudoallergy (CARPA) are considered a safety issue in nanopharmacotherapy as they can lead to potentially fatal outcome. CARPAgenic activity occurs directly upon first exposure to lipid excipients, including lipid nanoparticles, without prior sensitization. Symptoms usually decrease or disappear on subsequent treatment. Patients sometimes require short infusions of placebo drug-free liposomal compounds, to avert hypersensitivity.

Dominating factors that trigger CARPA reactions are liposomal size, morphology, lamellarity and in particular the liposomal surface charge which is often modulated by incorporation of charged lipids to achieve the intended therapeutic effects [see, Marwa et al., Science and Technology of Advanced Materials, 2019, 20 (1), 710-724].

Complement (C) activation can be measured in vitro by measuring the C cleavage products in human sera or in vivo in relevant animal models, with the pig being the most sensitive model. Animal studies of CARPA have demonstrated the major impact of liposomal surface characteristics which determines the presence or absence of tachyphylaxis. See, for example, Dezsi et al. J Control Release. 2014, 195, 2-10.

It was shown that large multilamellar vesicles composed of DSPG led to stronger vasoactive consequences compared the uncharged DMPC liposome, highlighting the importance of charge influence on complement activation. See, for example, Alinaghi et al., Int J Pharm. 2014, 459 (1-2), 30-39; Szebeni J. Crit Rev Ther Drug Carrier Syst. 1998, 15 (1), 57-88.

A well-known route to lower immunogenicity is the surface modification of the liposomes with hydrophilic polymers such as polyethylene glycol (PEG). Steric hindrance effects based on the polymeric corona protect the liposomes from immune system recognition. Yet, several studies report low efficiency of shielding certain PEGylated liposomes from the immune system.

PEGylated DPPC liposomes with incorporated methoxy PEG, which contains a negatively charged phosphor diester moiety that is grafted to DSPE, results in a substantial complement activation. The CARPAgenic activity was reduced by methylation of the acidic moiety and the reduction in addition proven by non mPEGylated liposomes. Furthermore, it has been demonstrated that PEGylated liposomes with negative zeta potentials, including Doxil®, activate the complement system.

Considering the limitations of PEGylated lipids to avoid induction of HSRs, there is a need to develop more efficient stealth polymers.

The effectiveness of polymeric moieties in stabilizing liposomes is correlated with the degree of the liposome surface coverage by the polymer. A general method to determine the degree of surface coating and the coating layer thickness employs zeta potential measurements of charged liposomes incorporating increasing amounts of grafted polymer.

The present inventors have thus turned to determine the optimal surface coverage of liposomes by the LPC of the present embodiments, using zeta potential measurements, in particular for liposomes composed of DSPC/DPPG/LPC at a fixed DPPG concentration of 10 mol %, LPC in the range of 0-6 mol %, and DSPC in the range of 84-90 mol %, depending on the specific LPC concentration, to complement to a 100 mol % of total lipids.

To evaluate the degree of non-immunogenicity properties of the LPCylated liposomes, CARPA activity in whole blood human sera in the presence of the pMPCylated liposomes was measured in-vitro using an ELISA. Incorporating cholesterol as a component is also tested for CARPA.

### Materials and Methods

Particles size was evaluated by dynamic light scattering (DLS) measurements using a Zetasizer Nano instrument by Malvern Panalytical. Liposome samples were diluted in PBS to a final volume concentration of 1 mM, transferred to a disposable polystyrene cuvette and equilibrated to 25° C., followed by three replicate measurements. The mean Z-average diameter and mean polydispersity index (PDI) were reported.

Zeta-Potential was determined using a Zetasizer Nano instrument by Malvern Panalytical. Samples were diluted×100 to a final concentration of 10 μM in 10 mM HEPES pH 5.7, transferred to a disposable folded capillary cuvette and equilibrated to 25° C., followed by three replicate measurements.

Liposome-induced C-activation was estimated by measuring the formation of S protein-bound C-terminal complex (SC5b-9) with an enzyme-linked immunosorbent assay (ELISA). Samples were diluted 4 times with serum, incubated at 37° C. for 45 minutes, and then terminated by adding kit Specimen Diluent supplemented with 20 mM EDTA. PBS was used as negative control, Zymosan—positive control.

Liposomes preparation was performed by the thin-film hydration method. Depending on the specific formulation, phospholipids (1,2-distearoyl-sn-glycero-3-phosphocholine; DSPC and/or 1,2-dipalmitoyl-sn-glycero-3-phosphoglycerol; DPPG), polymer (DSPE-dm-pMPC or DPPE-dm-pMPC or DPPE-Ph-pMPC, prepared in accordance with the exemplary procedures described hereinabove) and Cholesterol were mixed in appropriate ratios (according to Table 10) and dissolved in ethanol at 65° C. Ethanol was evaporated using rotary evaporator and the film was dried in the desiccator under vacuum overnight. The thin lipid layer was dispersed in PBS at 65° C. for at least one hour. The obtained particles were downsized by high-pressure extrusion, followed by packing and steam sterilization at 121° C.

### Results

### Liposome Composition and Size:

Formulations were separated to three groups, as shown also in Table 10:
1) formulations with DPPE-dm-pMPC and DSPE-dm-pMPC:
DSPE-dm: DSPC/DSPE-dm-pMPC (99.5:0.5 mol %)
DPPE-dm: DSPC/DPPE-dm-pMPC (99.5:0.5 mol %)
DPPE-Ph_S: DSPC/DPPE-Ph-pMPC (99.3:0.7 mol %)
2) negatively charged PC/PG formulations incorporating a “short” LPC (DPn=50) at 0-6 mol % of total lipids:
DPPG+0-6% Ph_S: DSPC/DPPG/DPPE-Ph-pMPC (90-84/10/0-6 mol %),
Mw(LPC)=17.2 kDa
3) negatively charged PC/PG formulations incorporating a “long” LPC (DPn=132) at 0-6 mol % of total lipids:
DPPG+0-6% Ph_L: DSPC/DPPG/DPPE-Ph-pMPC (90-84/10/0-6 mol %),
Mw(LPC)=39.8 kDa

TABLE 10

| | | Total lipid, | Z-average, | |
|---|---|---|---|---|
| N | Sample | mM | nm | PDI |
| 2 | DSPE_dm | 6.4 | 156 | 0.08 |
| 3 | DPPE_dm | 6.4 | 165 | 0.09 |
| 4 | DPPE_Ph_S | 10 | 161 | 0.06 |
| 5 | DPPG + 0% Ph_S | 10 | 182 | 0.10 |
| 6 | DPPG + 0.7% Ph_S | | 170 | 0.08 |
| 7 | DPPG + 1.5% Ph_S | | 163 | 0.08 |
| 8 | DPPG + 3% Ph_S | | 161 | 0.08 |
| 9 | DPPG + 6% Ph_S | | 157 | 0.10 |
| 10 | DPPG + 0% Ph_L | 10 | 178 | 0.09 |
| 11 | DPPG + 0.7% Ph_L | | 170 | 0.09 |
| 12 | DPPG + 1.5% Ph_L | | 178 | 0.07 |
| 13 | DPPG + 3% Ph_L | | 176 | 0.09 |
| 14 | DPPG + 6% Ph_L | | 166 | 0.07 |

### Effect of Liposome Charge and Polymer Length on C-Activation:

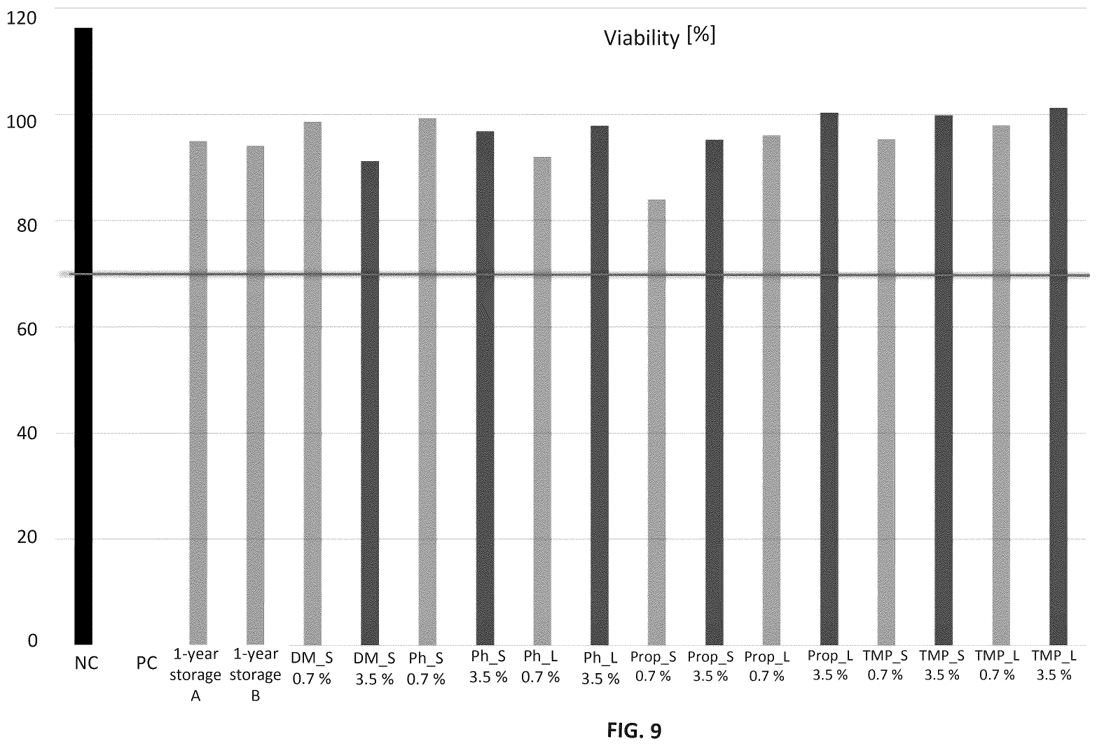

In a first tested series, three types of polymers were tested: (1) DM_S with DSPE as the hydrophobic anchor and (2) DM_S with DPPE as the hydrophobic anchor and (3) Ph_S with a DPPE lipid modified with a phenyl group. DM polymers with different anchors were prepared according to the same synthetic procedure and have approximately close molecular weight (14-18 kDa). The data is presented in FIG. 10A and show that a sufficient decrease of HSRs (almost 2-fold) was obtained for formulation containing a DPPE-Ph-pMPC polymer structure.

To elucidate the capacity of LPC-containing liposomes to reduce immunogenic responses, two series of liposomes incorporating LPC at a range of 0-6 mol % concentrations with negative charged DPPG lipid were tested, each with a different polymer length, and the results are presented in FIG. 10B. As can be seen, the CARPA response reduces with increasing LPC membrane content. Further, liposomes incorporating the long LPC were more effective in reducing CARPA reaction, especially compositions with 3 and 6% of LPC (CARPA values are close to the PBS values).

These data demonstrate that LPC suppresses charge related CARPA reactions and is highly effective in masking the liposomes from C-activation factors.

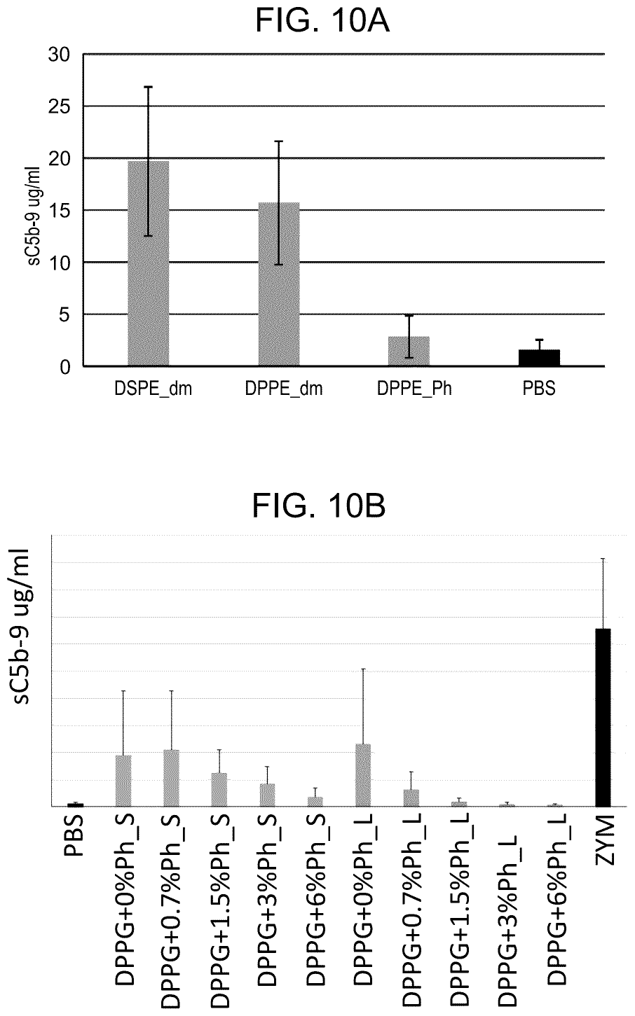

The immunogenicity of the liposomes incorporating the long LPC are highly correlated with Zeta potential of these formulations, shown in FIG. 11, further supporting that LPC is an efficient steric barrier capable of masking immunogenic factors related to liposome surface (curvature, charge, and composition).

### Screening for Optimal pH for Zeta Potential Measurements of LPC:

An underlying requirement for evaluation of the masking effect of LPC on liposome surface charge is that the charge should originate from the surface only and not from the polymer. Since the repeating unit in LPC is ionizable and the pKa of polymers might be different than the pKa of the monomers composing it, the zeta potential of a water-soluble pMPC polymer, Ethyl-Phenyl-pMPC (DPn=45), was measured in the pH range of 5.34-7.66 in 10 mM Hepes buffer, at low ionic strength. This polymer is water-soluble and doesn't form micelles, therefore the zeta potential measured reflects the surface charge of individual polymers. The ionic strength was adjusted to 1±0.1 mS/cm by a 2 M NaCl stock solution. Ionic strength adjustment is necessary as the Zeta potential is very sensitive to the ionic strength. The dependence of the Zeta potential (ZP) on pH is depicted in FIG. 11. For Ethyl-Phenyl-pMPC, the ZP is slightly negative between pH 5.3-6.3, and decreases above pH 6.3. To demonstrate that the zeta potential for liposomes composed of zwitterionic lipids and LPC, originates from the LPC, the zeta potential of a DSPC/LPC 99.3/0.7 mol % incorporating LPC with a DPn of about 40 monomers was also measured. The ZP dependence of the liposomes behaves in a similar way, but with overall lower ZP values. This indicates that the ZP is contributed mainly from the MPC polymer. The slightly more negative values for the liposomes at pH<6.3 is due to contributions of the liposome lipids.

### Dependence of the Zeta Potential on LPC Length and Concentration in Low Salt Solutions:

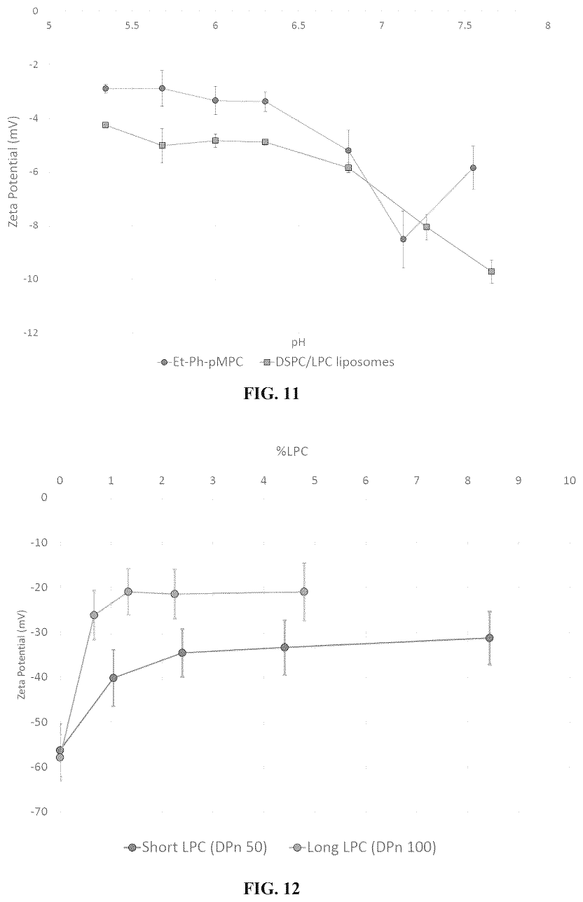

To determine the degree of surface coverage, the zeta potentials of the DSPC/DPPG/LPC liposome solutions were measured at pH 5.7 at low salt ionic strength and the obtained data is presented in FIG. 12. The efficiency of LPC to mask the liposome surface charge is both concentration- and LPC length-dependent. The capacity of the short LPCs to mask the surface charge is less than that of the long LPCs, as both the rate of ZP increase with LPC concentration and the final charge reduction is lower for the short LPC, as can be expected. The charge masking of the long LPC reaches full capacity at about 1.3 mol % while that of the short LPC at about 2.4 mol %. This is about 1.85-fold higher capacity for the long LPC, and it closely correlates to the difference in LPC mean length of about 2-fold.

The negative charge on the liposome surface is attributed to DPPG, which is considered to be distributed uniformly over the liposome membrane surfaces. Under these conditions the thickness of the LPC polymer layer on the liposome surface can be calculated. If zeta potentials are measured in various concentrations of NaCl, a plot of the Debye length κ=\sqrt{c}/0.3, where c is the total concentration of monovalent ions in solution, against ln(−ZP) can be fit to a linear equation. The slope of the fit gives the thickness of the fixed aqueous layer in nm units.

The total electrolyte concentration is contributed by buffer ions and NaCl. Hepes buffer has a pKa of 7.55 at 25° C. From the Henderson-Hasselbalch equation, about 1.1% of HEPES base will be dissociated at pH 5.7. Therefore, electrolyte concentration C was regarded as NaCl concentration plus 0.00011 M.

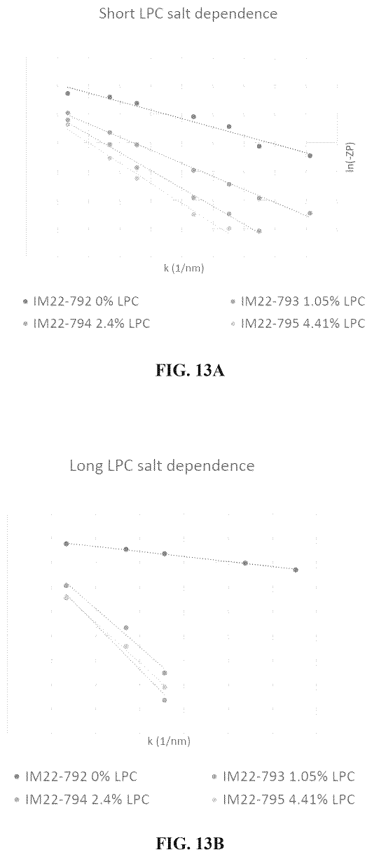

FIGS. 13A-B present comparative plots showing the dependence of ZP on electrolyte concentration. The effect of increasing salt concentration is lower for the short LPC than for the long LPC. This can be interpreted as resulting by lower charge screening capacity of the short LPC, requiring higher salt concentration to suppress the surface charge, whereas for the long LPC, masking efficiency is higher, and therefore lower salt is required to suppress the residual charge not masked by LPC.

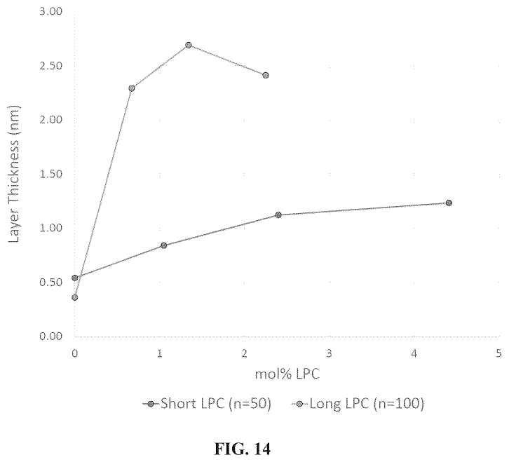

LPC layer thickness for both types of LPC is depicted in FIG. 14. The layer thickness for the long LPC at high LPC concentrations is about 3-fold higher compared to the short LPC (2 nm vs. 0.69 nm).

LPC Stealth Properties: Correlating Liposome Surface Properties with Non-Immunogenic Reactions:

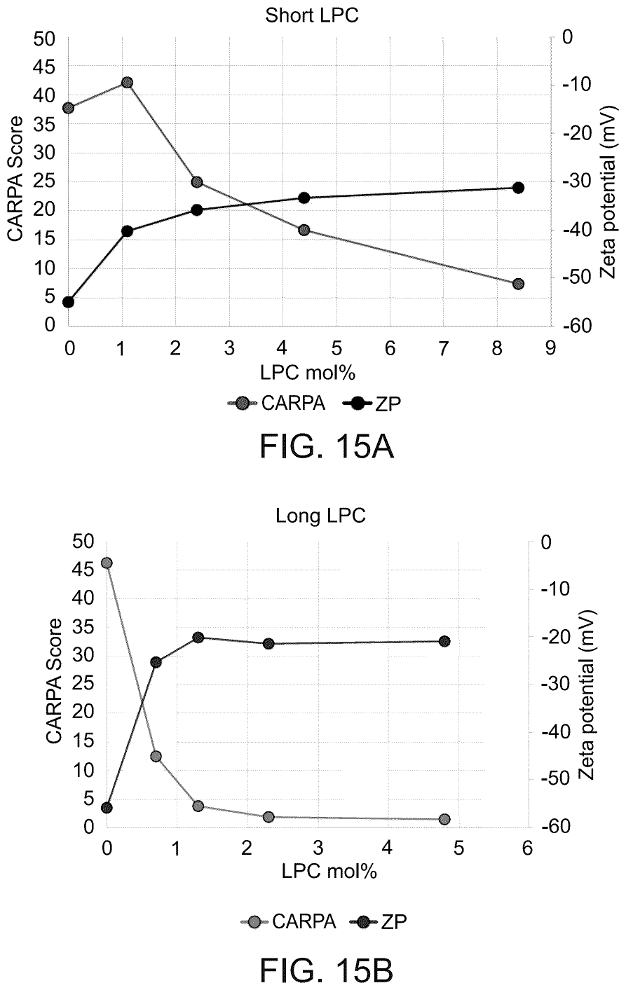

Evaluation of the CARPA results to the zeta potential reveals a clear correlation between the degree of surface coating and the capacity of LPCylated liposomes to avoid CARPA activation, as shown in FIGS. 15A-B.

Overall, the data presented in this Example suggest that for applications that require reduced immunogenicity, for example, for liposomes containing therapeutically active agents for drug delivery, or any other application that involves systemic administration of the liposomes, liposomes made of long LPCs as described herein are preferred and can be utilized also along with negatively charged lipids (which in other cases tend to increase immunogenic response). Inclusion of cholesterol in the lipid bilayer of such liposomes is also advantageous. Inclusion of negatively charged lipids in the liposomes is advantageous, for example, on cases where a positively charged therapeutically agent is included in (e.g., enveloped by) the liposome.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section 10 headings are used, they should not be construed as necessarily limiting. In addition, any priority document(s) of this application is/are hereby incorporated herein by reference in its/their entirety.

1. A polymeric compound represented by Formula I: 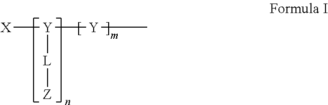
wherein:
m is zero or a positive integer;
n is an integer which is at least 2, at least 5, preferably at least 10 (e.g., of from 10 to 200);
Y is a backbone unit which forms a polymeric backbone of the polymeric compound;
L is absent or is a linking moiety; and
Z has the general Formula II: 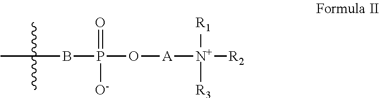
wherein:
the dashed (curved) line denotes an attachment point to the respective Y backbone unit or to the linking moiety L, if present;
A is a substituted or unsubstituted hydrocarbon;
B is an oxygen atom or is absent;
R₁-R₃ are each independently selected from the group consisting of hydrogen, alkyl, cycloalkyl, heteroalicyclic, aryl and heteroaryl;
X is a lipid moiety represented by Formula IV: 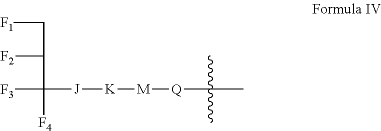
wherein:
the dashed (curved) line denotes an attachment point to said polymeric backbone;
F₁, F₂, F₃ and F₄ are each independently selected from the group consisting of hydrogen, alkyl, alkenyl, alkynyl, acyl, alkoxy, thioalkoxy, carboxy, thiocarboxy, wherein at least one of F₁, F₂, F₃ and F₄ is not hydrogen and is of at least 10 carbon atoms in length;
J is —O—P(═O)(OH)—O— or absent;
K is a substituted or unsubstituted hydrocarbon from 1 to 10 carbon atoms in length or absent;
M is a linking group selected from the group consisting of —O—, —S—, amino, sulfinyl, sulfonyl, phosphate, phosphonyl, phosphinyl, carbonyl, thiocarbonyl, urea, thiourea, carbamyl, thiocarbamyl, amido, carboxy, and sulfonamide, or absent; and
Q is a substituted or unsubstituted hydrocarbon from 1 to 10 carbon atoms in length, or absent,
wherein when M is absent, Q is also absent, and when J is absent, M is not absent,
provided that:
when J is —O—P(═O)(OH)—O—, M is other than amido and/or Q comprises an aryl moiety.

2. (canceled)

3. The polymeric compound of claim 1, wherein at least one of F₁, F₂, F₃ and F₄ is derived from a fatty acid selected from the group consisting of lauroyl, myristoyl, palmitoyl, stearoyl, palmitoleoyl, oleoyl, and linoleoyl.

4-5. (canceled)

6. The polymeric compound of claim 1, wherein J is —P(═O)(OH)—O—; M is amido; and Q is a hydrocarbon substituted by at least one aryl.

7. The polymeric compound of claim 1, wherein Q is a methylene substituted by at least one aryl.

8-9. (canceled)

10. The polymeric compound of claim 1, wherein J and K are each absent and M is carboxy.

11. The polymeric compound of claim 1, wherein at least one, or at least two, of F₁, F₂, F₃ and F₄ is independently said thioalkoxy and/or said carboxy.

12-13. (canceled)

14. The polymeric compound of claim 10, wherein Q is —C(CH₃)₂—.

15. The polymeric compound of claim 1, wherein Y is a substituted or unsubstituted alkylene unit.

16. (canceled)

17. The polymeric compound of claim 1, wherein:
B is an oxygen atom; and/or
A is a substituted or unsubstituted hydrocarbon from 1 to 4 carbon atoms in length; and/or
R₁-R₃ are each independently hydrogen or C₁₋₄-alkyl.

18-20. (canceled)

21. The polymeric compound of claim 1, wherein n is at least 30.

22. (canceled)

23. The polymeric compound of claim 1, wherein n is at least 50.

24-27. (canceled)

28. The polymeric compound of claim 1, wherein at least a portion of said backbone units Y, said L and/or said Z comprise at least one targeting moiety.

29. A lipid bilayer comprising at least one bilayer-forming lipid and the polymeric compound of claim 1.

30-32. (canceled)

33. A liposome comprising at least one lipid bilayer according to claim 29.

34. A composition comprising liposomes of claim 33 and a carrier, preferably an aqueous carrier.

35. The composition of claim 34, being a sterile composition and/or a lubricant composition.

36. (canceled)

37. The composition of claim 35, further comprising a water-soluble polymer.

38. (canceled)

39. A method of reducing a friction coefficient of a surface, the method comprising contacting the surface with a composition that comprises a liposome according to claim 33 and carrier.

40. (canceled)

41. The method of claim 39, further comprising contacting the surface with a water-soluble polymer.

42. The method of claim 39, wherein said surface is a physiological surface, and said carrier is a physiologically acceptable carrier.

43. The method of claim 42, wherein said surface is an articular surface of a synovial joint.

44. (canceled)

45. A method of inhibiting biofilm formation on a surface of a substrate, the method comprising contacting the substrate with a composition which comprises a liposome according to claim 33.

46. An article of manufacture comprising a composition-of-matter, the composition-of-matter comprising a substrate coated, on at least a portion of a surface thereof, by a liposome according to claim 33.

47. A method of treating a synovial joint disorder, the method comprising administering to a subject in need thereof the liposome of claim 33 or a composition comprising the liposome and a carrier.

48. (canceled)

49. The liposome of claim 33 having a therapeutically active agent associated therewith, the liposome or the composition being for delivering the therapeutically active agent to a bodily site of a subject.

50. (canceled)

51. A process of preparing the polymeric compound of claim 1, the process comprising contacting an initiator compound having Formula V: 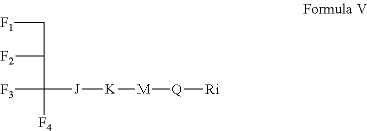
wherein:
F₁, F₂, F₃, F₄, J, K, M and Q are as defined for Formula IV; and
Ri is an electron transfer functional group,
with a plurality of monomers that form said —[Y-L-Z]n-[Y]m- polymeric backbone,
under conditions that promote atom transfer radical polymerization (ATRP).

52-54. (canceled)
